(19) United States
(12) Patent Application Publication
(10) Pub. No.: US 2002/0128208 A1
(43) Pub. Date: Sep. 12, 2002

(54) Nonpeptide agonists and antagonists of vasopressin receptors

(76) Inventors: James P. Snyder, Atlanta, GA (US); Dennis C. Liotta, Atlanta, GA (US); Hariharan Venkatesan, San Diego, CA (US); Minmin Wang, Indianapolis, IN (US); Matthew C. Davis, Ridgecrest, CA (US)

Correspondence Address: KING & SPALDING, 191 PEACHTREE STREET, N.E., ATLANTA, GA, 30303-1763, US

(21) Appl. No.: 10/023,603

(22) Filed: Dec. 17, 2001

Related U.S. Application Data
(60) Provisional application No. 60/255,946, filed on Dec. 15, 2000.

(51) Int. Cl. C07H 17/02; C07F 9/22; A61K 31/7008

(52) U.S. Cl. 514/23; 514/105; 514/227.5; 514/231.2; 514/252.12; 514/319; 514/661; 536/17.2; 544/59; 544/106; 544/157; 544/337; 544/358; 546/21; 546/206; 564/12; 564/453

(57) ABSTRACT

The disclosed invention is a composition agonists and/or antagonists of V₂, V₁ₐ or both receptors, in a host, including animals, and especially humans, using a small molecule or its pharmaceutically acceptable salt or prodrug.

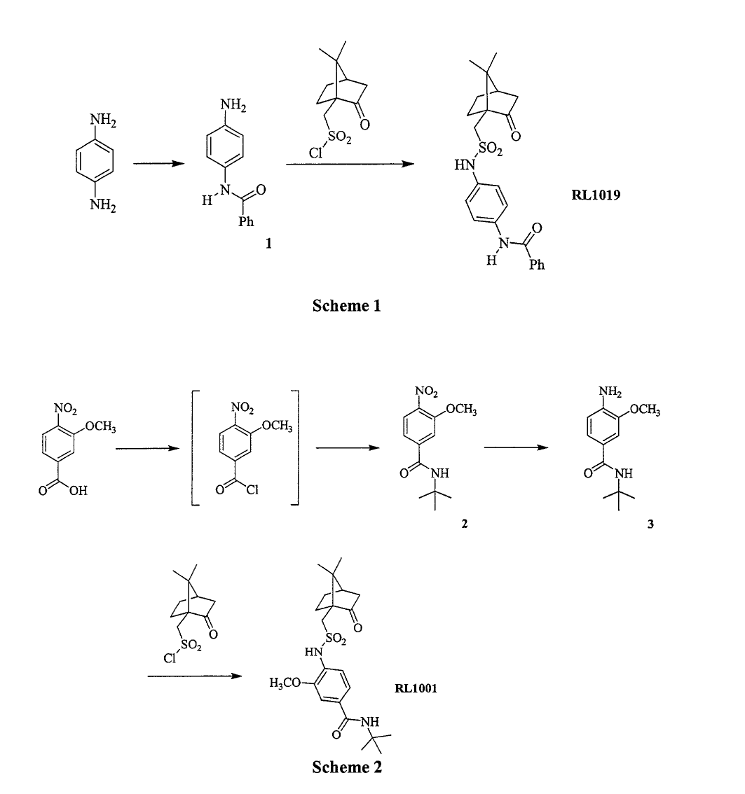

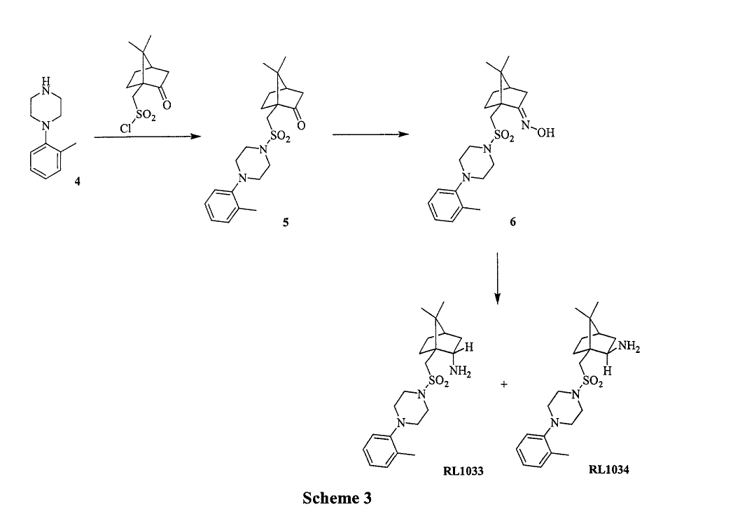

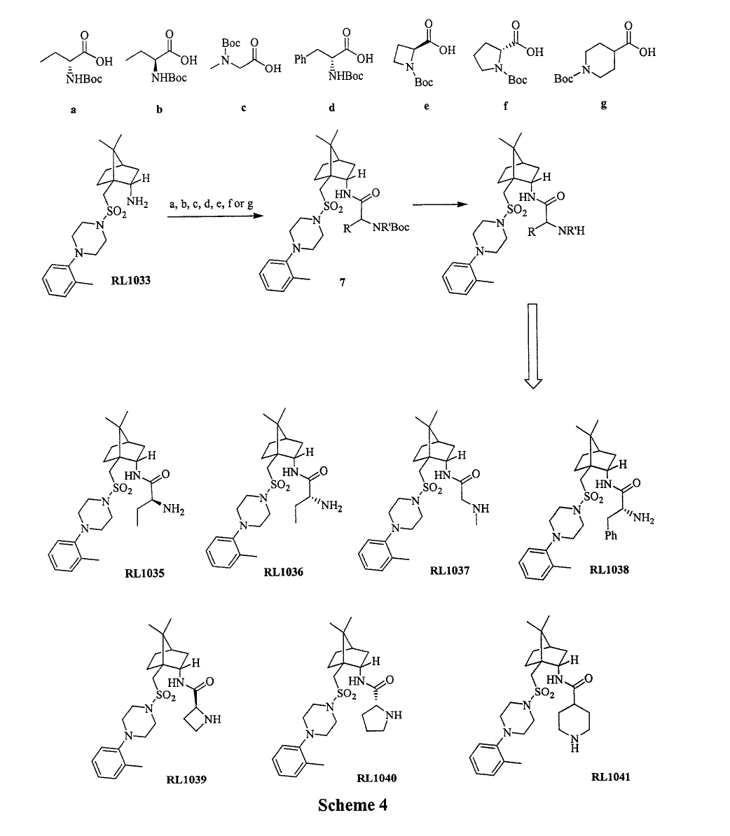

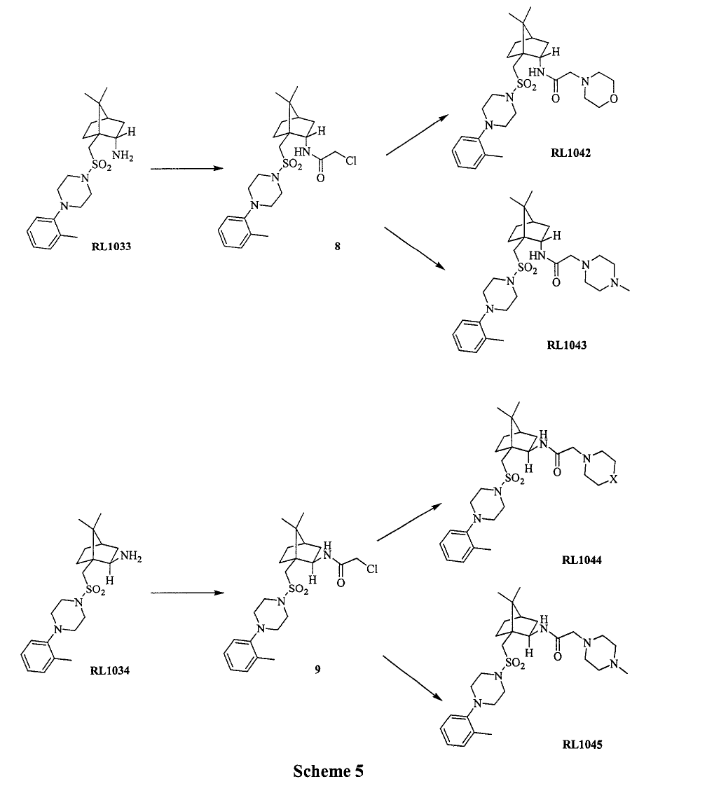

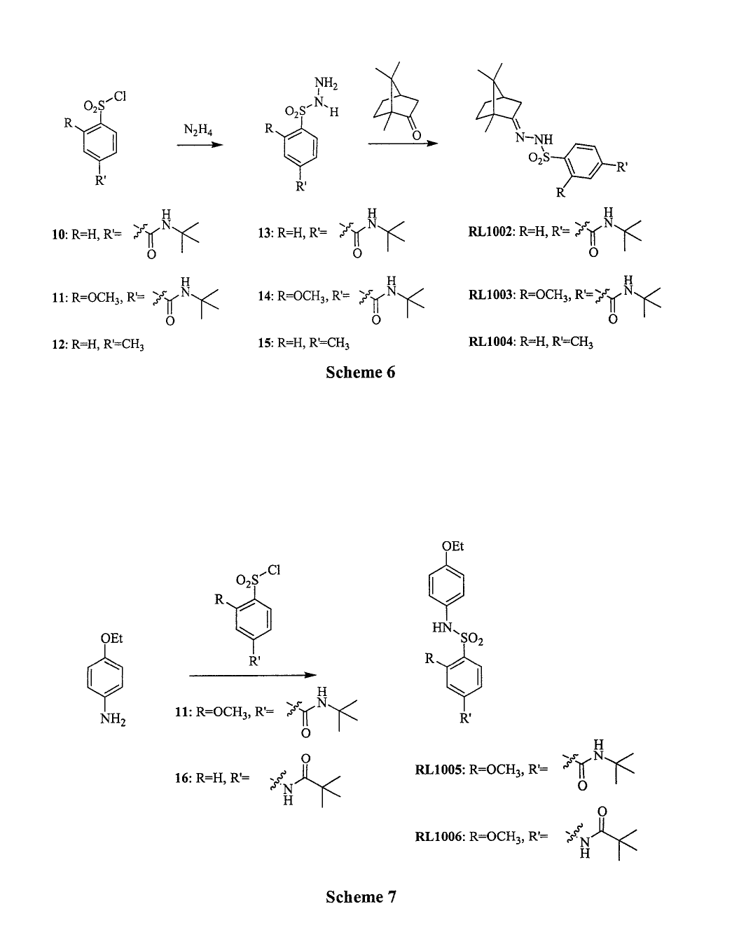

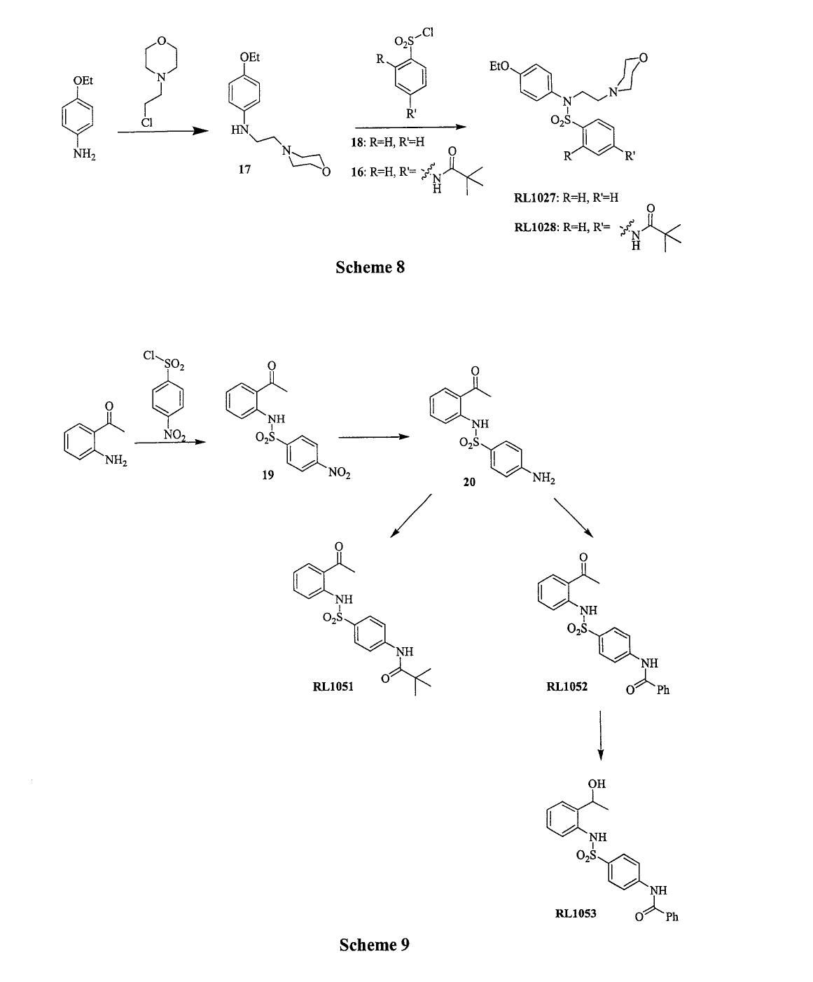

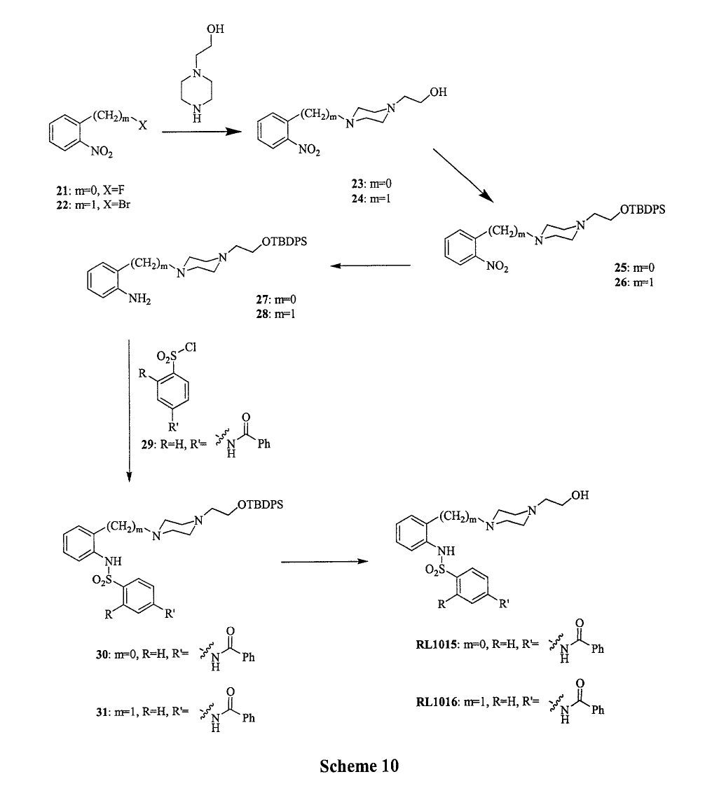

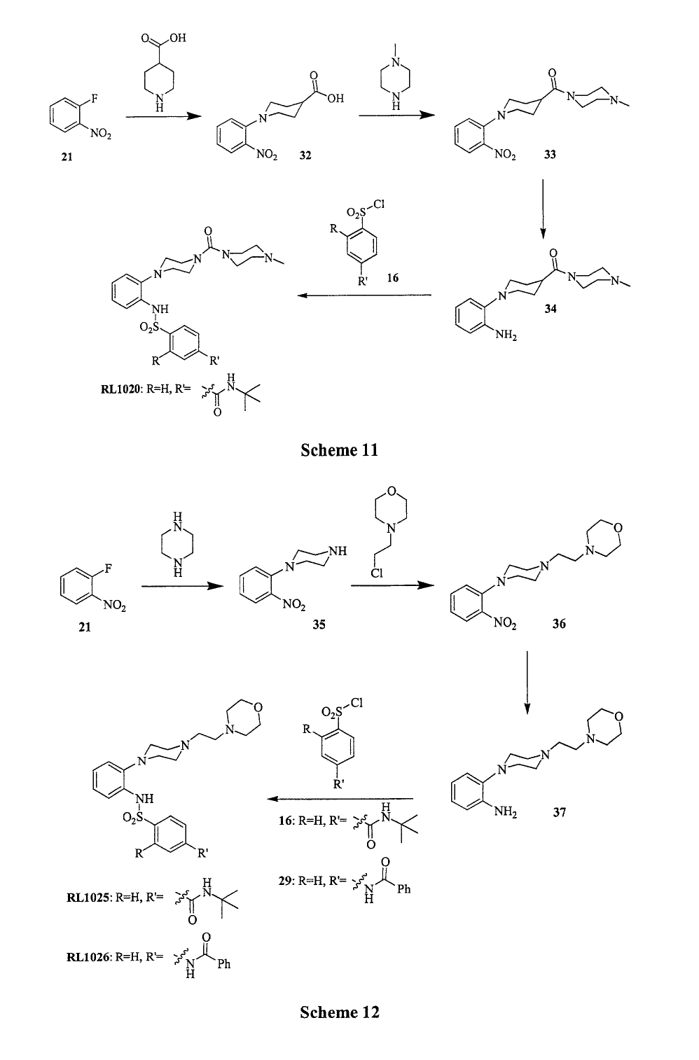

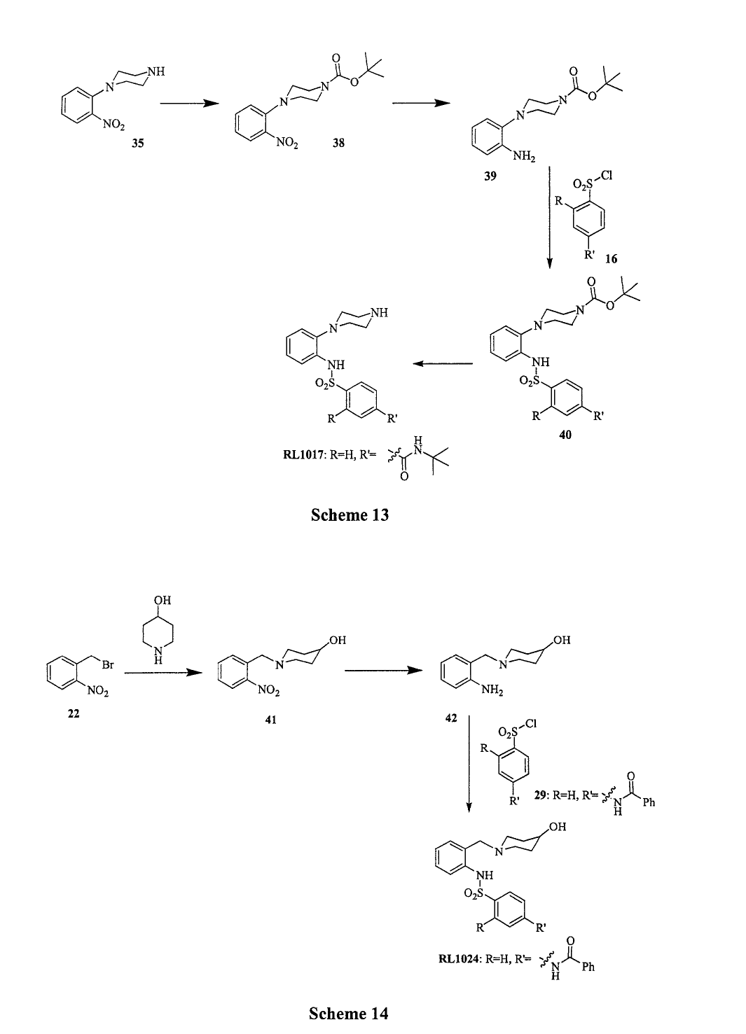

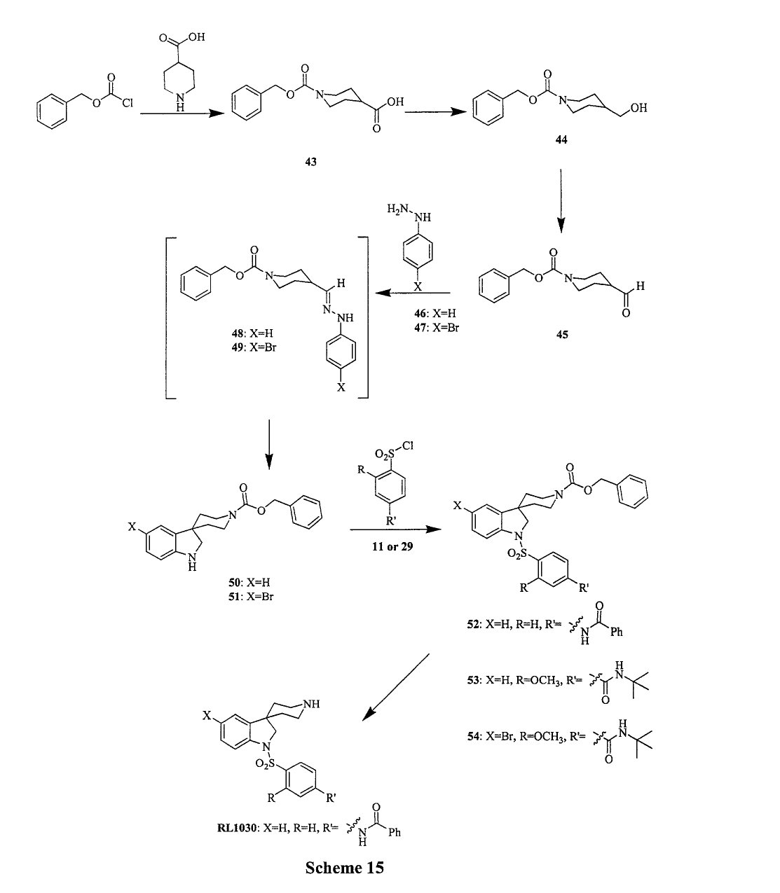

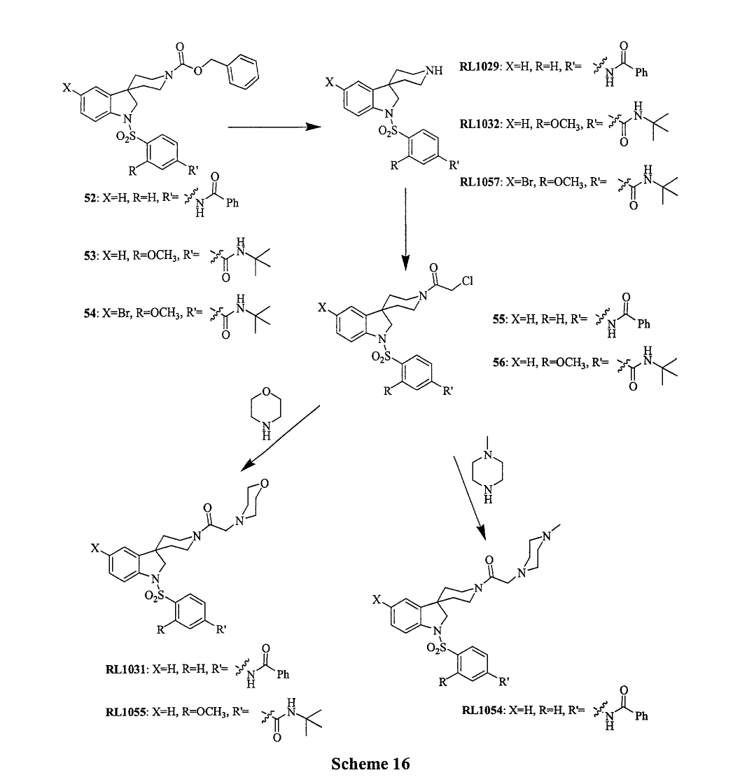

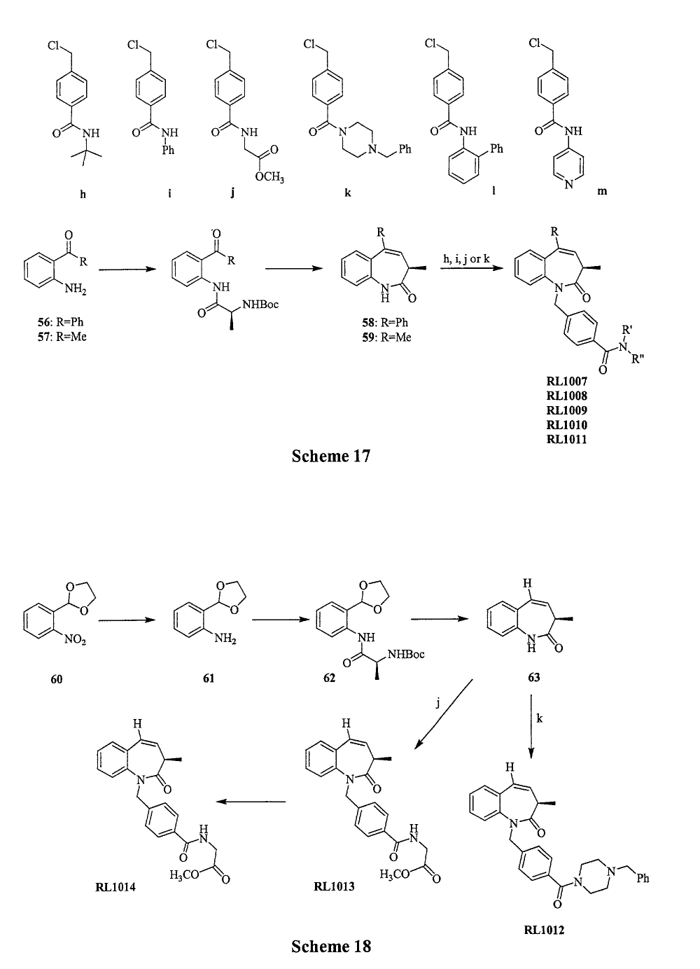

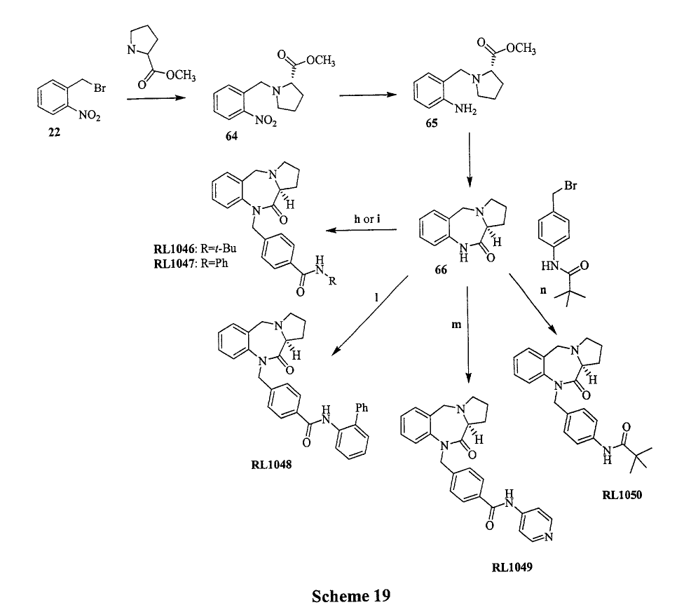

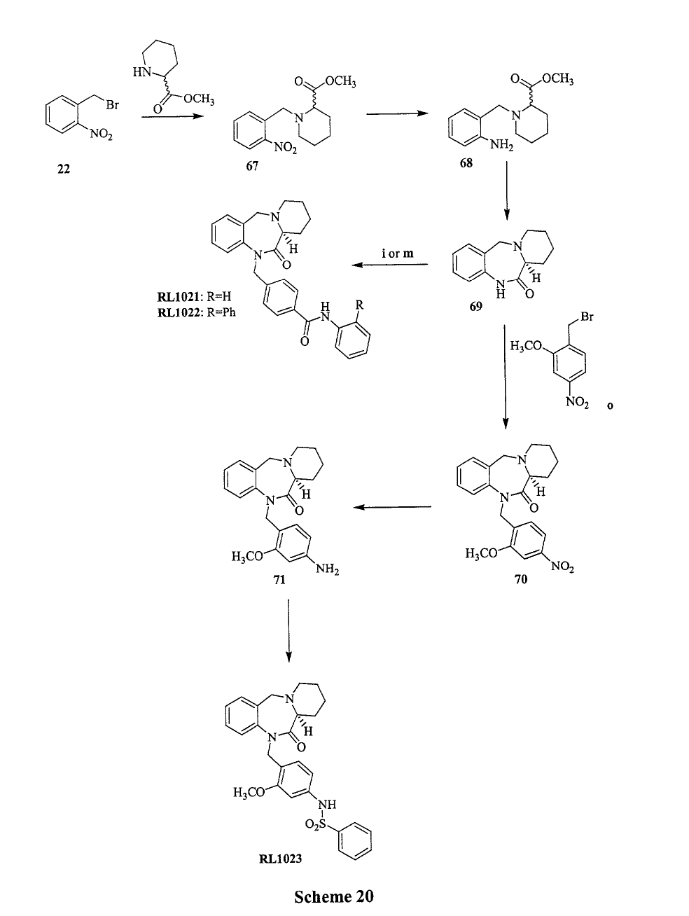

### FIELD OF THE INVENTION

The present invention is in the area of pharmaceutical chemistry and is specifically compounds, pharmaceutical compositions and the uses thereof to selectively block the V₂, V₁ₐ or both receptors. This invention can be used, for example, in the treatment of kidney disorders. This application claims priority to U.S. provisional application 60/255,946 filed on Dec. 15, 2000.

### BACKGROUND OF THE INVENTION

Acute renal failure refers to the abrupt disruption of previously normal kidney function. This serious clinical condition is due to a wide variety of mechanisms including circulatory failure (shock), vascular blockage, glomerulonephritis and obstruction to urine flow. Acute renal failure frequently arises as a complication of abdominal or vascular surgery. Also, due to continued improvements in prenatal care, low birth weight, high-risk neonates may now survive lung and heart problems, only to die from complications of acute renal failure caused by infection or drug toxicity. Of particular clinical importance are cases of acute renal failure associated with trauma, sepsis, postoperative complications or medication, particularly antibiotics (National Center for Health Statistics, 1998, Table III, National Institute of Health, 1990).

Population data from the United States in 1998 further illustrate the nature of the problem. Acute renal failure was cited as a contributing cause in 24,142 deaths. The condition affects people of all ages, but those 65 years and older are almost ten times more likely to be hospitalized for acute renal failure than those ages 45 to 64. Nearly two-thirds of all hospitalizations for acute renal failure occur in people 65 years and older. Of those in that age group, black Americans were nearly twice as likely as Caucasian Americans to be hospitalized for acute renal failure. Acute renal failure is the most costly kidney or urologic condition requiring hospitalization. In 1985 there were 139,134 hospitalizations to for the disease at a cost of $1.3 billion, or $9,329 per hospital discharge. In 1998, the Health Care Financing Administration (HCFA) reported more than 230,000 patients requiring treatments for End Stage Renal Disease (ESRD) or chronic renal failure. The mortality rate of ESRD patients in the U.S. is about 24%. The annual cost of treating ESRD in the U.S. alone in more than $14.5 billion. Therefore, the need for an effective treatment is growing.

In recent years there has been an increase in cases of acute renal failure, which can be attributed in part to medical progress. Most cases today result from the ability to perform complicated surgery in older patients, which can lead to post-operative complications, and the use of complex drugs such as antibiotics that successfully overcome previously fatal diseases. Unfortunately, these drugs can be toxic to the kidneys, particularly in the elderly. Because of the increasing age of the hospital population and advances in complicated medical and surgical techniques, cases of acute renal failure are expected to increase still more in number and significance unless significant advances in treatment modalities are made.

Some advances have been made in understanding the pathophysiology of acute renal failure, including the toxicity of drugs to the kidney and the effects of oxygen deficit and reintroduction. Current treatment of the disorder depends on recognition of the underlying causes. Rapid fluid resuscitation of trauma and burn victims undoubtedly has prevented some cases. Carefully monitored administration of nephrotoxic drugs also has the potential to reduce the incidence of acute renal failure.

Dialysis may be required to prevent death due to accumulating waste products or from fluid overload or chemical imbalance. Currently, over 214,000 patients in the United States receive some form of dialysis. Despite some advances, the mortality rate associated with kidney disease still has not changed in many years. These treatment modalities have focused on preventing further deterioration rather than promoting organ repair. The latter approach would benefit from harnessing cell growth and repair processes.

Diuretics are useful in the treatment of various medical disorders that result in fluid retention, congestive heart failure and hypertension. As such these pharmaceutical compounds can be useful in treating the fluid retention and dilutional hyponatremia associated with a number of severe pathologies such as congestive heart failure, chronic liver disease, hepato-renal syndrome, benign and malignant tumors of the lung, liver and central nervous system. Because diuretics are useful in such a large variety of disorders, their use is widespread but complicated by an associated loss of electrolytes such as potassium that is important to carrying out nervous system functions.

The method of action of some diuretics depend on the control of water absorption which in turn regulates plasma sodium concentrations. Plasma sodium concentrations can be regulated, in part, by absorption and excretion of free water (solute-free water) by the kidney. A greater free water absorption by the kidneys results in a reciprocal decrease in plasma sodium concentrations, while an increase in free water excretion is associated with a rise in sodium concentration. Free water is generated and excreted as a result of the countercurrent multiplication system (CCMS), though the exact mechanism in not known, Sands, J. M.; Kokko, J. P.; Kidney Int., 1996, 50 (suppl 57), S93 argued that this system (for the inner medulla) is a “passive” one, in that though the principle source of energy for operation of the CCMS comes from active outward transport of sodium chloride from the thick ascending limb of Henle, the thin descending and ascending limbs of Henle operate without active transport processes. Rather, these regions are highly dependant on unique epithelial permeability characteristics that would allow sodium chloride to passively diffuse out of the thin limb of Henle into the interstitial medullary space. Furthermore, the inner medullary interstitial fluid must have very high urea concentrations to osmotically balance the high sodium chloride concentration in the lumen of the thin ascending limb. The high medullary urea concentration, in turn, can be regulated through passive diffusion of urea from the papillary collecting duct down its concentration gradient or through active transport via the recently identified urea transporter, UT2. Due to this “passive” regulation, this pathway is unsuitable as a therapeutic target.

However, while the cortical and the medullary collecting duct are impermeable to water in the absence of antidiuretic hormone (ADH), also known as arginine vasopressin (AVP), in the presence of AVP, the water permeability of the collecting duct increases, thereby increasing free water absorption. Furthermore, vasopressin was found to be released from the posterior pituitary gland in response to increased plasma osmolarity detected by brain osmoreceptors or decreased blood volume and blood pressure sensed by low-pressure volume receptors and arterial baroreceptors. Therefore, the hormone mediated mechanism of water reabsorption across the collecting duct cells was examined. Nielsen, S.; et al.; J. Am. Soc. Nephrol., 1999, 10, 647 teaches that AVP specifically binds to a V₂ receptor coupled to a G protein located on the basolateral membrane which initiates a cascade of cyclic AMP dependent signal transduction events (e.g. activation of phospholipase C via GTP activated G protein, Gp, which cleaves the phosphonate bond in phophatidylinositol-4,5-biphosphonate into yield activated inositol-1,4,5-triphosphate and diacyl-glycerol which in turn activate directly and indirectly various other enzymes) that bring about “active” insertion of specific vasopressin regulated water channels, called aquaporin2 (AQP2), into the apical plasma membrane, allowing water permeation from the lumen to the cell and subsequently across the basolateral membrane to the blood. In short, AVP regulates the re-absorption of water, which, in turn, helps control the sodium levels in the blood which determines arterial pressure. (See also, Bichet, D. G.; et al.; Proc. Assoc. Am. Phy., 110 (5), 387.) Vasopressin thus exerts cardiovascular, hepatic, antidiuretic and aggregating effects and effects on the central and peripheral nervous system where vasopressin-induced antidiuresis, mediated by renal epithelial V₂ receptors, helps to maintain normal plasma osmolarity, blood volume and blood pressure as described above.

AVP not only stimulates renal epithelial V₂ receptors, but also both types of vascular V₁ receptors, V₁ₐ and V_{1b}. V₁ antagonists may decrease systemic vascular resistance, increasing cardiac output, inducing increases in total peripheral resistance and altered local blood flow. V₁ antagonists may decrease blood pressure, induced hypotensive effects and thus be therapeutically useful in treatment of some types of hypertension. These receptors also are localized in the liver, coronary, renal, and cerebral vessels, platelets, kidney, uterus, adrenal glands, central nervous system and pituitary gland. Thus, in conditions with vasopressin induced increases in total peripheral resistance and altered local blood flow, V₁ antagonists may be therapeutic agents. V₁ antagonists may decrease blood pressure, induced hypotensive effects and thus be therapeutically useful in treatment of some types of hypertension.

The blockage of V₂, V₁ₐ or both receptors is useful in treating diseases characterized by excess renal reabsorption of free water. Antidiuresis is regulated by the hypothalamic release of vasopressin (antidiuretic hormone) which binds to specific receptors on renal collecting tubule cells. This binding stimulates adenylyl cyclase and promotes the cAMP-mediated incorporation of water pores into the luminal surface of these cells. V₂, V₁ₐ or both receptor antagonists may correct the fluid retention in congestive heart failure, liver cirrhosis, nephritic syndrome, central nervous system injuries, lung disease and hyponatremia.

Agonists of V₂, V₁ₐ or both receptor enhance the action of ADH, increasing the cellular reuptake of water, thereby decreasing sodium concentration. These compounds are potential agents for the treatment of entirely different syndromes, such as diabetes insipius (DI), enuresis, hemophilia, von Willebrand's syndrome, and in the regulation of hemostasis such as an antidote to platelet aggregating agents (Laszlo, F. A. Pharmacol. Rev., 1991, 43, 73; and Drug Investigation, 1990, X (Suppl. 5), 1. Conversely, antagonists of V₂, V₁ₐ or both receptor block the action of ADH, preventing the cellular reuptake of water, thereby increasing sodium concentration. Vasopressin receptor antagonists can affect the regulation of the central and peripheral circulation, especially the coronary, renal and gastric circulation, as well as the regulation of hydration and the release of adrenocorticotrophic hormone (ACTH). These compounds could be useful for treatment of severe hyponatremia, where patients can benefit from both water loss and increased blood sodium. The frequency of hyponatremia is common in the elderly, patients who concomitantly take diuretics and patients in certain disease states such as AIDS, congestive heart failure (where peripheral resistance is increased), cirrhosis with ascites and the syndrome of inappropriate antidiuretic hormone secretion (SIADH).

The vasopressin hormone itself and some of their peptide and non-peptide analogs are used in therapeutics and have been found to be effective against conditions regulated by diuresis and arterial pressure. Several reviews and numerous literature articles may be mentioned: Vasopressin, Gross, P. et al. ed., John Libbey Eurotext, 1993, in particular 243-257 and 549-562; Laszlo, F. A. et al. Clinical perspectives for vasopressin antagonists, Drug News Perspect., 6 (8), (1993); North, W. G. J. Clin. Endocrinol., 73, 1316 (1991); ,Legros, J. J. et at., Prog. Neuro-Pharmacol. Biol. Psychiat., 12, 571, (1998); Andersson, K. E. et al. Drugs Today, 24 (7), 509 (1988); Stump, D. L. et al. Drugs, 39, 38 (1990); Caltabiano, S. et al. Drugs Future, 13, 25 (1988); Mura, Y. et al. Clin. Nephrol., 40, 60 (1993); and Faseb, J., 8 (5), A 587: 3398 (1994).

The following prior art references describe peptide vasopressin antagonists: Manning, M. et al. Chem., 35, 382 (1992); Manning, M. et al. J. Med. Chem., 35, 3895 (1992); Gavras, H. et al. U.S. Pat. No. 5,080,187 (1991); Manning, M. et al. U.S. Pat. No. 5,055,448 (1991); Ali, F. E. U.S. Pat. No. 4,766,108 (1988); Ruffolo, R. R. et al. Drug News and Perspective, 4 (4), 217, (1991) Williams et al. J. Med. Chem., 35, 3905 (1992) which also exhibit weak vasopressin antagonist activity in binding to V₁ and V₂ receptors.

Non-peptide vasopressin antagonist have recently been disclosed, Yamamura, U. et al.Science, 252, 579 (1991); Yammura, Y. et al. Pharmaco. Br. J., 105, 787 (1992); Ogawa et al. EP 514667 A1, EP 038185 A2, WO 91/05549 and U.S. Pat. No. 5,258,510; Yamanouchi Pharm. Co. Ltd. WO 94/04525, WO 94/20473, WO 94/12476 and WO 94/14796; Fujisawa Co. Ltd. EP 620216 A1. Ogawa et al. EP 470514 A disclose carbostyril derivative and pharmaceutical compositions containing the same. Non-peptide oxytocin and vasopressin antagonist have been disclosed by Merck and Co.; Bock, M. G. et al. EP 533242 A and EP 533244A; Erb, J. M. et al. EP 533240 A; Gilbert, K. et al., EP 533243 A.

Peptide vasopressin antagonists suffer from a lack of oral activity and many of these peptides are not selective antagonists since they also exhibit partial agonist activity. In addition, oxytocin is a peptide that is structurally similar to vasopressin.

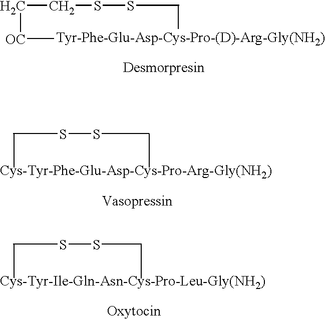

The oxytocin receptors are found on the smooth muscle of the uterus, as well as on myoepithelial cells of the mammary gland, in the central nervous system and in the kidney. The localization of the different receptors is described by Jars, S et al., Vasopressin and oxytocin receptors: an overview, in Progress in Endocrinology; Imura, H.; Shizume, K. Ed., Experta Medica, Amsterdam, 1183 (1988); Presse Medicale, 16 (10), 481 (1987); J. Lab. Clin. Med., 114 (6), 617 (1989); and Pharmacol. Rev., 43 (1), 73 (1991). Vasopressin thus exerts cardiovascular, hepatic, antidiuretic and aggregating effects and effects on the central and peripheral nervous system and in the uterine domain. Oxytocin is involved in parturition, lactation and sexual behavior.

There have been disclosure of some specific V₂, V₁ₐ or both receptor antagonists by various companies.

Sanofi has the following compound SR121463A with a Kᵢ of 1.42 nM.

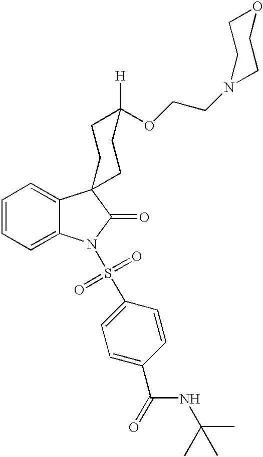

OPC-31260 with a Kᵢ of 21.7nM

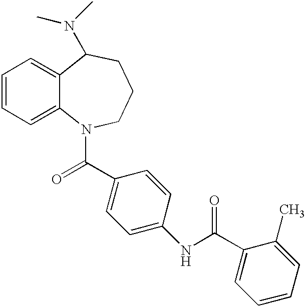

YM35087with a Kᵢ of 1.8nM

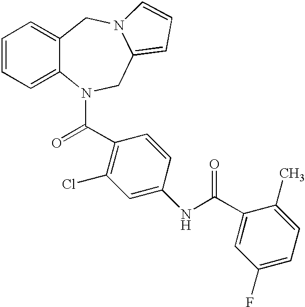

and VPA-985 with a Kᵢ of 0.5 nM

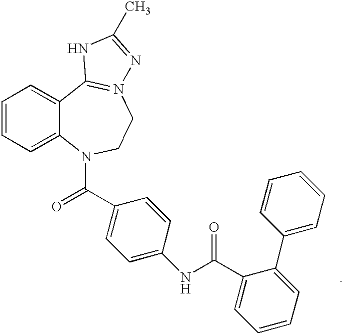

Thus, it is a goal of a desirable goal is to develop an improved diuretic that effectively increases the excretion of urine without depleting the important electrolytes in the treated patient as well as selectively acts on vasopressin over oxytocin.

Another goal is to provide an efficient synthesis to such compounds.

### SUMMARY OF THE INVENTION

Compounds of formula (I) are provided:

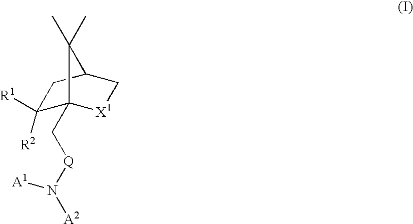

or its pharmaceutically acceptable salt or prodrug thereof, wherein

X¹ is C(═Z¹) or CH₂;

Q is CH₂, C(═Z²), S, S(═Z³), (Z³═)S(═Z⁴), PA³, PA³(═O) or P(═O)₂;

Z¹ and Z² are independently O, S or NA⁴;

Z³ and Z⁴ are independently O or NA⁵ wherein Z³ and Z⁴ both cannot be NA⁵;

A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,

A¹ and A² can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate; (Morpholine)

R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively

R¹ and R² can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

Compounds of formula (II) are provided:

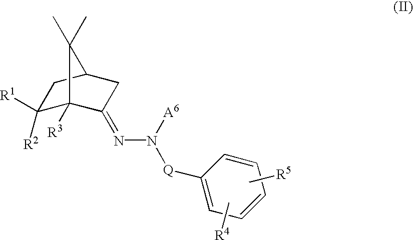

or its pharmaceutically acceptable salt or prodrug thereof, wherein Q, R¹ and R² are defined above;

A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozine, carbamate, thioether anhydride, residue of a natural or synthetic amino acid, or carbohydrate; alternatively

R⁴and R⁵ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

Compounds of formula (III) are provided:

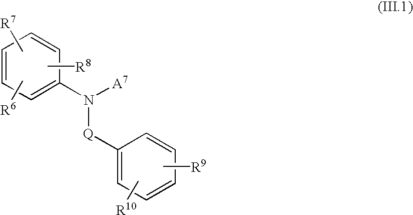

or its pharmaceutically acceptable salt or prodrug thereof, wherein Q is defined above; and

A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively

R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;

wherein in a preferred embodiment, if A⁷ and R^{6/8} independently come together to form a seven-membered bridged compound, then Q cannot be C(═O).

In a specific embodiment, compounds of formula (III) are more explicitly described as

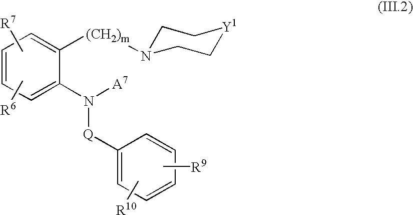

or its pharmaceutically acceptable salt or prodrug thereof, wherein Q, A⁷, R⁶, R⁷, R⁹ and R¹⁰ are defined above;

m is 0 or 1;

Y¹ is O, S, NA⁸ or CR¹¹R¹²; and

A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively

R¹¹ and R¹² can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

In an alternate embodiment, compounds of formula (III) are more explicitly described as

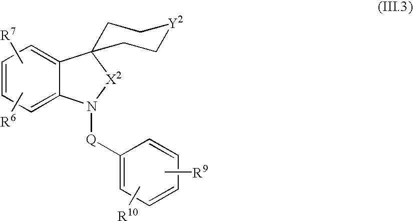

or its pharmaceutically acceptable salt or prodrug thereof, wherein Q, R⁶, R⁷, R⁹ and R¹⁰ are defined above;

Y² is O, S, NA⁹ or CR¹⁵R¹⁶;

X² is C(═Z⁵) or CR¹⁷R¹⁸;

Z⁵ is O, S or NA¹⁰;

A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl; and

R¹⁵, R^{16,} R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

R¹⁵ or R¹⁶ independently cannot be the following moiety:

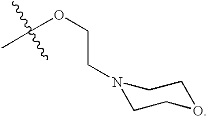

In an alternate embodiment, compounds of formula (III) are more explicitly described as

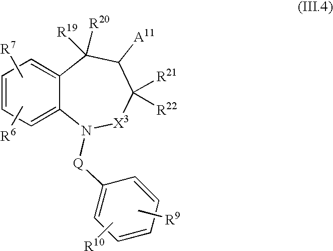

or its pharmaceutically acceptable salt or prodrug thereof, wherein Q, R⁶, R⁷, R⁹ and R¹⁰ are defined above;

X³ is C(═Z⁶);

Z⁶ is O, S or NA¹²;

A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl; and

R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

In an alternate embodiment, R^{19/20} and A¹¹ can come together to form a pi bond.

In another embodiment, CR¹⁹R²⁰ can be C═O.

The present invention also includes the use of a compound of formula I, II or III, or its pharmaceutically acceptable salt or prodrug thereof as V₂ and/or V₁ₐ agonists or antagonists.

The present invention also includes the use of a compound of formula I, II or III, or its pharmaceutically acceptable salt or prodrug thereof in a medical therapy, i.e. as a V₂ and/or V₁ₐ agonists or antagonists, for example as a diuretic.

The present invention also includes the use of a compound of formula I, II or III, or its pharmaceutically acceptable salt or prodrug thereof in the manufacture of a medicament as a V₂ and/or V₁ₐ agonists or antagonists.

The present invention also includes a pharmaceutical composition that include an effective amount of a V₂ and/or V₁ₐ agonists of antagonists of formula I, II or III, or its pharmaceutically acceptable salt or prodrug thereof together with a pharmaceutically acceptable carrier or diluent according to the present invention.

The present invention also includes a pharmaceutical composition that include an effective amount of a V₂ and/or V₁ₐ agonists of antagonists of formula I, II or III, or its pharmaceutically acceptable salt or prodrug thereof in combination or alteration with one or more other V₂ and/or V₁ₐ agonists or antagonists.

The process for preparation of compounds of formula 1, II or III and their pharmaceutically acceptable salts or prodrugs are also disclosed.

### BRIEF DESCRIPTION OF THE FIGURES

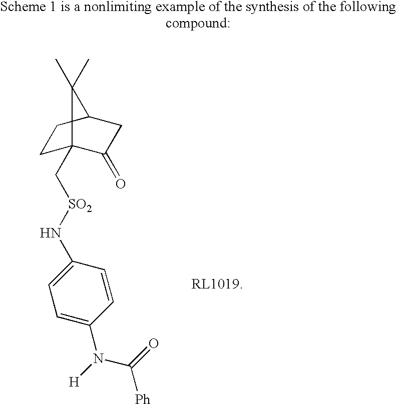

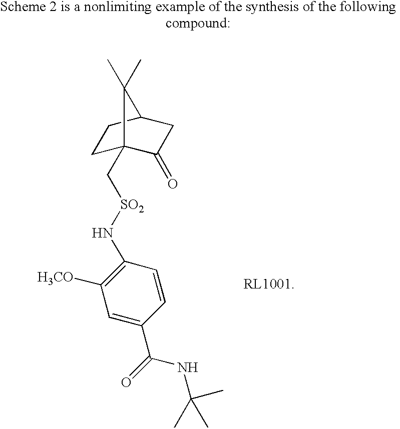

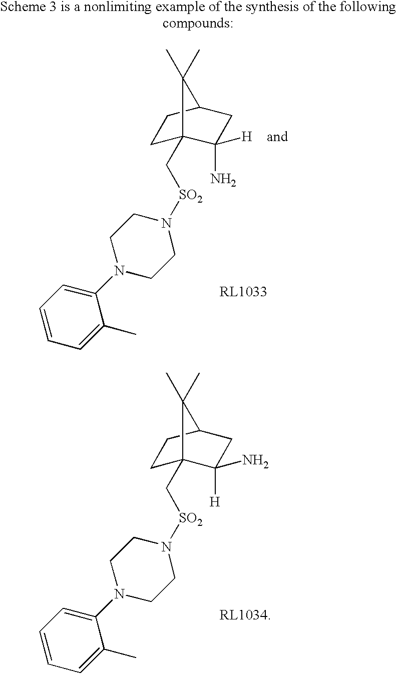

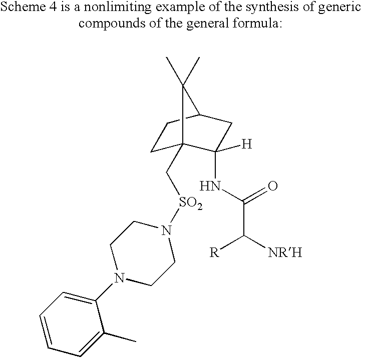

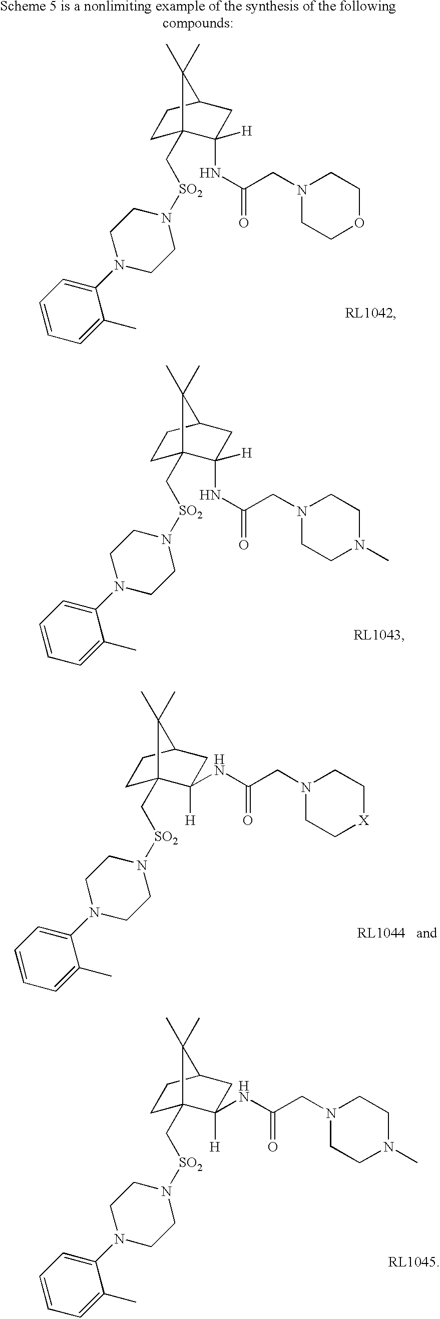

wherein R and R′ independantly can be hydrogen or a lower alkyl; alternatively R and R′ can come together to form a bridged compound comprising a lower alkyl.

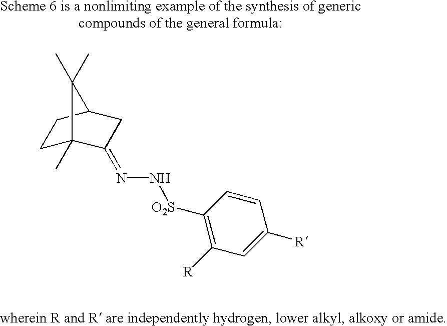

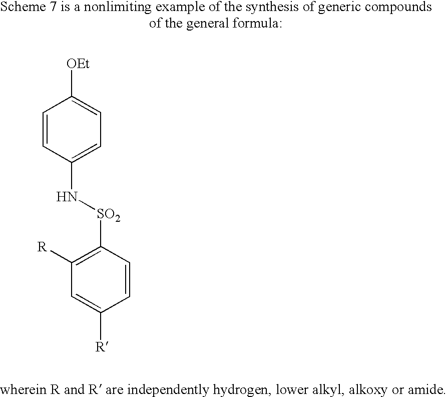

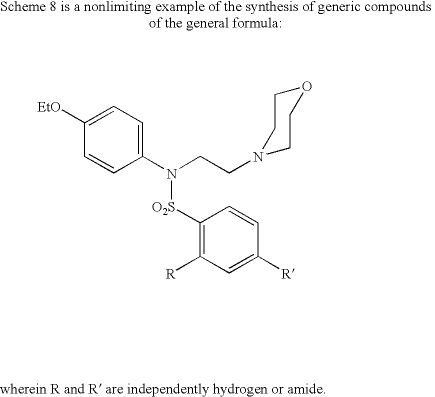

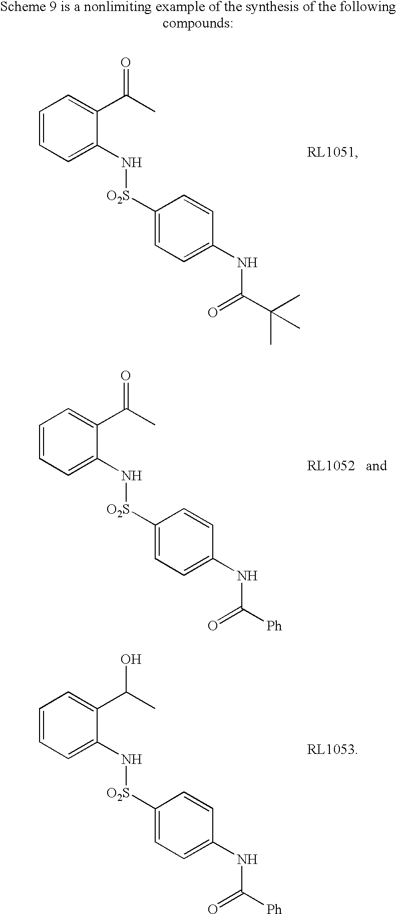

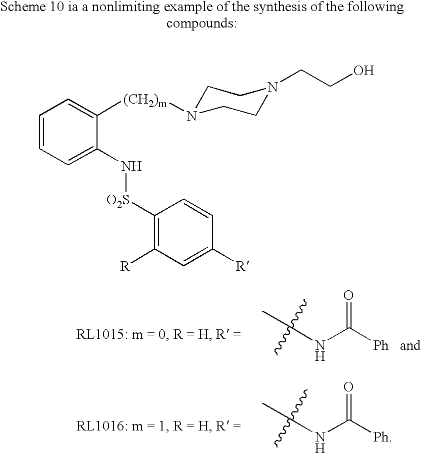

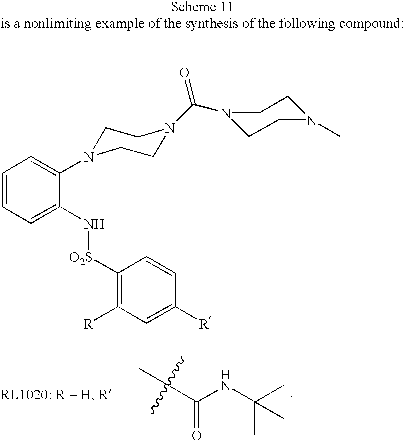

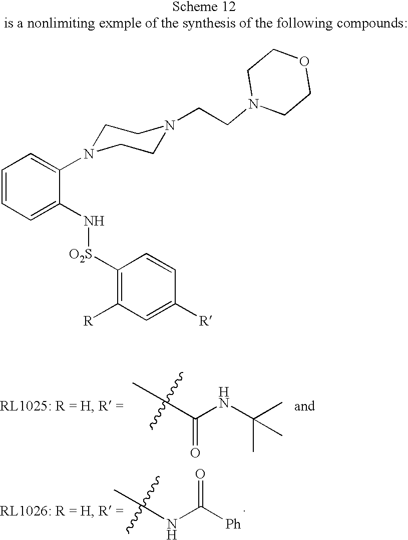

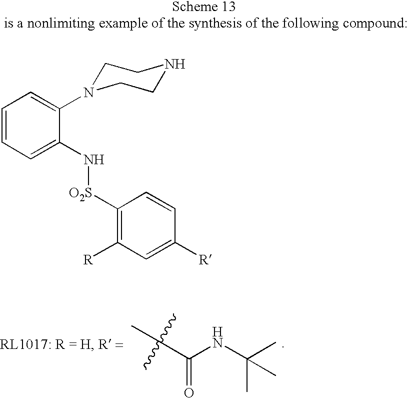

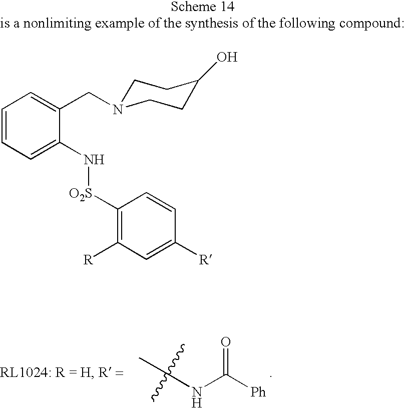

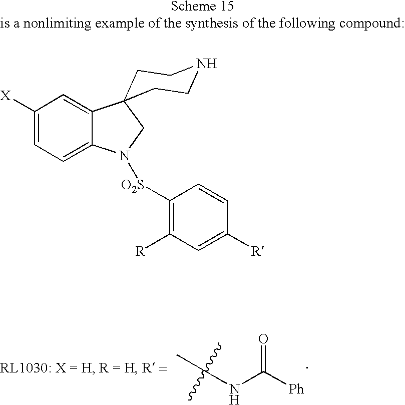

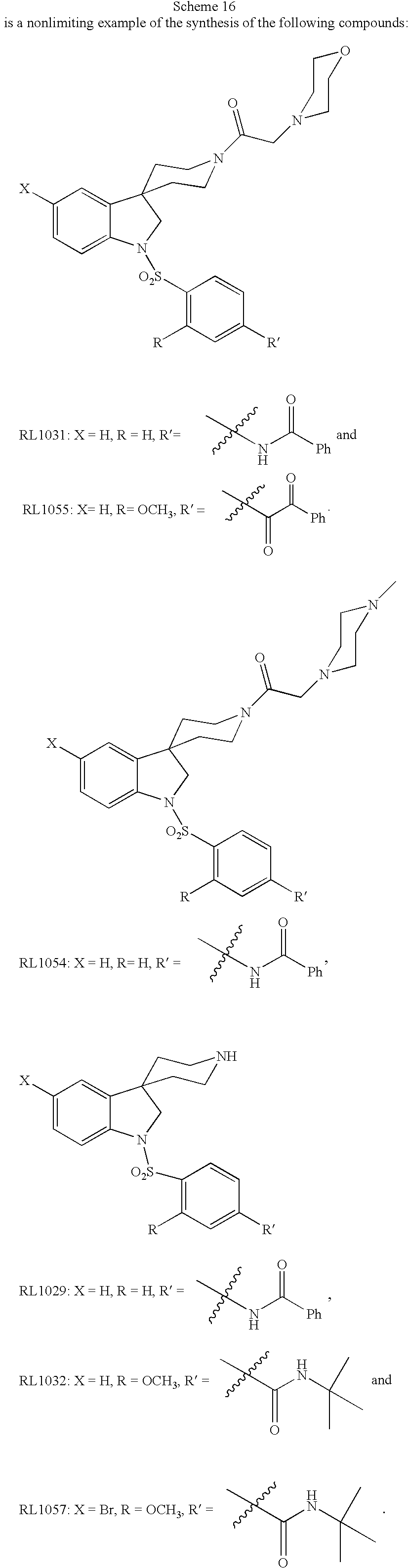

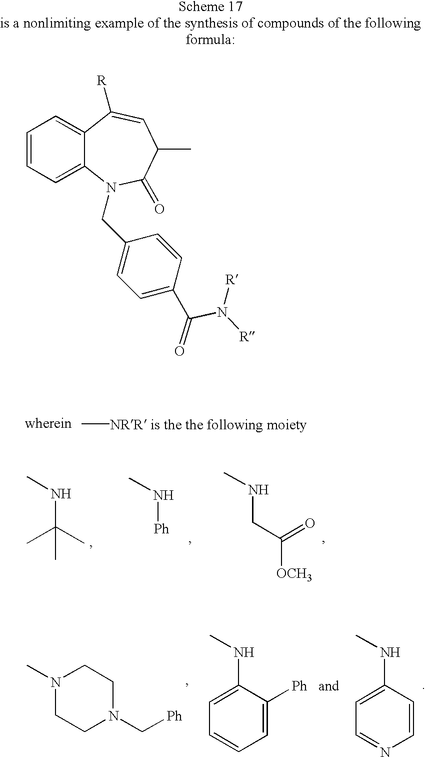

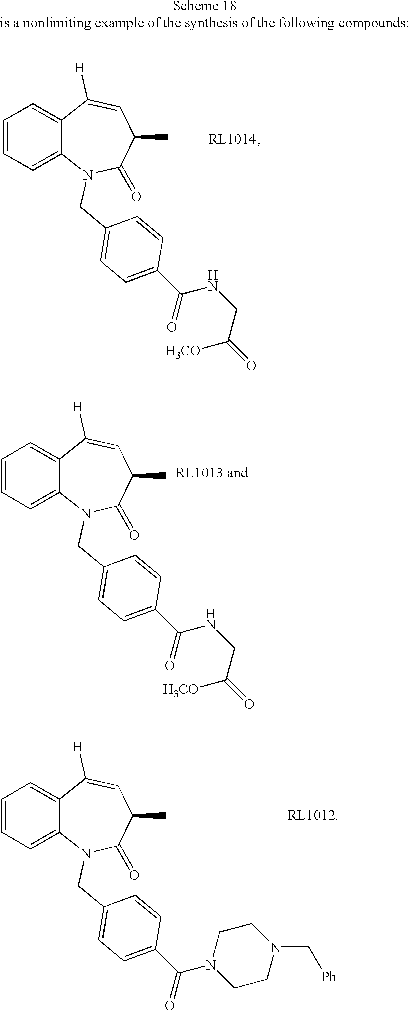

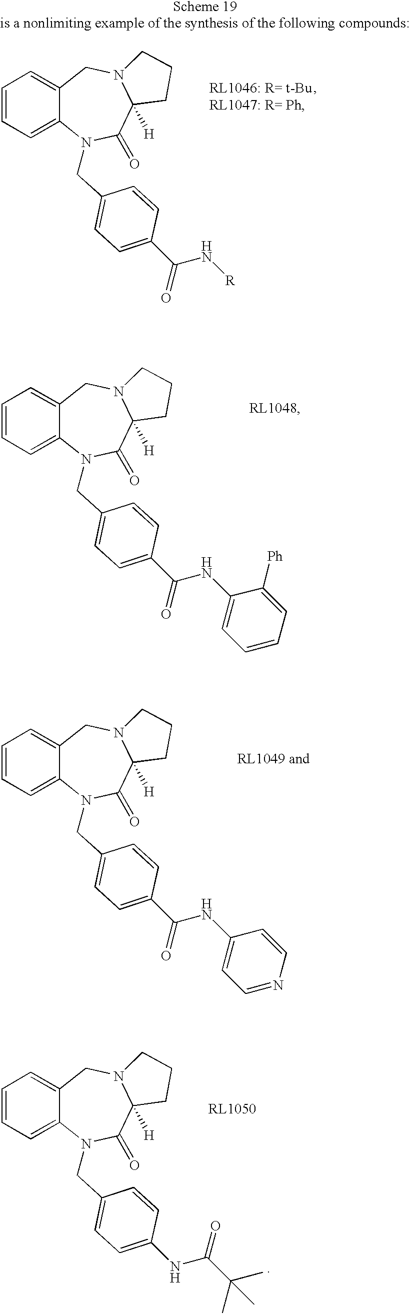

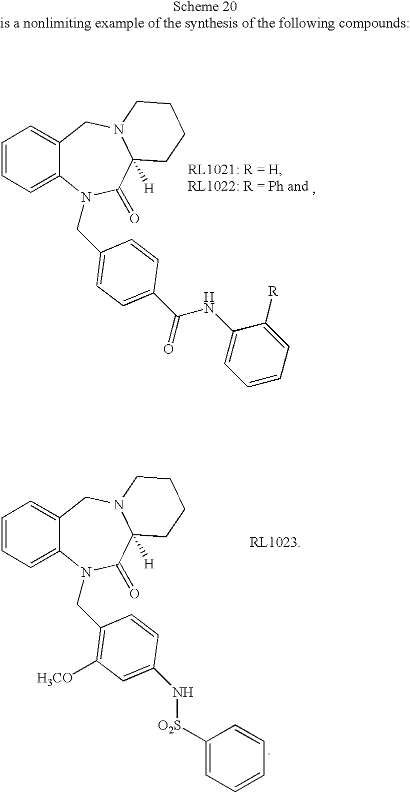

### DETAILED DESCRIPTION OF THE INVENTION

Compounds of formula (I) are provided:

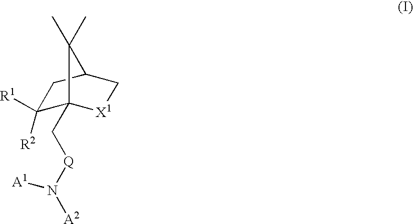

or its pharmaceutically acceptable salt or prodrug thereof, wherein

X¹ is C(═Z¹) or CH₂;

Q is CH₂, C(═Z²), S, S(═Z³), (Z³═)S(═Z⁴), PA³, PA³(═O) or P(═O)₂;

Z¹ and Z² are independently O, S or NA⁴;

Z³ and Z⁴ are independently O or NA⁵ wherein Z³ and Z⁴ both cannot be NA⁵;

A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,

A¹ and A² can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate; (Morpholine)

R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively

R¹ and R² can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

Compounds of formula (II) are provided:

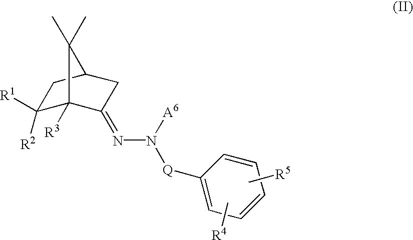

or its pharmaceutically acceptable salt or prodrug thereof, wherein Q, R¹ and R² are defined above;

A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively

R⁴ and R⁵ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

Compounds of formula (III) are provided:

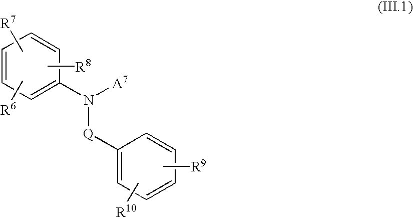

or its pharmaceutically acceptable salt or prodrug thereof, wherein Q is defined above; and

A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively

R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;

wherein in a preferred embodiment, if A⁷ and R^{6/8} independently come together to form a seven-membered bridged compound, then Q cannot be C(═O).

In a specific embodiment, compounds of formula (III) are more explicitly described as

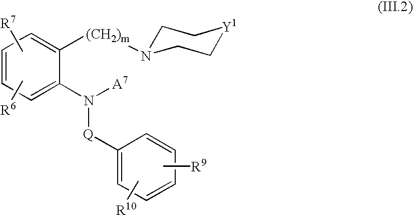

or its pharmaceutically acceptable salt or prodrug thereof, wherein Q, A⁷, R⁶, R⁷, R⁹ and R¹⁰ are defined above;

m is 0 or 1;

Y¹ is O, S, NA⁸ or CR¹¹R²; and

A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;

R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively

R¹¹ and R¹² can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

In an alternate embodiment, compounds of formula (III) are more explicitly described as

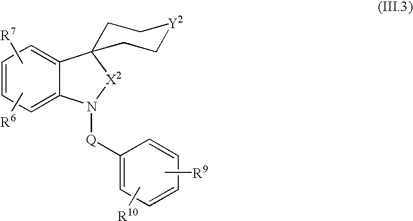

or its pharmaceutically acceptable salt or prodrug thereof, wherein Q, R⁶, R⁷, R⁹ and R¹⁰ are defined above;

Y² is O, S, NA⁹ or CR¹⁵R¹⁶;

X² is C(═Z⁵) or CR¹⁷ R¹⁸;

Z⁵ is O, S or NA¹⁰;

A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl; and

R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbonate, thioether anhydride, residue of a natural or synthetic amino acid, or carbohydrate.

R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

R¹⁵ or R¹⁶ independently cannot be the following moiety:

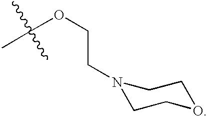

In an alternate embodiment, compounds of formula (III) are more explicitly described as

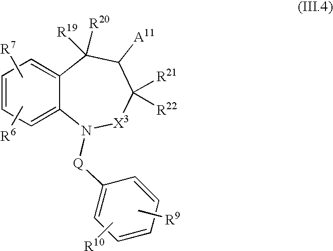

or its pharmaceutically acceptable salt or prodrug thereof, wherein Q, R⁶, R⁷, R⁹ and R¹⁰ are defined above;

Y³ is O, S, or NA¹¹;

X³ is C(═Z⁶);

Z⁶ is O, S or NA¹²;

A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl; and

R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

In an alternate embodiment, R^{19/20} and A¹¹ can come together to form a pi bond.

In another embodiment, CR¹⁹R²⁰ can be C═O.

The present invention also includes the use of a compound of formula I, II or III, or its pharmaceutically acceptable salt or prodrug thereof as V₂ and/or V₁ₐ agonists or antagonists.

The present invention also includes the use of a compound of formula I, II or III, or its pharmaceutically acceptable salt or prodrug thereof in a medical therapy, i.e. as a V₂ and/or V₁ₐ agonists or antagonists, for example as a diuretic.

The present invention also includes the use of a compound of formula I, II or III, or its pharmaceutically acceptable salt or prodrug thereof in the manufacture of a medicament as a V₂ and/or V₁ₐ agonists or antagonists.

The present invention also includes a pharmaceutical composition that include an effective amount of a V₂ and/or V₁ₐ agonists of antagonists of formula I, II or III, or its pharmaceutically acceptable salt or prodrug thereof together with a pharmaceutically acceptable carrier or diluent according to the present invention.

The present invention also includes a pharmaceutical composition that include an effective amount of a V₂ and/or V₁ₐ agonists of antagonists of formula I, II or III, or its pharmaceutically acceptable salt or prodrug thereof in combination or alteration with one or more other V₂ and/or V₁ₐ agonists or antagonists.

The process for preparation of compounds of formula I, II or III and their pharmaceutically acceptable salts or prodrugs are also disclosed.

I. Stereoisomerism and Polymorphism

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

In one embodiment of the invention, the active compound is provided in substantially pure form, i.e. is approximately 95% optically pure or more.

Optically active forms of the compounds can be prepared using any method known in the art, including by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including simulated moving bed chromatography, is used in one embodiment. A wide variety of chiral stationary phases are commercially available.

II. Definitions The term “alkyl,” as used herein, unless otherwise specified, refers to a saturated straight, branched, or cyclic, primary, secondary, or tertiary hydrocarbon, including but not limited to those of C₁ to C₁₆, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms.

The term alkylene or alkenyl refers to a saturated hydrocarbyldiyl radical of straight or branched configuration, including but not limited to those that have from one to ten carbon atoms. Included within the scope of this term are methylene, 1,2-ethanediyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term aryl, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of bromo, chloro, fluoro, iodo, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “aralkyl,” as used herein, and unless otherwise specified, refers to an aryl group as defined above linked to the molecule through an alkyl group as defined above. The term alkaryl or alkylaryl as used herein, and unless otherwise specified, refers to an alkyl group as defined above linked to the molecule through an aryl group as defined above. In each of these groups, the alkyl group can be optionally substituted as describe above and the aryl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxyphenyl; 4-chlorophenyl; 4-methylphenyl; 3,5-ditertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4-tertiarybutylphenyl; 4-tertiarybutylphenylmethyl and the like.

The term “alkylamino” or “arylamino” refers to an amino group that has one or two alkyl or aryl substituents, respectively.

The term “halo” or “halogen,” as used herein, includes bromo, chloro, fluoro and iodo.

The term “heteroatom,” as used herein, refers to oxygen, sulfur, nitrogen and phosphorus.

The term “protected” as used herein and unless otherwise defined refers to a group that is added to a heteroatom to prevent its further reaction or for other purposes. A wide variety of oxygen and nitrogen protecting groups are known to those skilled in the art of organic synthesis.

The term “alkoxy,” as used herein, and unless otherwise specified, refers to a moiety of the structure -O-alkyl, wherein alkyl is as defined above.

The term “acyl,” as used herein, refers to a group of the formula C(O)R′, wherein R′ is an alkyl, aryl, alkaryl or aralkyl group, or substituted alkyl, aryl, aralkyl or alkaryl, wherein these groups are as defined above.

The term “heterocyclic” refers to a nonaromatic cyclic group wherein there is at least one heteroatom, such as oxygen, sulfur, nitrogen, or phosphorus in the ring. The term “heteroaryl” or “heteroaromatic,” as used herein, refers to an aromatic that includes at least one sulfur, oxygen, nitrogen or phosphorus in the aromatic ring. Nonlimiting examples are furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, iso-oxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, 1,2,3-oxadiazole, thiazine, pyridine, pyrazine, pyrimidine or pyridazine, pteridinyl, aziridines, thiophene, furan, pyrrole, isopyrrole, pyrazole, thiazole, isothiazole, 1,2,3-oxadiazole, thiazine, pyridine, pyrazine, piperazine, pyrrolidine, and oxaziranes wherein said heterocyclic or heteroaryl groups can be optionally substituted with one or more substituent selected from halogen, haloalkyl, alkyl, alkoxy, hydroxy, carboxyl derivatives, amido, amino, alkylamino, dialkylamino. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl.

The term amino acid includes naturally occurring and synthetic amino acids, and includes but is not limited to, alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, and histidinyl.

The term “ether” as used herein, refers to oxygen that is disubstitued with independent alkyl groups or two alkyl groups that together formed a ring or a bridge. Some non-limiting examples include 3-(imidazol-1-yl)propoxy, 4-(imidazol-1-yl)butoxy, 5-(imidazol-1-yl)pentoxy, 2-(benzimidazol-1-yl)ethoxy, 3-(benzimidazol-1-yl)-propoxy, 4-(benzimidazol-1-yl)butoxy, 5-(benzimidazol-1-yl)-pentoxy, 2-(tetrahydrobenzimidazol-1-yl)ethoxy, 3-(tetrahydrobenzimidazol-1-yl)propoxy, 4-(tetrahydrobenzimidazol-1-yl)butoxy, 5-(tetrahydrobenzimidazol-1-yl)pentoxy, ethoxy, n-propoxy, or isopropoxy. The ethers also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “sulfoxy,” as used herein, refers to a pentavalent sulfur moiety. Non-limiting examples include methanesulphonyloxy, ethanesulphonyloxy, n-propane-sulphonyloxy, isopropanesulphonyloxy, n-butanesulphonyloxy, benzenesulphonyloxy, 4-fluorobenzenesulphonyloxy, 4-bromobenzenesulphonyloxy, 4-methylbenzenesulphonyloxy, 4-methoxybenzene-sulphonyloxy, 3,4-dichlorobenzenesulphonyloxy, phenylmethanesulphonyloxy, 2-phenylethanesulphonyloxy, or 3-phenylpropanesulphonyloxy. The sulfoxy group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “amide” as used herein, refers to a carbonyl moiety wherein the non-alkyl moiety is formed from an amine. Some non-limiting examples are formylamino, acetylamino, propionylamino, butanoylamino, isobutanoylamino, pentanoylamino, 3-methyl-butanoylamino, hexanoylamino, methoxycarbonylamino, ethoxycarbonylamino, n-propoxycarbonylamino, isopropoxycarbonylamino, benzamido, cyclopentylcarbonylamido, cyclohexylcarbonylamido, cycloheptylcarbonyl-amido, phenylacetylamido, cyclohexylacetylamido, cyclohexylpropionylamido, N-methyl-formylamino, N-methylacetylamino, N-ethyl-acetylamino, N-ethyl-propionylamino, N-ethyl-butanoylamino, N-ethyl-pentanoylamino, N-ethyl-3-methyl-butanoylamino, N-ethyl-cyclohexylcarbonylamido, N-ethyl-cycloheptylcarbonylamido, N-ethyl-phenylacetylamido, N-ethyl-3-phenyl-propionylamido, N-ethyl-cyclopentylacetylamido, N-ethyl-3-cyclopentylpropionylamido, N-ethyl-cyclohexylacetylamido, N-ethyl-3-cyclohexylpropionylamido, N-ethyl-cycloheptylacetyl-amido, N-ethyl-3-cycloheptylpropionylamido, N-n-propylformylamino, N-n-propyl-acetylamino, N-isopropyl-formylamino, N-isopropylacetylamino, N-isopropyl-propionylamino, N-isopropyl-butanoylamino, N-isopropyl-(3-methyl-butanoyl)amino, N-isobutyl-formylamino, N-isobutyl-acetylamino, N-isobutylpropionylamino, N-isobutyl-butanoylamino, N-isobutyl-isobutanoylamino, N-isobutylpentanoylamino, cyclohexylacetylamino, N-isopropyl-cyclohexylcarbonylamino, N-isopropyl-cyclohexylacetylamino, N-isopropyl-3-(cyclohexyl)-propionylamino, N-n-butyl-cyclohexylcarbonylamino, N-n-butyl-cyclohexylacetylamino, 3,3-tetramethylene-glutaramino, 3,3-pentamethylene-glutaramino, 2,2-dimethyl-glutaramino, 3-methylglutaramino, 3-ethyl-glutaramino, 3-ethyl-3-methyl-glutaramino, 3-methyl-maleic acid amido, or morpholinocarbonylamino. The amide group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “imide,” as used herein, refers to a carbonyl derivative wherein the carbonyl carbon is double bonded to a nitrogen rather than a oxygen. Some non-limiting examples include 2-phenyl-maleic acid imido, 7-Fluoro-6-(3,4,5,6-tetrahydrophthalimido)-4-(2-propynyl)-1,4-benzoxazin-3(2H)-one, Phthalimide Potassium Salt, N-(Hydroxymethyl)phthalimide, N-(trichloro-methylmercapto)-D4-tetrahydrophthalimide, N-(Trichloromethylthio)phthalimide, tetrahydrophthalimide, cis-, Diethyl N-hydroxynaphthalimide phosphate, N-Butylphthalimide, 3,4,5,6-Tetrachlorophthalimide, 3,6-Diaminophthalimide, 4-Amino-1,8-naphthalimide, N-(Chloromethyl)phthalimide, 5-methoxy-phthalimino, 4,5-dimethoxy-1-oxo-isoindolin-2-yl, 2-carboxyphenylmethylamino, 2-carboxyphenylmethylenecarbonylamino, pyrrolidino, 2-methylpyrrolidino, 3-ethylpyrrolidino, 3-isopropylpyrrolidino, piperidino, 3-methylpiperidino, 4-methylpiperidino, 4-ethylpiperidino, 4-isopropylpiperidino, hexamethyleneimino, 3-methylhexamethyleneimino, 4-methylhexamethyleneimino, 3-ethylhexamethyleneimino, 4-isopropylhexamethyleneimino, 3,3-dimethyl-pyrrolidino, 3,4-dimethyl-pyrrolidino, 3,3-dimethyl-piperidino, 2-oxo-hexamethyleneimino, propanesultam-1-yl, butanesultam-1-yl, pentanesultam-1-yl, 3,3-tetramethylene-glutarimino, 3,3-pentamethyleneglutarimino, 2,2-dimethyl-glutarimino, 3-methyl-glutarimino, 3,3-dimethyl-glutarimino, 3-ethyl-glutarimino, 3-ethyl-3-methyl-glutarimino, 1,3-cyclopentanedicarbonylimino, 2,4-dimethyl-glutarimino, or 2,4-di-n-propyl-glutarimino. The imide also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “sulfamoyl” is a hexavalent sulfur covalently bound to at least two oxygens and a nitrogen. Some non-limiting examples include methanesulphonylamino, ethanesulphonylamino, n-propanesulphonylamino, isopropanesulphonylamino, n-butanesulphonylamino, n-pentanesulphonylamino, n-hexanesulphonylamino, benzenesulphonylamido, 4-fluorobenzenesulphonamido, 4-chlorobenzenesulphonamido, 4-bromobenzenesulphonamido, 4-methylbenzenesulphonamido, 4-methoxybenzenesulphonamido, phenylmethanesulphonyl-amido, 2-phenylethanesulphonylamido, N-methylethanesulphonylamino, N-methyl-n-propanesulphonylamino, N-methylisopropanesulphonylamino, N-methyl-benzenesulphonylamido, N-methyl-4-fluorobenzene-sulphonamido, N-methyl-4-chlorobenzenesulphonamido, N-methyl-4-bromobenzenesulphonamido, N-methyl-4-methylbenzenesulphonamido, N-methyl-4-methoxybenzenesulphonamido, N-methyl-phenylmethanesulphonylamido, N-methyl-2-phenylethanesulphonylamido, N-methyl-3-phenylpropanesulphonylamido, N-methylnaphthalen-1-yl-sulphonamido, N-methyl-naphthalen-2-yl-sulphonylamido, N-ethylmethanesulphonylamino, N-ethyl-ethanesulphonylamino, N-ethyl-n-propanesulphonylamino, N-ethyl-isopropanesulphonylamino, N-ethyl-n-butanesulphonylamino, N-ethyl-n-pentanesulphonylamino, N-ethyl-naphthalen-1-yl-sulphonamido, or N-ethyl-naphthalen-2-yl-sulphonylamido. The sulfamoyl group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “carbamide” refers to a carbonyl flanked on both sides by a nitrogen. Some non-limiting examples include aminocarbonylamino, methylaminocarbonylamino, dimethylaminocarbonylamino, N-methylaminocarbonyl-methylamino, N-(dimethylaminocarbonyl)methylamino, N-dimethylaminocarbonyl-ethylamino, N-dimethylaminocarbonyl-isopropylamino, N-(dimethylaminocarbonyl)-n-pentylamino, N-methylaminocarbonylethylamino, N-methylaminocarbonyl- n-pentylamino, N- methylamino-carbonyl-n-hexylamino, N-((n-hexyl)-methylaminocarbonyl)-amino, cyclohexylamino-carbonylamino, N-cyclohex ylaminocarbonyl-methylamino, N-cyclohexylaminocarbonyl-ethylamino, N-cyclohexylamino-carbonyl-n-butylamino, N-cyclohexylaminocarbonyl-isobutylamino, N-cyclohexylaminocarbonyl-n-pentylamino, N-cyclohexylaminocarbonyl-n-hexylamino, N-cyclohexylaminocarbonyl cyclohexylamino, N-(ethylcyclohexylaminocarbonyl)-methylamino, N-(propyl-cyclohexylaminocarbonyl)-methylamino, N-(n-butyl-cyclohexylaminocarbonyl)-methylamino, allylaminocarbonylamino, benzylaminocarbonylamino, N-benzyl aminocarbonyl-isobutylamino, or phenylaminocarbonyl-amino. The carbamide group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “thio” refers to a sulfur covalently bound to a hydrogen or a carbon based group. Some non-limiting examples include methylmercapto, ethylmercapto, n-propylmercapto, isopropylmercapto or n-butylmercapto, ethylthio, n-propylthio or isopropylthio group. The thio group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “ester” refers to a carbonyl flanked by an alkoxy group and a carbon based group. Some non-limiting examples include hydroxycarbonyl, methoxycarbonyl, ethoxycarbonyl, n-propyloxycarbonyl, isopropyloxycarbonyl, n-butyloxycarbonyl, isobutyloxycarbonyl, tert.butyloxycarbonyl, n-pentyloxycarbonyl, isoamyloxycarbonyl, n-hexyloxy-carbonyl, cyclopentyloxycarbonyl, cyclohexyloxycarbonyl, benzyloxycarbonyl, 1-phenylethyloxycarbonyl, 2-phenylethyloxycarbonyl, 3-phenylpropyloxycarbonyl, methoxymethoxycarbonyl, cinnamyloxycarbonyl, acetoxymethoxycarbonyl, propionyloxymethoxycarbonyl, 1-(3-phenylpropyloxycarbonyloxy)-ethoxycarbonyl, or 1-(cinnamyloxycarbonyloxy)-ethoxycarbonyl. The ester group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, pbosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “urethane” or “carbamate” refers to —OC(O)NR⁴R⁵ in which R⁴ and R⁵ are independently selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the carbamide optimally comprise a phenyl group. The term “lower carbamide” refers to a carbamide group in which the non-carbonyl moiety is a lower alkyl. The carbamide group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

As used herein, the term “substantially free of” or “substantially in the absence of” refers to a composition that includes at least 95% to 98% by weight, and even more preferably 99% to 100% by weight, of the designated enantiomer of that nucleoside. In a preferred embodiment, in the methods and compounds of this invention, the compounds are substantially free of enantiomers. Similarly, the term “isolated” refers to a compound composition that includes at least 95% to 98% by weight, and even more preferably 99% to 100% by weight, of the compound, the remainder comprising other chemical species or enantiomers.

The term “enantiomerically enriched” is used throughout the specification to describe a compound which includes at least about 95%, preferably at least 96%, more preferably at least 97%, even more preferably, at least 98%, and even more preferably at least about 99% or more of a single enantiomer of that compound. When a nucleoside of a particular configuration (D or L) is referred to in this specification, it is presumed that the nucleoside is an enantiomerically enriched nucleoside, unless otherwise stated.

The term “host,” as used herein, refers to a multicellular organism in which the disorders mediated by vasopressin can occur, including animals, and preferably a human. Alternatively, the host is cell with a vasopressin receptor, whose function can be altered by the compounds of the present invention. The term host specifically refers to any cell line that mimics a vasopression mediated disorder, either from natural or unnatural causes (for example, from genetic mutation or genetic engineering, respectively), and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

III. Pharmaceutically Acceptable Salts and Prodrugs

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The term “pharmaceutically acceptable prodrug” is used throughout the specification to describe any pharmaceutically acceptable form (such as an ester, phosphate ester or salt of an ester or a related group) of a disclosed compound which, upon administration to a patient, provides the active parent compound. Pharmaceutically acceptable prodrugs refer to a compound that is metabolized, for example hydrolyzed or oxidized, in the host to form the compound of the present invention. Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the compound. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. The compounds of this invention either possess V₂, V^{1a} or both receptor agonistic and/or antagonistic activity, or are metabolized to a compound that exhibits such activity.

IV. Pharmaceutical Compositions

Pharmaceutical compositions based upon a compound of formula I, II or III can be prepared that include the above-described compound or its salt or prodrug in a therapeutically effective amount for the treatment of any of the indications described herein, including as an agonist and/or antagonist of V₂, V₁ₐ or both receptors, optionally in combination with a pharmaceutically acceptable additive, carrier or excipient. The therapeutically effective amount may vary with the condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in orally administrable form, but formulations may be administered via parenteral, intravenous, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification, etc.).

In certain pharmaceutical dosage forms, the prodrug form of the compound, especially including acylated (acetylated or other) and ether derivatives, phosphate esters and various salt forms of the present compounds, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound as an agonist and/or antagonist of V₂, V₁ₐ or both receptors.

The amount of compound included within therapeutically active formulations, according to the present invention, is an effective amount of an agonist and/or antagonist of V₂, V₁ₐ or both receptors. In general, a therapeutically effective amount of the present compound in pharmaceutical dosage form usually ranges from about 0.1 mg/kg to about 100 mg/kg or more, depending upon the compound used, the condition or infection treated and the route of administration. For purposes of the present invention, a prophylactically or preventively effective amount of the compositions, according to the present invention, falls within the same concentration range as set forth above for therapeutically effective amount and is usually the same as a therapeutically effective amount.

Administration of the active compound may range from continuous (intravenous drip) to several oral administrations per day (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those which aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

In particularly preferred embodiments according to the present invention, the compounds and compositions are used as agonists and/or antagonists of V₂, V₁ₐ or both receptors. Preferably, to treat, prevent or delay the onset of a V₂, V₁ₐ or both receptors related dysfunction, the compositions will be administered in oral dosage form in amounts ranging from about 250 micrograms up to about 1 gram or more at least once a day, preferably, or up to four times a day. The present compounds are preferably administered orally, but may be administered parenterally, topically or in suppository form.

The compounds according to the present invention, because of their low toxicity to host cells in certain instances, may be advantageously employed prophylactically an agonist and/or antagonist of V₂, V₁ₐ or both receptors or to prevent or promote the occurrence of clinical symptoms associated with V₂, V₁ₐ or both receptor activity. Thus, the present invention also encompasses methods for the prophylactic treatment of V₂, V₁ₐ or both receptor related symptoms. In this aspect, according to the present invention, the present compositions are used as agonists and/or antagonists of V₂, V₁ₐ or both receptors. This prophylactic method comprises administration to a patient in need of such treatment, or who is at risk for the development of V₂, V₁ₐ or both receptor associated disease, an amount of a compound according to the present invention effective for alleviating, preventing or delaying the onset of the symptoms. In the prophylactic treatment according to the present invention, it is preferred that the agonist or antagonist utilized should be low in toxicity and preferably non-toxic to the patient. It is particularly preferred in this aspect of the present invention that the compound which is used should be maximally effective at the V₂, V₁ₐ or both receptor and should exhibit a minimum of toxicity to the patient. In the case of V₂, V₁ₐ or both receptors inhibition or activation, compounds according to the present invention, which may be used to treat these disease states, may be administered within the same dosage range for therapeutic treatment (i.e., about 250 micrograms up to 1 gram or more from one to four times per day for an oral dosage form) as a prophylactic agent to prevent the inhibition or activation of the V₂, V₁ₐ or both receptor, or alternatively, to prolong the onset of a V₂, V₁ₐ or both receptorsassociated disease, which manifests itself in clinical symptoms.

In addition, compounds according to the present invention can be administered in combination or alternation with one or more antidiuretic, V₂, V₁ₐ or both receptors inhibitor or V₂, V₁ₐ or both receptors activator, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

This invention is further illustrated in the following sections. The working examples contained therein are set forth to aid in an understanding of the invention. This section is not intended to, and should not be interpreted to, limit in any way the invention set forth in the claims that follow thereafter.

V. Detailed Description of Process Steps

A. Process for Manufacturing Compounds of the General Structure I

1. Preparation of Starting Material, Method 1

The starting material for this process is an appropriately protected primary or secondary amine, which can be purchased or can be prepared by any known means including standard coupling and protection techniques. In one embodiment, the particular amine can be morpholine, N-methylpiperazine or any other cyclic amine, which can be purchased. In another embodiment, the particular amine is prepared from p-phenylenediamine according to the following protocol.

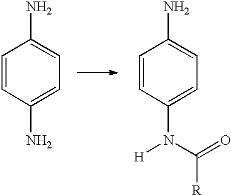

The primary amine is condensed with a carboxylic acid in a compatible solvent at a suitable temperature with the appropriate coupling reagent to yield the corresponding amide. Possible coupling reagents are any reagents that promote coupling, including but not limiting to, carbodiimides such as 1,3-dicyclohexylcarbodiimide (DCC) and 1-(3-dimethylaminopropyl)-3-ethylcarbodiimide hydrochloride (EDC), or Mitsunobu type reagents such as diisopropyl azodicarboxylate and diethyl azodicarboxylate (DEAD) with triphenylphosphine.

The condensation reaction can be carried out at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably THF.

2. Preparation of Starting Material, Method 2

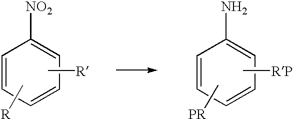

Alternatively, the aniline can be derived from a nitrobenzene derivative. The functional groups off the aromatic ring first may be protected with any known protecting group including silyl and acyl protecting groups. The nitro group can then be reduced using any reducing agents, such as pressurized hydrogen gas over palladium.

The protection and reduction reactions can be carried out at any temperature that achieves the desired result, i.e., that is suitable for the reaction to proceed at an acceptable rate without promoting decomposition or excessive side products. The preferred temperature for these reactions is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably THF. Protic solvent for the reduction, such as methanol, ethanol and water, though preferably methanol.

3. Preparation of Camphor Derivative

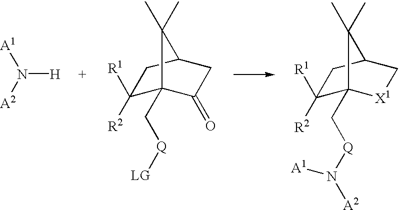

The coupling of the primary or secondary amine with a camphor derivative can be achieved using any suitable base followed by an aqueous work up. For example the coupling can be promoted by diisopropylethylamine and extracted from water.

This reaction can be accomplished at any temperature that achieves the desired result, i.e., that is suitable for the reaction to proceed at an acceptable rate without promoting decomposition or excessive side products. The preferred temperature is again room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably THF.

In another embodiment, the ketone moiety of the camphor ring can be further derivatized by any known means including standard reduction and substitution techniques. For example, the ketone can be reacted with hydoxylamine hydrochloride to form the oxime. Additionally, the oxime can be reduced using a standard reducing agent such as pressurized hydrogen gas, preferably 60 psi, over Raney Nickel with a proton source, usually an alcohol such as 2-methoxyethanol. These primary amines can then be further substituted to form various secondary and tertiary amines using a suitable base followed by an aqueous work up; or, the primary amines can be further derivatized to form amides when condensed with carboxylic acids with the aid of standard coupling reagent. Possible coupling reagents are any reagents that promote coupling, including but not limiting to, carbodiimides such as 1,3-dicyclohexylcarbodiimide (DCC) and 1-(3-dimethylaminopropyl)-3-ethylcarbodiimide hydrochloride (EDC), or Mitsunobu type reagents such as diisopropyl azodicarboxylate and diethyl azodicarboxylate (DEAD) with triphenylphosphine. Alternatively, the amide can be formed by coupling the amine with the appropriate acid chloride in the presence of a mild base, such as triethylamine.

These reactions can be accomplished at any temperature that achieves the desired result, i.e., that is suitable for the reaction to proceed at an acceptable rate without promoting decomposition or excessive side products. The preferred temperature for the substitution of the ketone to an oxime is 70° C. The preferred temperature for the reduction of the oxime is room temperature. The preferred temperature for substitution of the primary amine to form a secondary or tertiary amine is again room temperature. The preferred temperature for the derivatization of the primary amine into an amide is from 0° C. to room temperature.

Any reaction solvent can be selected that can achieve the necessary temperature, can solubilize the reaction components and inert to the reagents, with the exception of the reduction step. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof. The preferred solvent for the substitution of the ketone to a oxime is pyridine. The preferred solvent for substitution of the primary amine to form a secondary or tertiary amine is THF. The preferred solvent for the derivatization of the primary amine into an amide is also THF. For the reduction step, the solvent is preferably protic; some non-limiting examples are alcohol such as methanol, ethanol or 2-methoxyethanol, though preferably 2-methoxyethanol.

B. Process for Manufacturing Compounds of the General Structure II

1. Preparation of Hydrazine Starting Material

The starting material for this process is an appropriately protected hydrazine which can be purchased or can be prepared by any known means including standard coupling and protection techniques. In one embodiment, the particular hydrazide is a sulfonhydrazide, such as p-toluenesulfonhydrazide, which can be prepared using the following protocol.

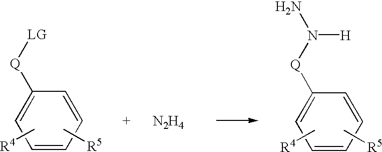

The aryl moiety can be functionalized by hydrazine in the presence of a mild base such as triethylamine.

This reaction can be accomplished at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably toluene.

2. Preparation of Camphor Derivative

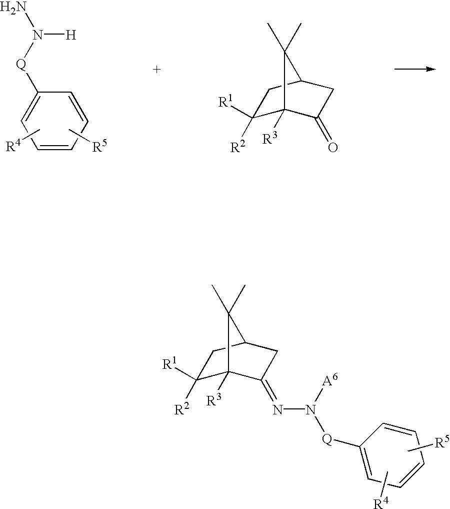

The condensation of the hydrazine with a camphor derivative can be achieved with elevated temperatures. For example the condensation can be achieved with refluxing toluene using a Dean-Stark trap to remove the generated water.

This reaction can be accomplished at any temperature that achieves the desired result, i.e., that is suitable for the reaction to proceed at an acceptable rate without promoting decomposition or excessive side products. The preferred temperature is 110° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably toluene.

C.1 Process for Manufacturing Compounds of the General Structure III, First Embodiment

1. Preparation of Aniline Derivative

The starting material for this process is an appropriately protected aniline and a suitably derivatized chloride, preferably a derivative of benzenesulfonylchloride, which can be purchased or can be prepared by any known means including standard coupling and protection techniques. In one embodiment, the particular aniline is 2-aminoacetophenone and the chloride is 4-nitrobenzenesulfonylchloride, which can be purchased. The coupling of the aniline and the chloride can be prepared using the following protocol.

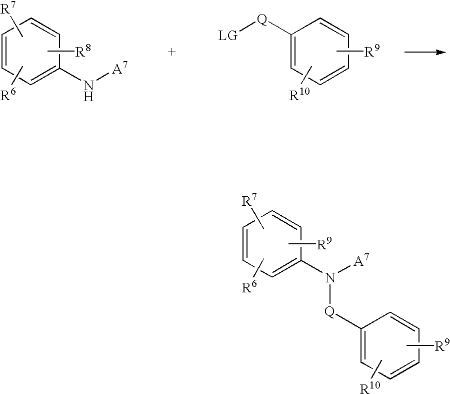

The coupling of the aniline with a chloride derivative can be achieved with a mildly basic solvent such as pyridine.

This reaction can be accomplished at any temperature that achieves the desired result, i.e., that is suitable for the reaction to proceed at an acceptable rate without promoting decomposition or excessive side products. The preferred temperature is 0° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably pyridine.

2. Preparation of Further Derivatives

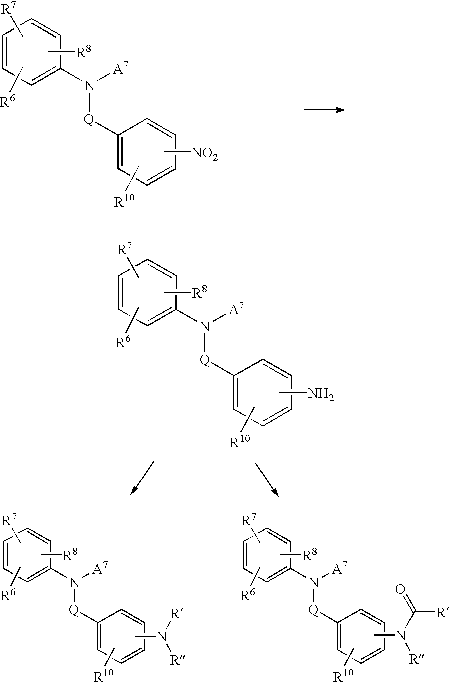

In a particular embodiment, the nitro moiety of the benzene ring can be further derivatized by any known means including standard reduction and substitution techniques. For example, the nitro can be reduced using a standard reducing agent such as pressurized hydrogen gas, preferably 40 psi, over Raney Nickel with a proton source, usually an alcohol such as ethanol. These primary amines can then be further substituted to form various secondary and tertiary amines using a suitable base followed by an aqueous work up; or, the primary amines can be further derivatized to form amides when condensed with carboxylic acids with the aid of standard coupling reagent. Possible coupling reagents are any reagents that promote coupling, including but not limiting to, carbodiimides such as 1,3-dicyclohexylcarbodiimide (DCC) and 1-(3-dimethylaminopropyl)-3-ethylcarbodiimide hydrochloride (EDC), or Mitsunobu type reagents such as diisopropyl azodicarboxylate and diethyl azodicarboxylate (DEAD) with triphenylphosphine. Alternatively, the amide can be formed by coupling the amine with the appropriate acid chloride in the presence of a mild base, such as triethylamine.

These reactions can be accomplished at any temperature that achieves the desired result, i.e., that is suitable for the reaction to proceed at an acceptable rate without promoting decomposition or excessive side products. The preferred temperature for substitution of the primary amine to form a secondary or tertiary amine is again room temperature. The preferred temperature for the derivatization of the primary amine into an amide is from 0° C. to room temperature.

Any reaction solvent can be selected that can achieve the necessary temperature, can solubilize the reaction components and inert to the reagents, with the exception of the reduction step. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof. The preferred solvent for substitution of the primary amine to form a secondary or tertiary amine is THF. The preferred solvent for the derivatization of the primary amine into an amide is also THF. For the reduction step, the solvent is preferably a mixture of a protic solvent and THF; some non-limiting examples of protic solvents are alcohols such as methanol, ethanol or 2-methoxyethanol.

C.2 Process for Manufacturing Compounds of the General Structure III, Second Embodiment

1. Preparation of Aniline Starting Material

The key starting material for this process is an appropriately substituted aniline;

the aniline can be purchased or can be prepared by any known means including standard coupling and reduction techniques. In one embodiment, the particular aniline is prepared from a selected phenyl halide or benzyl halide, for example by formation of the appropriately substitued nitro benzene followed by reduction of the nitro group according to the following protocol.

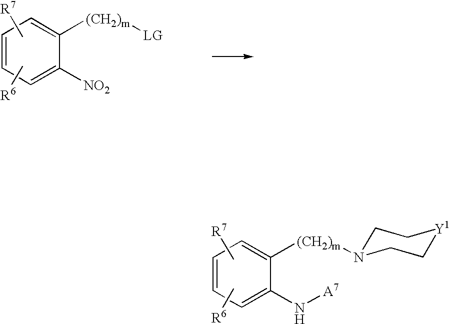

The coupling of the aryl moiety with the appropriate heterocycle can be achieved with elevated temperatures or a mild base, such as potassium carbonate. After optional protection of the functional groups on Y¹ with a standard protecting group such as silyl and carbonyl groups, the substituted nitro benzene then can be reduced using standard reducing agents, such as pressurized hydrogen gas, preferably 30 psi, over Raney Nickel in conjunction with a protic solvent, usually an alcohol such as ethanol.

These reactions can be accomplished at any temperature that achieves the desired result, i.e., that is suitable for the reaction to proceed at an acceptable rate without promoting decomposition or excessive side products. The preferred temperature for the coupling is 80° C. or, if a base is used, room temperature . The preferred temperature for the reduction is room temperature.

Any reaction solvent can be selected that can achieve the necessary temperature, can solubilize the reaction components and inert to the reagents. Non-limiting examples for the coupling reaction are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof. Non-limiting examples for the reduction are any protic solvent including, but not limited to methanol, ethanol and 2-methoxyethanol.

These primary amines can then be further substituted to form various secondary amines using a suitable base followed by an aqueous work up.

These reactions can be accomplished at any temperature that achieves the desired result, i.e., that is suitable for the reaction to proceed at an acceptable rate without promoting decomposition or excessive side products. The preferred temperature for substitution of the primary amine to form a secondary amine is room temperature.

Any reaction solvent can be selected that can achieve the necessary temperature, can solubilize the reaction components and inert to the reagents, with the exception of the reduction step. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, preferably THF.

2. Preparation of Further Derivatives

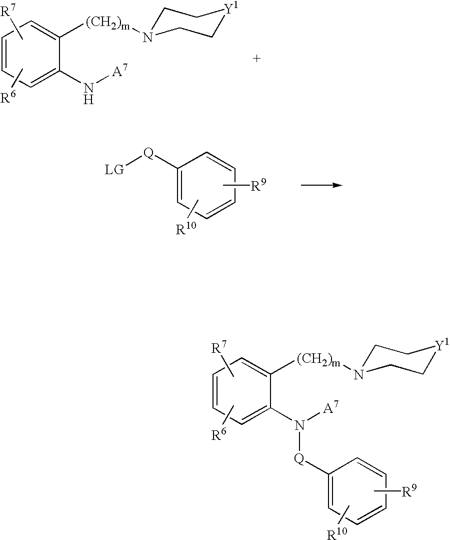

The coupling of the primary or secondary amine with an aryl derivative can be achieved with a mild base. For example the coupling can be promoted by triethylamine and extracted from water.

This reaction can be accomplished at any temperature that achieves the desired result, i.e., that is suitable for the reaction to proceed at an acceptable rate without promoting decomposition or excessive side products. The preferred temperature is 0° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably dichloromethane.

Lastly, the optional protection of the functional groups on Y¹ with silyl or carbonyl protecting groups can be deprotected using standard methods.

C.3 Process for Manufacturing Compounds of the General Structure III, Third Embodiment

1. Preparation of Aldehyde Starting Material

The key starting material for this process is an appropriately substituted aldehyde; the aldehyde can be purchased or can be prepared by any known means including standard protection, reduction and oxidation techniques. In one embodiment, the particular aldehyde is prepared from a selected carboxylic acid, for example by protecting the functional groups on Y², then reducing the substituted carboxylic acid followed by oxidation of the primary alcohol according to the following protocol.

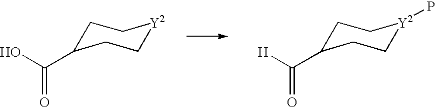

The carboxylic acid may be protected at the Y² position by any means known in the art, including with silyl and carbonyl groups. Then, the carboxylic acid can be reduced with standard reducing agents, such as borane followed by a hydroxide, such as sodium hydroxide, to form the primary alcohol. Finally, the alcohol can be oxidized with a standard oxidizing agent, such as pyridinium chlorochromate (PCC) to give the appropriately functionalized and protected aldehyde.

These reactions can be accomplished at any temperature that achieves the desired result, i.e., that is suitable for the reaction to proceed at an acceptable rate without promoting decomposition or excessive side products. The preferred temperature for the protection is 10° C. to room temperature. The preferred temperature for the reduction is 0° C. The preferred temperature for the oxidation is room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof. The preferred solvent for the reduction is THF. The preferred solvent for the oxidation is dichloromethane. The preferred solvent on the other hand is any polar solvent including, but not limited to methanol, ethanol and water, though preferably water.

2. Preparation of Subsequent Hydrazine

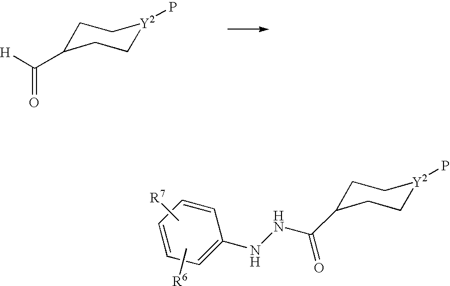

The aldehyde can be coupled with an aryl hydrazine to give the desired derivitized hydrazine without the aid of a base, though one may be used if desired.

This reaction can be accomplished at any temperature that achieves the desired result, i.e., that is suitable for the reaction to proceed at an acceptable rate without promoting decomposition or excessive side products. The preferred temperature for the coupling is 0° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably dichloromethane.

3. Preparation of Spiroindoline Derivative

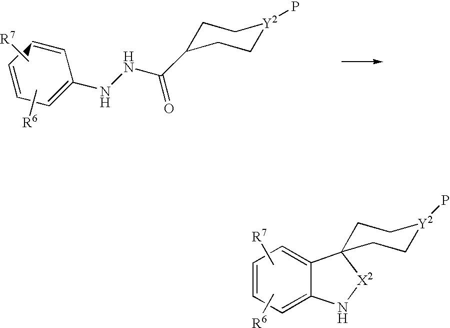

The hydrazine can be cyclized with the aid of an acid, such trifluoroacetic (TFA), to give the desired spiroindoline, preferably in the same pot as the previous reaction, i.e. the formation of the hyrazine from the aldehyde.

This reaction can be accomplished at any temperature that achieves the desired result, i.e., that is suitable for the reaction to proceed at an acceptable rate without promoting decomposition or excessive side products. The preferred temperature for the cyclization is room temperature to 0° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably dichloromethane.

4. Preparation of Spiroindoline Derivative

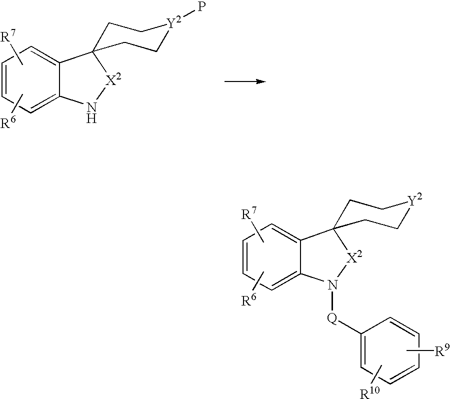

The coupling of the primary or secondary amine with an aryl derivative can be achieved with a mild base. For example the coupling can be promoted by pyridine and extracted from water.

This reaction can be accomplished at any temperature that achieves the desired result, i.e., that is suitable for the reaction to proceed at an acceptable rate without promoting decomposition or excessive side products. The preferred temperature is 0° C.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably dichloromethane.

Lastly, the optional protection of the functional groups on Y¹ with silyl or carbonyl protecting groups can be deprotected using standard methods.

C.4 Process for Manufacturing Compounds of the General Structure III, Fourth Embodiment

1. Preparation of Aldehyde Starting Material

The key starting material for this process is an appropriately substituted amine; the amine can be purchased or can be prepared by any known means including standard reduction and coupling techniques. In one embodiment, the particular amine is prepared from a selected aniline, for example by coupling the aniline according to the following protocol.

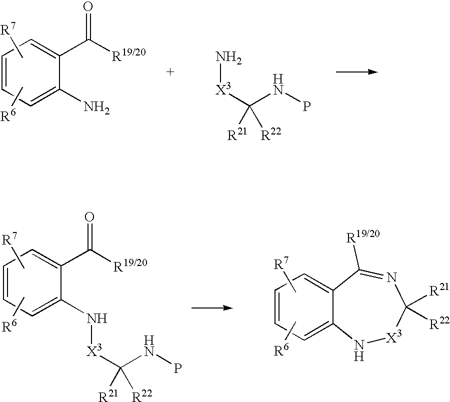

The aniline can be coupled with the appropriately protected amine with standard coupling agents, such as chloroformates in the presence of a mild base, which then can cyclize to form the desired cyclized product. For example, the coupling and cyclization can be achieved with isobutylchloroformate and N-methyl morpholine in one pot.

These reactions can be accomplished at any temperature that achieves the desired result, i.e., that is suitable for the reaction to proceed at an acceptable rate without promoting decomposition or excessive side products. The preferred temperature for the coupling is −15° C. to room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably THF.

Lastly, the optional protection of the functional groups on nitrogen can be deprotected using standard methods.

2. Preparation of Subsequent Coupled Product

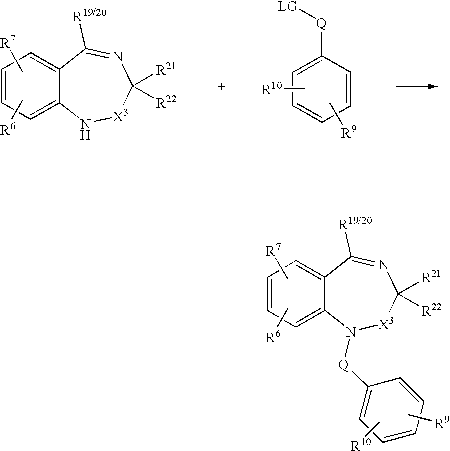

The coupling of the secondary amine with an aryl moiety can be achieved with a base. For example the coupling can be achieved with sodium hydride and extracted from water.

This reaction can be accomplished at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably DMF.

C.5 Alternative Process for Manufacturing Compounds of the General Structure III, Fourth Embodiment

1. Preparation of Functionalized Nitrobenzene Starting Material

The key starting material for this process is an appropriately substituted nitrobenzene; the nitrobenzene can be purchased or can be prepared by any known means including standard reduction and coupling techniques. In one embodiment, the particular nitrobenzene is prepared from a selected aniline, for example by coupling the aniline according to the following protocol.

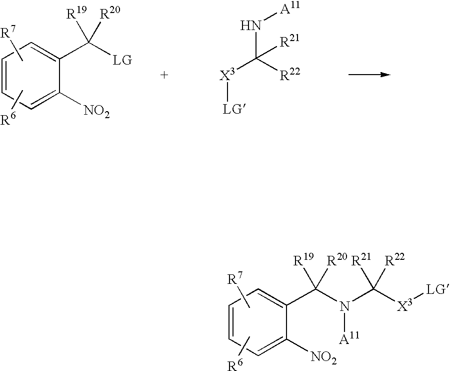

The nitrobenzene can be coupled with the appropriate amine in the presence of a mild base, preferably triethylamine, with an aqueous work up. Importantly, the leaving group on the amine should not be as good as the leaving group on the nitrobenzene to prevent undesired polymerization.

These reaction can be accomplished at any temperature that achieves the desired result, i.e., that is suitable for the reaction to proceed at an acceptable rate without promoting decomposition or excessive side products. The preferred temperature for the coupling is room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably dichloromethane.

2. Preparation of Subsequent Cyclized Product

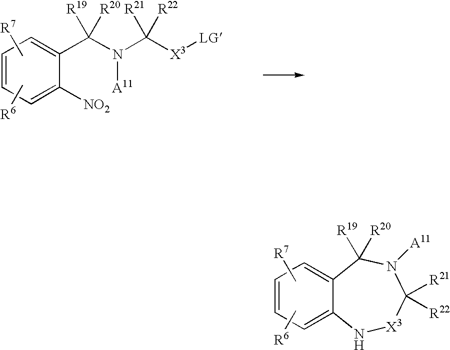

The cyclization of the nitrobenzene can be achieved by any known means including standard reduction and coupling techniques. In one embodiment, the particular cyclized product is prepared from reduction of the nitrobenzene to the aniline using standard reducing agents, such as pressurized hydrogen gas (30 psi) over Raney Nickel. The subsequent intramolecular cyclization can be achieved with acid catalysis, preferably with warm aqueous hydrochloric acid.

These reactions can be accomplished at any temperature that achieves the desired result, i.e., that is suitable for the reaction to proceed at an acceptable rate without promoting decomposition or excessive side products. The preferred temperature for the reduction is room temperature. The preferred temperature for the cyclization is 25-80° C.

Any reaction solvent can be selected that can achieve the necessary temperature, can solubilize the reaction components and inert to the reagents. Non-limiting examples are any protic solvent including, but not limited to methanol, ethanol and water. The preferred solvent for the reduction is methanol. The preferred solvent for the cyclization is water.

3. Preparation of Coupled Product

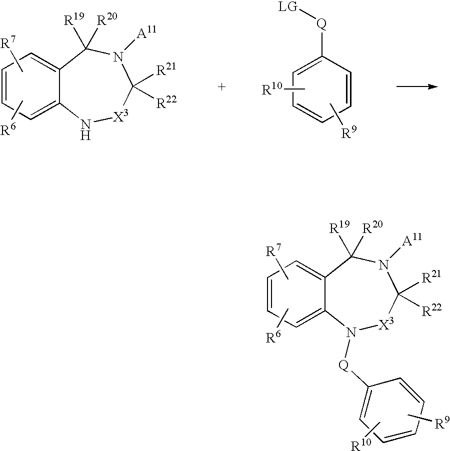

The coupling of the secondary amine with an aryl moiety can be achieved with a base. For example the coupling can be achieved with sodium hydride and extracted from water.

This reaction can be accomplished at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Non-limiting examples are any aprotic solvent including, but not limited to the alkyl solvents, such as hexane and cyclohexane, toluene, acetone, ethyl acetate, dithianes, tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably DMF.

### EXAMPLES

Examples of the compounds, illustrated in Schemes 1 to 20, which may be prepared according to the present invention.

The following working examples provide a further understanding of the method of the present invention. These examples are of illustrative purpose, and are not meant to limit the scope of the invention. Equivalent, similar or suitable solvents, reagents or reaction conditions may be substituted for those particular solvents, reagents or reaction conditions described herein without departing form the general scope of the method of synthesis.

Melting points were taken on a Thomas Hoover capillary melting point apparatus and are not corrected. NMR spectra were recorded either at 300 MHz on a Mercury-300 or at 400 MHz on a Varion INOVA 400 spectrometer. Spectra taken in deuterated chloroform (CDCl₃) used residual chloroform (¹H NMR d 7.26 ppm) as the internal standard. Spectra taken in dimethylsulfoxide-d6 (DMSO-d6) used residual DMSO (¹H NMR d 2.50 ppm) as the internal standard. Mass spectra were obtained on either a VG 70-S Nier Johnson or a JEOL Mass Spectrometer, purchased through NIH and NSF as shared instruments. All reactions were performed under dry nitrogen except for 1, 7, 8, and 16.

### Example 1

Example for Compounds of Type I

The approach towards the synthesis of RL 1019 is shown in Scheme 1. The amino compound 1 was synthesized simply by treating p-phenylenediamine with benzoic acid in the presence of DCC. Then, treatment of camphorsulfonylchloride with 1 in the presence of triethylamine gave the desired compound in good yield.

Synthesis of 1: To a solution of p-phenylenediamine (1 eq) in THF at room temperature, benzoic acid (1.1 eq) was added followed by DCC (1.1 eq). The mixture was poured into water after stirring for 12 h and extracted with Ethyl acetate. The combined organics were washed with brine and dried over Na₂SO₄. Flash column chromatography yielded 1.

1 (¹H NMR, CDCl₃): 9.87 (br s, 1H), 7.9 (d, 2H, J=8.1 Hz), 7.5 (m, 4H), 6.5 (d, 3H, J=8.1 Hz), 4.9 (br s, 2H).

Synthesis of RL1019: To a solution of S-camphorsulfonyl chloride (1 eq) in dichloromethane at room temperature, diisopropylethyl amine (1.5 eq) was added followed by 1. After stirring for 3 h at room temperature, the reaction mixture was poured into water and extracted with dichloromethane. The combined organics were washed with brine and dried over Na₂SO₄. Flash chromatography of the crude material yielded RL1019.

RL1019 (¹H NMR, CDCl₃): 7.95 (m, 4H), 7.65 (d, 1H, J=9 Hz), 7.52 (m, 3H), 7.32 (d, 1H, J=9 Hz), 3.35 (d, 1H, J=15.3 Hz), 2.8 (d, 1H, J=15.3 Hz), 2.5 (m, 1H), 2.24-1.95 (m, 5H), 1.49 (m, 1H), 0.95 (s, 3H), 0.87 (s, 3H).

The synthesis of RL1001 is shown in Scheme 2. The synthesis of 3 was from the commercially available benzoic acid which was converted into its benzoyl chloride by reaction with thionyl chloride. Then, without purification, reacted with t-butylamine to form the benzamide. Finally, the nitro group was reduced with hydrogen and Pd catalysis. The reaction of aniline 3 with (S)-camphorsulfonylchloride in the presence of triethylamine gave the desired final product, RL1001.

Synthesis of 2 (3-methoxy-4-nitro-t-butylcarboxamidoaniline): A mixture of 3-methoxy-4-nitrobenzoic acid (2.16 g, 11 mmol) in thionyl chloride (10 mL) was refluxed for 2.5 hours. Then the thionyl chloride was removed by evaporation to yield a light yellow solid. This was added portionwise to a solution of triethylamine (1.81 mL, 1.2 equiv) and t-butylamine (1.38 mL, 1.2 equiv) in CH₂Cl₂ (20 mL) at 0° C. and allowed to stir overnight. The triethylamine hydrochloride was filtered and rinsed with CH₂Cl₂. The combined CH₂Cl₂ was washed with H₂O and 5% HCl, then dried (MgSO₄) and evaporated. The remaining oil was recrystallized (EtOAc/Hex) to give 2 (2.7 g, 97%) as water-white needles.

2 (¹H NMR, CDCl₃): 7.8 (d, 1H), 7.6 (s, 1H), 7.2 (d, 1H), 6.0 (bs, NH), 4.0 (s, 3H), 1.5 (s, 9H).

Synthesis of 3 (2-Methoxy-4-t-butylcarboxamidoaniline): To a solution of to 2, 3-methoxy-4-nitro-t-butylcarboxamidoaniline (2.07 g, 8.2 mmol) in 95% EtOH (20 mL) and cyclohexane (5 mL) was added 10% Pd on carbon (600 mg) and the mixture was shaken under an atmosphere of H₂ (30 psi) for 2 hours. The mixture was filtered through Celite and evaporated. The resulting solid furnished compound 3 (1.77 g, 97%) after recrystallization (Hex:EtOAc).

Synthesis of RL1001, (S)-Camphor (2-methoxy-4-t-butylcarboxamido-anilide): To a solution of 3 (200 mg, 0.9 mmol) and triethylamine (0.138 mL, 0.1 mmol, 1.1 equiv) in CH₂Cl₂ at 0° C. was added (1S)-(+)-camphorsulfonyl chloride (226 mg, 1 equiv) in one portion. After 10 hours, the mixture was washed with H₂O, the organic phase was separated, dried and evaporated. The remaining residue was chromatographed (SiO₂) eluting with CH₂Cl₂/MeOH to furnish the title compound (295 mg, 75%).

RL1001 (¹H NMR, CDCl₃): 7.61 (d, 1H), 7.59 (s, 1H), 7.5 (s, 1H), 7.19 (d, 1H), 5.9 (s, 1H), 3.95 (s, 3H), 3.5 (d, 1H), 2.9 (d, 1H), 2.4-1.82 (m, 4H), 1.5 (s, 9H), 1.2 (m, 1H), 1.1 (s, 3H), 0.99 (m, 1H), 0.85 (s, 3H).

The approach towards the synthesis of RL1033 and RL1034 is shown in Scheme 3. Treatment of R-camphorsulfonylchloride with N-o-tolyl-piperazine (4) in the presence of diisopropylethylamine yielded the corresponding sulfonamide (5). The conversion of the ketone moiety to an amino group was achieved through formation of the oxime (6) followed by reduction using 75 psi of H₂ and catalytic Raney Ni. The reduction reaction gave a mixture of both exo- and endo amine in the ratio of 1:3.

Synthesis of 5: To a stirred, 0° C. solution of 1-(2-methylphenyl)piperazine hydrochloride (1 eq) and (−)-10-camphorsulfonyl chloride (1.1 eq) in CHCl₃ was added diisopropylethyl amine (2.2 eq) dropwise over 5 min. The solution was stirred at 0° C. for 1 h and then at ambient temperature for 4 h. The solution was washed with 5% aqueous HCl water, and saturated aqueous NaHCO₃. The organic phase was dried (Na₂SO₄) and filtered, and the solvent was removed under reduced pressure. Flash chromatography using 1:4 ethylacetate:hexanes yielded the title compound.

5 (¹H NMR, CDCl₃): 7.2 (m, 2H), 7.0 (m, 2H), 3.45 (m, 4H), 3.40 (d, 1H, J=15.4 Hz), 3.0 (m, 4H), 2.57 (d, 1H, J=15.4 Hz), 2.4 (m, 1H), 2.3 (s, 3H), 2.1 (m, 2H), 1.96 (d, 1H, J=13.7 Hz), 1.6 (m, 2H), 1.44 (m, 1H), 1.2 (s, 3H), 0.94 (s, 3H).

Synthesis of 6: To a stirred solution of 5 (1 eq) in pyridine was added hydroxylamine hydrochloride. The solution was heated to 70° C. and stirred for 20 h. The solvent was removed under reduced pressure and the residue was dissolved in CHCl₃ and washed with NaHCO₃, water, and 5% aqueous HCl. The organic phase was dried Na₂SO₄ and filtered, and the solvent was removed under reduced pressure. The title compound was purified using column chromatography (1:3 ethyl acetate: hexanes) in good yield. 6 (¹H NMR, CDCl₃): 8.2 (br s, 1H), 7.2 (m, 2H), 7.0 (m, 2H), 3.4 (m, 5H), 3.0 (m, 4H), 2.9 (d, 1H, J=14.4 Hz), 2.6-2.4 (m, 2H), 2.3 (s, 3H), 2.1 (d, 1H, J=18 Hz), 2.0-1.7 (m, 3H), 1.3 (m, 1H), 1.1 (s, 3H), 0.87 (s, 3H).

Synthesis of RL1033 and RL1034: Compound 6 and Raney Ni (Fluka brand) in 2-methoxy-ethanol were shaken on a Parr apparatus under 60 psi of hydrogen for 36 h. TLC indicated complete consumption of the oxime and an approximately 1:3 mixture of exo and endo amines respectively. The mixture was cautiously filtered through Celite, and the filter cake was washed with EtOH and EtOAc and the solvent was removed under reduced pressure. The dried solid was purified using a 98:2 to 95:5 A:B gradient elution (A=CHCl₃, B=95:5 MeOH: NH₄OH).

RL1033 (¹H NMR, CDCl₃): 7.2 (m, 2H), 7.0 (m, 2H), 3.5-3.4 (m, 5H), 3.0 (m, 4H), 2.9 (m, 2H), 2.4-2.2 (m, 2H), 2.3 (s, 3H), 1.8-1.6 (m, 3H), 1.3 (m, 1H), 0.96 (s, 3H), 0.94 (s, 3H), 0.8 (dd, 1H, J=3.9 Hz, 13.2 Hz).

RL1034 (¹H NMR, CDCl₃): 7.2 (m, 2H), 7.0 (m, 2H), 3.5 (m, 5H), 3.4 (m, 1H), 3.0 (m, 4H), 2.7 (d, 1H, J=13.2 Hz), 2.31 (s, 3H), 1.8-1.5 (m, 8H), 1.2 (m, 1H), 1.04 (s, 3H), 0.85 (s, 3H).

For compounds RL1035-RL1041 highlighted in Scheme 4, the endo amine was coupled to N-BOC protected amino acids (a, b, c, d, e, f or g) using EDC, HOBT and diisopropylethyl amine. These N-BOC protected amino acids could be obtained either commercially or could be synthesized in one step from the corresponding amino acids using BOC-ON. After the coupling reaction, deprotection of the respective compounds with TFA in dichloromethane yielded RL1035-RL1041.

The N-BOC amino acids b, c, d, and f were purchased commercially. N-BOC amino acid a was prepared according to a known literature procedure (J. Org. Chem. 55, 3194 (1990)). The same procedure was utilized for the synthesis of N-BOC amino acids e and g.

e (¹H NMR, CDCl₃): 10.1 (br s, 1H), 4.7 (br s, 1H), 3.9 (br t, 2H, J 7.5 Hz), 2.6 (br s, 2H), 1.4 (s, 9H).

g (¹H NMR, CDCl₃): 4.0 (br d, 2H), 2.8 (br t, 2H), 2.5 (m, 1H), 1.9 (br m, 2H), 1.6 (m, 2H), 1.5 (s, 9H)

Synthesis of Compounds of the General Structure 7: To a stirred solution of RL1033 (1 eq), N-BOC amino acid (1.1 eq), HOBT (1.1 eq), and EDC (1.25 eq) in DMF was added DIEA (2 eq) dropwise over a period of 5 min. After the reaction was stirred for 14 h, EtOAc was added to the reaction mixture and the organic layer was washed with 5% aqueous citric acid, water, and saturated aqueous NaHCO₃ and brine. The organic phase was dried over Na₂SO₄ and filtered. The solvent was removed under reduced pressure and the residue was purified using EtOAc:Hexanes.

Synthesis of RL1035-RL1041: To a stirred solution of the coupling product (0.25 g) in dichloromethane was added TFA (1 mL). After 3 h, the solvents were removed under reduced pressure. The residue was dissolved in EtOAc and washed with aqueous NaHCO₃. The organic phase was dried (Na₂SO₄) and filtered, and the solvent was removed under reduced pressure. Column chromatography using 10% MeOH in dichloromethane yielded the requisite deprotected products in good yield.

RL1035 (¹H NMR, CDCl₃): 7.77 (br d, 1H, J=9.6 Hz), 7.20 (m, 2H), 7.02 (m, 2H), 4.48 (m, 1H), 3.38 (m, 5H), 3.23 (d, 1H, J=13.8 Hz), 2.92 (m, 4H), 2.82 (d, 1H,J=13.8 Hz), 2.4 (m, 1H), 2.2 (s, 3H), 2.1 (m, 1H), 2.0- 1.7 (m, 3H), 1.6-1.48 (m, 4H), 1.35(m, 1H), 1.04 (s, 3H), 0.98 (s, 3H), 0.95 (m, 4H).

RL1036 (¹H NMR, CDCl₃): 8.0 (d, 1H, J=8.7 Hz), 7.2 (m, 2H), 7.0 (m, 2H), 4.4 (m, 1H), 4.05 (dd, 1H, J=3.3 Hz, 11.1 Hz), 3.6 (dd, 1H, J=6.3 Hz, 11.4 Hz), 3.5 (m, 6H), 3.0 (m, 4H), 2.9 (d, 1H, J=13.8 Hz), 2.5-1.9 (m, 7H), 2.32 (s, 3H), 1.8 (m, 1H), 1.4 (m, 1H), 1.03 (s, 3H), 0.97 (s, 3H), 0.9 (m,1H).

RL1037 (¹H NMR, CDCl₃): 7.7 (br d, 1H, J=9.3 Hz), 7.34-7.15 (m, 7H), 7.0 (m, 2H), 4.51 (m, 1H), 3.7 (dd, 1H, J=3.6 Hz, 10.5 Hz), 3.4-3.3 (m, 4H), 3.28 (d, 1H, J=13.8 Hz), 3.0 (m, 4H), 2.8 (d, 1H, J=13.8 Hz), 2.62 (dd, 1H, J=10.2 Hz, 13.8 Hz), 2.45 (m, 1H), 2.31 (s, 3H), 2.1-1.8 (m, 3H), 1.72 (m, 1H), 1.5 (br s, 3H), 1.45 (m, 1H), 1.04 (s, 3H), 0.97 (s, 3H), 0.95 (m, 1H).

RL1038 (¹H NMR, CDCl₃): 7.6 (br d, 1H, J=8.4 Hz), 7.2 (m, 2H), 7.05 (m, 2H), 4.5 (m, 1H), 3.4 (m, 4H), 3.2 (m, 2H), 3.0 (m, 4H), 2.84 (d, 1H, J=14 Hz) 2.5 (s, 3H), 2.45 (m, 1H), 2.3 (s, 3H), 2.05 (m, 2H), 1.8 (m, 3H), 1.4 (m, 2H), 1.05 (s, 3H), 0.97 (s, 3H), 0.85 (m,1H).

RL1039 (¹H NMR, CDCl₃): 8.1 (br d, 1H), 7.2 (m, 2H), 7.03 (m, 2H), 4.36 (m, 2H), 3.8 (m,1H), 3.4 (m, 5H), 3.35 (d, 1H, J=14.1 Hz), 3.0 (m, 5H), 2.9 (d, 1H, J=14.1 Hz), 2.6 (m, 1H), 2.4 (m, 1H), 2.3 (s, 3H), 2.2 (m, 1H), 1.8 (br m, 3H), 1.7 (m, 1H), 1.4 (m, 1H), 1.06 (s, 3H), 0.99 (s, 3H), 0.94 (m, 1H).

RL1040 (¹H NMR, CDCl₃): 8.0 (d, 1H, J=9.3 Hz), 7.2 (m, 2H), 7.0 (m, 2H), 4.4 (m, 1H), 3.8 (m, 1H), 3.4 (m, 4H), 3.3 (d, 1H, J=13.8 Hz), 3.0 (m, 6H), 2.9 (d, 1H, J=13.8 Hz), 2.48 (m, 1H), 2.3 (s, 3H), 2.1-1.7 (m, 9H), 1.4 (m, 1H), 1.02 (s, 3H), 0.97 (s, 3H), 0.8 (m, 1H).

RL1041 (¹H NMR, CDCl₃): 7.2 (m, 2H), 7.0 (m, 2H), 6.5 (d, 1H, J=6 Hz), 4.2 (m, 1H), 3.4 (m, 4H), 3.3 (m, 2H), 3.1 (d, 1H, J=14.1 Hz), 3.0 (m, 4H), 2.9 (d, 1H, J=14.1 Hz), 2.7 (m, 2H), 2.5 (m, 3H), 2.3 (s, 3H), 2.0-1.6 (m, 8H), 1.4 (m, 1H), 1.02 (s, 3H), 0.97 (s, 3H), 0.86 (m, 1H).

For compounds RL1042-RL1045, the endo and exo amines were first treated with chloroacetylchloride and the corresponding derivatives (8 and 9, respectively) were subsequently reacted either with morpholine or N-methyl piperazine (Scheme 5).

Synthesis of 8 and 9: To a solution of the amine (endo or exo, 1 eq) in dichloromethane at −78° C. was added triethylamine (2 eq) followed by chloroacetyl chloride (1.1 eq). The reaction mixture was stirred for 30 min and quenched with water and extracted with methylene chloride. The organic layer was washed with brine, dried over Na₂SO₄ and the solvents were removed under reduced pressure. Flash chromatography using 2:3 EtOAc:Hexanes yielded the title compound in good yield.

8 (¹H NMR, CDCl₃): 7.2 (m, 2H), 4.4 (m, 2H), 4.1 (m, 2H), 3.4 (m, 4H), 3.1 (d, 1H, J=14.1 Hz), 3.0 (m, 4H), 2.9 (d, 1H, J=14.1 Hz), 2.5 (m, 1H), 2.3 (s, 3H), 2.1 (m, 2H), 1.9 (m, 1H), 1.8 (m, 1H), 1.4 (m, 1H), 1.04 (s, 3H), 0.97 (s, 3H), 0.87 (m, 1H).

9 (¹H NMR, CDCl₃): 7.2 (m, 2H), 7.02 (m, 2H), 6.9 (d, 1H, J=6.6 Hz), 4.4 (m, 1H), 4.1 (s, 2H), 3.4 (m, 4H), 3.1 (d, 1H, J=14.1 Hz), 3.0 (m, 4H), 2.9 (d, 1H, J=14.1 Hz), 2.3 (s, 3H), 2.1 (m, 2H), 1.9 (m, 4H), 1.4 (m, 1H), 1.05 (s, 3H), 0.98 (s, 3H).

Synthesis of RL1042-RL1045: To a solution of the chloride (exo or endo, 1 eq) in 1,2-dichloroethane, morpholine or N-methylpiperazine (3 eq) was added and refluxed for 10 h. The mixture was cooled and water was added and extracted with dichloromethane. The combined organic layers were washed with brine and dried over NaSO₄. The solvent was removed under reduced pressure. Flash chromatography with 5% MeOH in dichloromethane yielded the desired products in good yield.

RL1042 (¹H NMR, CDCl₃): 7.8 (d, 1H, J=8.7 Hz), 7.2 (m, 2H), 7.02 (m, 2H), 4.41 (m, 1H), 3.38 (m, 4H), 3.2 (d, 1H, J=13.8 Hz), 3.1-2.95 (m, 7H), 2.9 (d, 1H, J=13.8 Hz), 2.7 (br s, 2H), 2.5 (br m, 4H), 2.33 (s, 3H), 2.30 (s, 3H), 2.1-1.9 (m, 4H), 1.78 (m, 1H), 1.36 (m, 2H), 1.03 (s, 3H), 0.97 (s, 3H), 0.94 (m, 1H).

RL1043 (¹H NMR, CDCl₃): 7.8 (br d, 1H, J=8.6 Hz), 7.2 (m, 2H), 7.01 (m, 2H), 4.4 (m, 1H), 3.8 (m, 4H), 3.4 (m, 4H), 3.2 (d, 1H, J=14 Hz), 3.0 (m, 6H), 2.85 (d, 1H, J=14 Hz), 2.6 (m, 2H), 2.5 (m, 3H), 2.31 (s, 3H), 2.0 (m, 3H), 1.8 (m, 1H), 1.4 (m, 1H), 1.04 (s, 3H), 0.98 (s, 3H), 0.9 (m, 1H).

RL1044 (¹H NMR, CDCl₃): 7.8 (br d, 1H, J=8.4 Hz), 7.2 (m, 2H), 7.0 (m, 2H), 4.3 (m, 1H), 3.7 (m, 6H), 3.4 (m, 4H), 3.1 (d, 1H, J=14.2 Hz), 3.0 (m, 5H), 2.9 (d, 1H, J=14.2 Hz), 2.6 (m, 2H), 2.5 (m, 2H), 2.3 (s, 3H), 2.04 (m, 2H), 1.8-1.6 (m, 3H), 1.35 (m, 1H), 1.05 (s, 3H), 0.97 (s, 3H).

RL1045 (1H NMR, CDCl₃): 7.60 (d, 1H, J=9 Hz), 7.2 (m, 2H), 7.0 (m, 2H), 4.25 (m, 1H), 3.4 (m, 4H), 3.1 (d, 1H, J=14.1 Hz), 3.0 (m, 6H), 2.9 (d, 1H, J=14.1 Hz), 2.6 (br s, 4H), 2.4 (br s, 3H), 2.3 (s, 3H), 2.0 (m, 3H), 1.9-1.6 (m, 6H), 1.4 (m, 1H), 1.03 (s, 3H), 0.97 (s, 3H), 0.87 (m, 1H).

### Example 2

Example for Compounds of Type II

The synthesis of RL1002, RL1003 and RL1004 is shown in Scheme 6. The sulfonylhydrazides 13, 14 and 15 were prepared as shown in Scheme 6, by reaction of sulfonyl chloride 10, 11 or 12 with hydrazine. Then sulfonylhydrazides 13-15 and (1R)-camphor were condensed with acid catalysis.

Synthesis of 10, 4-tert-Butylcarbamoyl-benzenesulfonyl chloride: To 4-sulfobenzoic acid potassium salt (1 eq) was added phosphorus pentachloride (3 eq) and the reaction mixture was heated to 100° C. for three hours while occassionally stirring with a glass rod. The reaction mixture was quenched with ice water (0.5M) and filtered through a fritted funnel to obtain 4-chlorosulfonyl-benzoyl chloride. ¹H NMR (400 MHz, DMSO) δ 7.85 (m, 4H). Then, to a solution of 4-chlorosulfonyl-benzoyl chloride (1 eq) in methylene chloride (0.1M) at −78° C. was added the mixture of triethylamine (1.2 eq) and t-butylamine (1 eq) dropwise. The reaction was allowed to warm to −50° C. The reaction was quenched after half an hour with water (0.1M) and extracted with methylene chloride (3×). The combined organic layers were washed with brine and dried with magnesium sulfate. Purification by column chromatography using methylene chloride yielded the title compound 10 (60%). ¹H NMR (400 MHz, CDCl₃): δ 1.492 (s, 9H), δ 5.947 (br s, 1H), δ 8.017 (dd, 4H, J=63Hz, J=8.8Hz).

Synthesis of 11, 4-tert-Butylcarbamoyl-2-methoxy-benzenesulfonyl chloride: Sulfuric acid with 3% SO₃ (4.8M) was added to 3-hydroxybenzoic acid (1.0 eq) and the mixture was heated for three hours at 90° C. Water (1.5M) was added to quench the reaction and to dissolve all the compound. The mixture was allowed to cool to room temperature. Potassium hydroxide (25% by weight) was then added dropwise. Recrystallization from water yielded 3-hydroxy-4-sulfo-benzoic acid potassium salt in 90% yield. Subsequently, to a solution of the hydroxy-potassium salt (1.0 eq) in water (2.87M) was added potassium hydroxide to attain pH of 14. The mixture cooled to 0° C. and dimethyl sulfate (0.52 eq) was added dropwise. The mixture was warmed to room temperature. Five more additions of two equivalents of potassium hydroxide and 1 equivalent of dimethyl sulfate were added one addition every thirty minutes to push the reaction to completion. The pH of the reaction mixture was adjusted to 7 with concentrated sulfuric acid and then adjusted to pH 1 concentrated hydrochloric acid. The solid that precipitated out was filtered through a fritted funnel. Recrystallization from water yielded 3-methoxy-4-sulfo-benzoic acid potassium salt (18% Yield). 1H NMR (400 MHz, CDCl₃): δ 3.359 (br s), δ 3.812 (s, 3H), δ 7.454 (m, 2H), 6 7.775 (d, 1H, J=8Hz). Then, the methoxy-potassium salt (1.0 eq) and phosphorus pentachloride (2.6 eq) were stirred with a glass rod at 100° C. for two hours. The reaction mixture was quenched with ice water (0.5M) and filtered through a fritted funnel. The solid obtained was recrystallized from carbon tetrachloride to yield 4-chlorosulfonyl-3-methoxy-benzoyl chloride (28% Yield). ¹H NMR (400 MHz, CDCl₃): δ 4.157 (s, 3H), δ 7.780 (d, 1H, J=1.6Hz), δ 7.873 (dd, 1H, J=8.4Hz, J=1.6Hz), δ 8.122 (d, 1H, J=8.4Hz). Lastly, to a solution of the benzoyl chloride (1.0 eq) in methylene chloride (0.1M) at −78° C. was added a mixture of tert-butylamine (1.0 eq) and triethylamine (1.2 eq) dropwise. The reaction mixture was allowed to warm to room temperature and react for 2 hours. The reaction mixture was then quenched with water (0.15M) and extracted with methylene chloride (3×). The combined organics were washed with brine and dried over magnesium sulfate. The solvent was evaporated under reduced pressure. Purification by column chromatography using methylene chloride yielded the desired product 11 (69%). ¹H NMR (400 MHz, CDCl₃): δ 1.489 (s, 9H), δ 4.113 (s, 3H), δ 5.975 (s, 1H), δ 7.230 (d, 1H, J=8.4Hz), δ 7.614 (d, 1H, J=1.2Hz), δ 7.976 (d, 1H, J=8.4Hz).

Synthesis of 13, 4-t-Butylcarboxamidobenzenesulfonhydrazide: To a solution of 10 (223 mg, 0.66 mmol) dissolved in CH₂Cl₂ (5 mL) at 0° C. was added hydrazine (47 mg, 1.5 mmol, 2 equiv). After 20 min, the white solid is filtered and washed with water. The product was then air dried (173 mg, 76%). Was made in similar fashion to 14 from 10. (¹H NMR, CDCl₃): 7.9 (dd, 4H), 6.0 (s, 1H), 5.7 (s, 1H), 3.6 (s, 2H), 1.5 (s, 9H).

Synthesis of 14, 2-Methoxy-4-t-butylcarboxamidobenzenesulfonhydrazide: Was made in similar fashion to 13 from 11 (153 mg, 76%). (¹H NMR, CDCl₃): 8.0 (dd, 4H), 7.5 (s, 1H), 7.3 (s, 1H), 6.1 (s, NH), 4.0 (s, 3H), 2.7 (bs, 2H), 1.5 (s, 9H).

RL1002, (R)-Camphor (2-methoxy-4-t-butylcarboxamidobenzene)sulfonylhydrazone: To a solution of (1R)-(+)-camphor (300 mg, 1.9 mmol) in toluene (10 mL) was added 13 (367 mg, 1.9 mmol) and the mixture was refluxed for 18 hours with water removed by a Dean-Stark trap. The solvent was evaporated and the residue was partitioned between H₂O and CH₂Cl₂. The organic phase was evaporated and the remaining solid was recrystallized from 50% H₂O/EtOH to yield the title compound (370 mg, 58%). (¹H NMR, CDCl₃): 8.0 (d, 1H), 7.5 (s, 1H), 7.4 (s, NH), 7.21 (s, 1H), 6.1 (s, NH), 4.0 (s, 3H), 1.95 (t, 1H), 1.9-1.7 (m, 2H), 1.65-1.5 (m, 4H), 1.45 (s, 9H), 1.25-1.1 (m, 2H), 0.85 (s, 3H), 0.7 (s, 3H), 0.55 (s, 3H).

RL1003, (R)-Camphor 4-t-butylcarboxamidobenzenesulfonylhydrazone: Was prepared in the similar manner as RL1002 using 14. (¹H NMR, CDCl₃): 8.0 (d, 2H), 7.8 (d, 2H), 6.0 (bs, 1H), 2.4-1.6 (m, 10H), 1.5 (s, 9H), 1.4-1.1 (m, 5H), 0.9 (dd, 6H), 0.55 (s, 3H).

RL1004, (R)-Camphor p-tosylhydrazone: Was prepared in the similar manner as RL1002 using 15. (¹H NMR (CDCl₃): 7.7 (d, 1H), 7.5 (d, 1H), 7.2 (d, 1H), 7.15 (d, 1H), 2.5-0.6 (m, 19H).

### Example 3

Example for Compounds of Type III.1

The synthesis of RL1005 and RL1006 is shown in Scheme 7. The benzenesulfonanilide were prepared by reaction of sulfonyl chloride 11 or 16 with 4-ethoxyaniline (p-phenetidine) in the presence of triethylamine.

Synthesis of RL1005, 2-Methoxy-4-t-butylcarboxamidobenzenesulfon-(4-ethoxy-anilide): To a solution of p-phenetidine (45 mg, 0.33 mmol) and triethylamine (0.396 mmol, 1.2 equiv, 55 μL) in CH₂Cl₂ (3 mL) at 0° C. was 11 (100 mg, 1 equiv) in one portion. After 8 hours, H₂O was added and the CH₂Cl₂ was separated, dried (MgSO₄) and evaporated. The remaining residue was chromatographed (SiO₂; CH₂Cl₂:MeOH) to furnish the title compound as a white solid (100 mg, 75%).

RL1005 (¹H NMR, CDCl₃): 7.7 (d, 1H), 7.55 (s, 1H), 7.05 (d, 1H), 6.9 (d, 2H), 6.79 (s, 1H), 6.65 (d, 2H), 5.99 (s, 1H), 4.1 (s, 3H), 3.9 (q, 2H), 1.45 (s, 9H), 1.3 (t, 3H).

Synthesis of 16 (4-trimethylacetylaminobenzenesulfonyl chloride): To a solution of aniline (2.5 g, 26.8 mmol) and triethylamine (4.48 mL, 1.1 equiv) in CH₂Cl₂ (20 mL) cooled to 0° C. was added dropwise with vigorous stirring trimethylacetyl chloride (3.3 mL, 1 equiv). After addition completed, reaction was allowed to warn to room temperature and stirred 1 hour. Solids were filtered and the filtrate was washed with water, dried (MgSO₄) and evaporated. The trimethylacetylamide (3.75 g, 79%) was purified by a single recrystallization (hexanes:EtOAc). To chlorosulfonic acid (5 equiv, 1.87 mL) cooled to 0° C. was added trimethylacetylamide (1 g, 5.6 mmol) portionwise. After addition complete, reaction was warmed to 15° C. for 1 hr then 60° C. for 2 hr. The reaction mixture was cooled to 0° C. and ice and water (10 g) was added with vigorous stirring. The yellow solid 16 (1.2 g, 77%) was filtered and sucked dry.

RL1006, N-Trimethylacetyl-sulfanilic acid-(4-ethoxy-anilide): Was made in similar fashion to RL1005 from 16. (¹H NMR, CDCl₃): 7.6 (s, 2H), 7.5 (s, 1H), 6.95 (d, 1H), 6.75 (d, 2H), 6.62 (d, 1H), 6.5 (s, 1H), 3.95 (q, 2H), 1.39 (t, 3H), 1.25 (s, 9H).

The synthesis of RL1027 and 1028 is shown in Scheme 8. Alkylation of p-phenetidine with 4-(2-chloroethyl)-morpholine went smoothly in the presence of sodium iodide. The diamine 17 was then sulfonlylated with benzenesulfonyl chloride 18 or 11 to furnish the target compounds RL1027 and 1028, respectively.

Synthesis of 17: To a solution of p-phenetidine (200 mg, 1.46 mmol), K₂CO₃ (200 mg, 1 equiv) and sodium iodide (219 mg, 1 equiv) in dioxane (5 mL) was added 4-(2-chloroethyl)morpholine and the mixture was refluxed. After 8 hours, the reaction was cooled and the solvent was evaporated and the residue was partitioned between H₂O and CH₂Cl₂. The organic layer was separated, dried (MgSO₄) and evaporated. The resulting oil was chromatographed (Al₂O₃; CH₂Cl₂) to yield 17. (200 mg, 55%). (¹H NMR, CDCl₃): 6.8 (d, 2H), 6.6 (d, 2H), 4.05 (s, NH), 3.95 (q, 2H), 3.7 (t, 4H), 3.1 (t, 2H), 2.6 (t, 2H), 2.5 (m, 4H), 1.4 (t, 3H).

RL1027, N-(2-Morpholin-4-yl-ethyl)-N-(4-ethoxyphenyl)-benzenesulfonamide: To a solution of 17 (100 mg, 0.4 mmol) and triethylamine (60 mg, 1.5 equiv) in CH₂Cl₂ (5 mL) was added benzenesulfonyl chloride, 18, (70 mg, 1 equiv) in one portion. After stirring 8 hours, water was added and the organic layer was separated, dried (MgSO₄) and evaporated. The resulting residue was chromatographed (SiO₂; CH₂Cl₂:MeOH) to yield the title compound (110 mg, 70%) as a slightly orange oil. (¹H NMR, CDCl₃): 7.65-7.42 (m, 5H), 6.95 (d, 2H), 6.8 (d, 2H), 4.0 (q, 2H), 3.65 (m, 6H), 2.4 (m, 6H), 1.4 (t, 3H).

RL1028 4-Phenylcarboxamido-N-(2-morpholin-4-yl-ethyl)-N-(4-ethoxyphenyl)-benzenesulfonamide: Was prepared in a similar fashion as RL1027 from 16. (¹H NMR, CDCl₃): 8.0 (s, NH), 7.9-7.5 (m, 9H), 7.0 (d, 2H), 6.8 (d, 2H), 4.0 (q, 2H), 3.65 (m, 6H), 2.4 (m, 6H), 1.4 (t, 3H).

The approach towards the synthesis of RL1051-RL1053 is shown in Scheme 9. Sulfonylation of 2-aminoacetophenone with 4-nitrobenzenesulfonylchloride and pyridine followed by reduction of the nitro group using 40 psi of H₂ in the presence of Raney Ni yielded the amino derivative (19) in good yield. Treatment of this compound with either pivaloyl chloride or benzoyl chloride furnished the amides RL1051 or RL1052, respectively in good yield. Reduction of RL1052 with NaBH₄ in MeOH yielded RL1053.

Synthesis of 19: To a solution of 2-amino acetophenone (1 eq) in ether was added pyridine (1.5 eq) and the solution cooled to 0° C. 4-Nitrobenzenesulfonyl chloride (1.1 eq) was added and the reaction mixture stirred for four hours at this temperature. The ether was removed under reduced pressure and the solid obtained was triturated with cold MeOH. Filtration of the solid yielded the desired compound in good yield. (¹H NMR, D₆-DMSO): 11.2 (br s, 1H), 8.4 (d, 2H, J=9 Hz), 8.1 (d, 2H, J=9 Hz), 8.0 (d, 1H, J=8.1 Hz), 7.6 (m, 1H), 7.3 (m, 2H), 2,6 (s, 3H).

Synthesis of 20: To the nitro compound obtained above, ethanol was added followed by a little amount of THF to dissolve the compound entirely. This solution was then hydrogenated at 40 psi using catalytic Raney Nickel. The mixture was cautiously filtered through celite, and washed with MeOH. Removal of the solvent under reduced pressure and recrystallization from dichloromethane/hexanes yielded the amino compound (20) in good yield.

Synthesis of RL1051 and RL1052: To a stirred solution of 20 (1 eq) in THF at 0° C. was added triethylamine (2 eq) followed by either pivaloyl chloride or benzoyl chloride (1.1 eq). After 20 minutes at 0° C., the icebath was removed and the mixture stirred at room temperature for 1 h. THF was removed under reduced pressure and the solid obtained was diluted with water and extracted with methylene chloride. The combined organics were washed with brine, dried over Na₂SO₄ and the solvent removed under reduced pressure. Recrystallization from dichloro-methane/hexanes yielded the title compounds in good yield.

RL1051 (¹H NMR, D₆-DMSO): 11.3 (br s, 1H), 9.6 (s, 1H), 7.9 (d, 1H, J=8.1 Hz), 7.8 (d, 2H, J=9 Hz), 7.7 (d, 2H, J=9Hz), 7.5 (m, 1H), 7.4 (d, 1H, J=8.1 Hz), 7.2 (m, 1H), 2.6 (s, 3H), 1.2 (s, 9H).

RL1052 (¹H NMR, D₆-DMSO): 11.3 (br s, 1H), 10.6 (s, 1H), 7.9 (m, 5H), 7.8 (d, 2H, 9 Hz), 7.6-7.4 (m, 5H), 7.1 (m, 1H), 2.6 (s, 3H).

Synthesis of RL1053: Product RL1052 was dissolved in methanol and at 0° C., and NaBH₄ (1.5 eq) was added. After 15 minutes of stirring, the reaction mixture was quenched with water, MeOH was removed under reduced pressure, and extracted with methylene chloride. The combined organics were washed with brine, dried over Na₂SO₄ and the solvent removed under reduced pressure. Purification of the crude material using 5% MeOH in dichloromethane yielded the title compound in moderate yield.

RL1053 (¹H NMR, D₆-DMSO): 10.6 (s, 1H), 9.6 (br s, 1H), 7.9 (m, 4H), 7.7 (d, 2H, J=9 Hz), 7.6 (m, 3H), 7.4 (m, 1H), 7.1 (m, 2H), 6.9 (m, 1H), 5.5 (br s, 1H), 5.0 (q, 1H, J=6.6Hz), 1.2 (d, 3H, J=6.6Hz).

### Example 4

Example for Compounds of Type III.2

The synthesis of RL1015 and RL1016 is shown in Scheme 10. Either 2-fluoronitrobenzene (21) or 2-nitrobenzyl bromide (22) is reacted with 2-hydroxyethyl-1-piperazine to furnish the disubstituted piperazines 23 and 24. These compounds are next protected as silyl ethers and then reduced to anilines 27 and 28 with hydrogen and Raney Ni catalyst. These are then reacted with the sulfonyl chloride, 29, in the presence of triethylamine. Finally, the silyl protection is cleaved with ammonium fluoride to give the target compounds.

Synthesis of 24: To a solution of 1-(2-hydroxyethyl)piperazine (1.06 g, 1.3 eq, 8.15 mmol), K₂CO₃ (1.03 g, 1.2 eq, 7.5 mmol) in 95% EtOH (20 mL) was added 2-fluoronitrobenzene, 21, (1.35 g, 6.25 mmol) in one portion. After stirring 12 hours, the volatiles were evaporated and the mixture was partitioned between H₂O and CH₂Cl₂. The organic phase was separated, dried (MgSO₄) and evaporated. The remaining 1-(2-hydroxyethyl)piperazine was removed by reduced pressure distillation and the residue was chromatographed (SiO₂; CH₂Cl₂:MeOH) to furnish 23 (1.5 g, 90%). (¹H NMR, CDCl₃): 7.7 (d, 1H), 7.4 (t, 1H), 7.1 (d, 1H), 7.0 (t, 1H), 3.0 (bs, 12H), 1.8 (s, 1H).

Synthesis of 25: To a solution of 23 (1.7 g, 6.8 mmol) in THF (20 mL) cooled to 0° C. was added portionwise sodium hydride (178 mg, 1.1 equiv). After stirring 1 hour, t-butylchlorodiphenylsilane (2.23 g, 1.2 equiv) was added dropwise and the cooling bath was removed. After stirring 18 hours, solvent was evaporated and the residue was chromatographed (SiO₂; CH₂Cl₂:MeOH) to furnish 25 (1.19 g, 39%) as a colorless oil. (¹H NMR, CDCl₃): 7.75 (d, 1H), 7.7 (d, 4H), 7.5-7.35 (m, 7H), 7.15 (d, 1H), 7.05 (t, 1H), 3.85 (t, 2H), 3.1 (s, 4H), 2.7 (s, 6H), 1.05 (s, 9H).

Synthesis of 27: A mixture of 25 (420 mg, 0.86 mmol), Raney Nickel (200 mg) in 95% EtOH (50 mL) was shaken under a H₂ atmosphere (30 psi) for 12 hours. The mixture was filtered through Celite and then evaporated. The residue was chromatographed (neutral AL₂O₃; CH₂Cl₂:MeOH) to furnish 27 (340 mg, 86%). (¹H NMR, CDCl₃): 7.7 (d, 4H), 7.4 (m, 6H), 7.05-6.9 (m, 2H), 6.7 (t, 2H), 3.95 (bs, 2H), 3.85 (t, 2H), 2.9 (t, 4H), 2.7 (t, 6H), 1.05 (s, 9H).

Synthesis of 29, 4-Benzoylamino-benzenesulfonyl chloride: To a solution of aniline (1.0 eq) and triethylamine (1.2 eq) in methylene chloride (1M) cooled to 0° C. was added benzoyl chloride (1.0 eq) dropwise. The reaction mixture was allowed half an hour to reach completion. The solvent was evaporated under reduced pressure and the residue was recrystallized from ethanol to give the desired N-Phenyl-benzamide (74%). Melting point: 178-179° C. ¹H NMR (300 MHz, D₆-DMSO): δ 7.101 (t, 1H, J=7.8Hz), δ 7.354 (t, 1H, J=7.5Hz), δ 7.556 (m, 3H), δ 7.782 (d, 2H, J=8.4Hz), δ 7.954 (d, 2H, J=6.9), δ 10.250 (s, 1H). Then, chlorosulfonic acid (3.19M) was added to the benzamide (1.0 eq) and allowed to stir for two hours at 60° C. The reaction was quenched with water (3M) and filtered through a fritted funnel. The residue was recrystallized from acetone and water to give the title compound 29 (50% Yield). ¹H NMR (400 MHz, CDCl₃): δ 7.527 (t, 2H, J=8Hz), δ 7.618 (t, 1H, J=7.2Hz), δ 7.909 (m, 4H), δ 8.030 (d, 2H, J=9.2Hz), δ 8.200 (br s, 1H).

Synthesis of 30: To a solution of 27 (200 mg, 0.465 mmol) and triethylamine (140 mg, 3 equiv) in CH₂Cl₂ (4 mL) was added 29 (138 mg, 1 equiv). After 12 hours, water was added and the organic layer was separated, dried (MgSO₄) and evaporated. The remaining residue was chromatographed (SiO₂; CH₂Cl₂:MeOH) to furnish 30 (260 mg, 81%) as a white solid. (¹H NMR, CDCl₃): 8.0 (s, 1H), 7.9 (s, 1H), 7.8 (m, 5H), 7.7 (d, 7H), 7.6-7.35 (m, 7H), 7.05 (t, 2H), 3.85 (t, 2H), 2.6 (m, 10H), 1.05 (s, 9H).

Synthesis of RL1015, 4-Benzoylamino-[2-(2-hydroxyethyl)-piperazin-1-yl]-phenyl)-benzene-sulfonamide: To a solution of 30 (260 mg, 0.377 mmol) in a mixture of DMF (0.5 mL) and 3 mL (MeOH) was added NH₄F (139 mg, 10 equiv) and the mixture was stirred for 18 hours. All volatiles were evaporated and the mixture was partitioned between CH₂Cl₂ and H₂O. The organic phase was separated, dried (MgSO₄) and evaporated. The residue was chromatographed (SiO₂; CH₂Cl₂:MeOH) to furnish the title compound (130 mg, 76%) as a white solid. (¹H NMR, CDCl₃): 8.0 (bs, 1H), 7.85 (m, 4H), 7.7 (d, 2H), 7.6 (m, 2H), 7.5 (t, 2H), 7.1 (m, 2H), 7.02 (m, 1H), 3.65 (m, 2H), 2.7-2.5 (m, 5H), 1.7 (bs, 5H).

Synthesis of RL1016, 4-Benzoylamino-[2-(2-hydroxyethyl)-piperazin-1-yl-methyl]-phenyl)-benzenesulfonamide was prepared in similar fashion to RL1015 starting with 2-nitrobenzyl bromide, 22. (¹H NMR, CDCl₃): 8.05 (s, NH), 7.85 (d, 2H), 7.75 (q, 4H), 7.6 (m, 1H), 7.5 (t, 3H), 7.21 (m, 1H), 6.95 (m, 2H), 3.65 (m, 7H), 3.25 (s, 2H), 2.65-2.35 (m, 5H).

The synthesis of RL1020 is shown in Scheme 11. Nucleophilic aromatic displacement of 2-fluoronitrobenzene by isonipecotic acid formed carboxylic acid 32. Condensation of this acid with 1-methylpiperazine using 1,1′-carbonyldiimidazole formed the amide 33. Reduction with hydrogen and Raney Ni followed by reaction with sulfonyl chloride 16 in the presence of base furnished the target material.

Synthesis of 32: To a mixture of 21, 1-fluoro-2-nitrobenzene (500 mg, 3.5 mmol), and K₂CO₃ (489 mg, 1 equiv) in DMSO (3 mL) was added isonipecotic acid (460 mg, 1 equiv) and the reaction was heated to 60° C. After 30 min, reaction cooled to room temperature and poured into water and neutralized with conc. HCl. The solid that precipitated was filtered, sucked dry, and recrystallized (Hex:EtOAc:MeOH) to furnish 32 (787 mg, 90%) as a yellow crystal. (¹H NMR, CDCl₃): 7.8 (d, 1H), 7.5 (t, 1H), 7.1 (d, 1H), 7.0 (t, 1H), 3.3 (m, 4H), 2.9 (t, 4H), 2.5 (m, 2H).

Synthesis of 33: To a solution of 1,1′-carbonyldiimidazole (677 mg, 4.18 mmol, 1.1 equiv) in CH₂Cl₂ (20 mL) was added 32 (950 mg, 3.8 mmol) and the mixture was stirred for 2 hours. Then 1-methylpiperazine (456 mg, 1.2 equiv) was added in one portion. After 10 hours, solvent was evaporated and the residue was chromatographed (SiO₂; CH₂Cl₂:MeOH) to 33 (940 mg, 75%) as an orange oil. (¹H NMR, CDCl₃): 6.9 (m, 2H), 6.7 (m, 2H), 4.0 (bs, 2H), 3.65 (bs, 2H), 3.55 (t, 2H), 2.6 (m, 3H), 2.4 (m, 4H), 2.0 (m, 2H), 1.8 (d, 2H).

Synthesis of 34: To a solution of 33 (940 mg, 2.9 mmol) in 95% EtOH (50 mL) was added Raney Ni (200 mg) and the mixture was shaken under an atmosphere of H₂ (30 psi) for 4 hours. Then the solvent was evaporated to a solid which furnished 34 (765 mg, 90%) after recrystallization (Hex:EtOAc:MeOH).

Synthesis of RL1020, 4-t-Butylcarboxamido-(2-[4-(1-methyl-piperazin-4-yl-carboxamido)-piperidin-1-yl]-phenyl)-benzenesulfonamide: To a solution of 34 (200 mg, 0.7 mmol) and triethylamine (85 mg, 1.2 equiv) in CH₂Cl₂ (5 mL) was added 16 (184 mg, 1.1 equiv). After stirring 12 hours, water was added and the organic layer was separated, dried (MgSO₄) and evaporated. The residue was chromatographed (SiO₂; CH₂Cl₂:MeOH) to furnish the title compound (100 mg, 30%) as a white solid. (¹H NMR, CDCl₃): 8.15 (d, 2H), 7.9 (d, 2H), 7.42 (m, 1H), 7.21 (d, 1H), 7.15 (m, 1H), 7.01 (d, 1H), 6.8 (s, NH), 3.61 (bs, 2H), 3.5 (m, 2H), 3.15 (d, 2H), 2.55- 2.35 (m, 8H), 2.3 (s, 3H), 1.8 (bs, 2H), 1.5 (s, 9H).

The synthesis of RL1025 and 1026 are shown in Scheme 12. Starting from 2-fluoro-nitrobenzene, nucleophilic aromatic substitution by piperazine provides the mono-substituted piperazine 35. Alkylation of 35 with 4-(2-chloroethyl)-morpholine went smoothly in presence of sodium iodide. Reduction of the nitro compound followed by sulfonylation with 16 or 29 furnished the target compounds.

Synthesis of 35: To a solution of 21, 1-fluoro-2-nitrobenzene (1 g, 7 mmol), in 95% EtOH was added piperazine (2.4 g, 28.9 mmol, 4 equiv) and the mixture was refluxed. After 12 hours, solvent was evaporated and the residue partitioned between H₂O and CH₂Cl₂. The organic phase was separated, dried (MgSO₄) and evaporated. The residue was then chromatographed (SiO2; CH₂Cl₂:MeOH) to furnish 35 (1.34 g, 91%) as an orange semi-solid. (¹H NMR, CDCl₃): 7.8 (d, 1H), 7.5 (t, 1H), 7.15 (d, 1H), 7.05 (t, 1H), 3.7 (t, 2H), 3.1 (t, 4H), 2.7 (t, 2H), 2.2 (NH.

Synthesis of 36: To a solution of 35 (1.2 g, 5.79 mmol), K₂CO₃ (400 mg, 0.5 equiv) and KI (50 mg) in 95% EtOH (25 mL) was added 4-(2-chloroethyl)morpholine (1.04 g, 1.2 equiv) and the mixture was refluxed. After 3 hours, the reaction was cooled, solvent was evaporated and the residue was chromatographed (SiO₂; CH₂Cl₂:MeOH) to furnish 36 (1.7 g, 92%) as an orange oil that slowly crystallizes.

Synthesis of 37: To a solution of 36 (1.85 g, 5.8 mmol) in 95% EtOH (10 mL) was added Raney Ni (400 mg) and the mixture was shaken under an atmosphere of H₂ (30 psi) for 4 hours. Then the mixture was filtered through Celite and evaporated. The oil was chromatographed (SiO₂; CH₂Cl₂:MeOH:TEA) to furnish 37 (1.4 g, 83%) as a slightly brown oil. (¹H NMR, CDCl₃): 7.0 (d, 1H), 6.9 (t, 1H), 6.7 (t, 2H), 3.9 (bs, 2 NH), 3.7 (t, 4H), 2.9 (t, 4H), 2.7-2.4 (m, 1OH), 1.8 (bs, 2H).

Synthesis of RL1025, 4-(2-(4-[2-(4-t-butylcarboxamidobenzenesulfonamido)phenyl]-piperazin-1-yl)-ethyl)-morpholine: To a solution of 37 (200 mg, 0.69 mmol) and triethylamine (100 mg, 1.5 equiv) in CH₂Cl₂ (10 mL) was added the 16 (204 mg, 1 equiv). After stirring 12 hours, water was added and the organic phase was separated, dried (MgSO₄) and evaporated. The residue was chromatographed (SiO₂; CH₂Cl₂:MeOH:TEA) to furnish the title compound (300 mg, 80%) as a white solid. (¹H NMR, CDCl₃): 8.1 (bs, NH), 7.85 (d, 2H), 7.75 (d, 2H), 7.55 (d, 1H), 7.1-6.99 (m, 3H), 5.95 (s, NH), 3.75 (s, 4H), 2.6 (m, 16H), 1.45 (s, 9H).

Synthesis of RL1026, 4-(2-(4-[2-(4-phenylcarboxamidobenzenesulfonamido)phenyl]-piperazin-1-yl)-ethyl)-morpholine: Was made in similar fashion to RL1025 using 29. (¹H NMR, CDCl₃): 8.05 (s, NH), 7.95 (s, NH), 7.85 (m, 4H), 7.7 (d, 1H), 7.6 (t, 2H), 7.5 (t, 2H), 7.1 (m, 2H), 7.05 (t, 1H), 3.7 (t, 4H), 2.6 (m, 8H), 1.7 (s, 6H).

The synthesis of RL1017 is shown in Scheme 13. The secondary amine of 35 is protected by reaction with t-butyloxycarbonyl anhydride. The nitro group was then reduced by hydrogen and Raney Ni catalysis. The aniline, 39, was then reacted with 16 in the presence of triethylamine to form the sulfonamide 40. Finally, the target compound was furnished by deprotection with aqueous HCl in acetonitrile. Synthesis of 38: To a solution of 35 (850 mg, 4.1 mmol) and triethylamine (540 mg, 1.3 equiv) in CH₂Cl₂ (10 mL) was added di-t-butyl dicarbonate (985 mg, 1.1 equiv). Reaction was complete after stirring 8 hours. Water was added and the organic phase was separated, dried (MgSO₄) and evaporated. The residue was chromatographed (SiO₂; CH₂Cl₂:MeOH) to yield 38 (1.1 g, 87%) as an orange oil. (1H NMR, CDCl₃): 7.8 (d, 1H), 7.5 (dd, 1H), 7.1 (m, 2H), t, 4H), 3.6 (t, 4H), 3.0 (bs, 4H), 1.5 (s, 9H).

Synthesis of 39: To a solution 38 (1.25 g, 4.1 mmol) in 95% EtOH (50 mL) was added Raney Ni (400 mg) and the mixture was shaken under an atmosphere of H₂ (30 psi). After 6 hours, the mixture was filtered through Celite and evaporated. The oil was chromatographed (SiO₂; CH₂Cl₂:MeOH) to yield 39 (1 g, 88%) as a lilac solid. (¹H NMR, CDCl₃): 7.0 (d, 2H), 6.75 (d, 2H), 4.2 (q, 2H), 3.6 (bs, 6H), 2.9 (bs, 4H), 1.3 (t, 3H).

Synthesis of 40: To a solution of 39 (288 mg, 1.04 mmol) and triethylamine (126 mg, 1.2 equiv) in CH₂Cl₂ (5 mL) at 0° C. was added 16 (319 mg, 1 equiv) in one portion. After stirring 12 hours, water was added and the organic layer separated, dried (MgSO₄) and evaporated. The remaining solid was chromatographed (SiO₂; CH₂Cl₂:MeOH) to furnish 40 (400 mg, 70%) as a white solid. (¹H NMR, CDCl₃): 8.4 (s, 1H), 8.05 (d, 1H), 7.4 (s, 2H), 7.2-6.9 (m, 4H), 5.9 (s, 1H), 4.2 (q, 2H), 3.9 (s, 3H), 3.6 (bs, 4H), 2.7 (bs, 4H), 1.4 (s, 9H), 1.3 (t, 3H).

Synthesis of RL1017, 2-Methoxy-4-t-butylcarboxamido-N-(2-piperazin-1-yl-phenyl)benzenesulfonamide: To a solution of 40 (210 mg, 0.384 mmol) in MeCN (3 mL) was added conc. HCl (3 drops). After 10 hours, solvent was evaporated and the remaining solid was chromatographed (SiO₂; CH₂Cl₂:MeOH:TEA) to furnish the title compound (170 mg, 99%) as a tan solid. (¹H NMR, CDCl₃): 8.05 (d, NH), 7.4 (m, 2H), 7.2-7.1 (m, 3H), 6.95 (m, 2H), 5.95 (bs, NH), 3.95 (s, 3H), 3.15 (s, 2H), 2.85 (s, 2H), 1.7 (bs, 4H), 1.5 (s, 9H).

The synthesis of RL1024 is shown in Scheme 14. Reaction of 2-nitrobenzyl bromide (22) with 4-hydroxypiperidine affords alcohol 41. Reduction with hydrogen and Raney Ni affords aniline 42. Reaction with sulfonyl chloride 29 affords the target compound.

Synthesis of 42: To a solution of 22, 2-nitrobenzyl bromide (500 mg, 2.3 mmol), and triethylamine (349 mg, 1.5 equiv) in 95% EtOH (3 mL) was added 4-hydroxypiperidine (230 mg, 1 equiv) and the mixture was refluxed. After 1 hour, solvent was evaporated and the residue was partitioned between H₂O and CH₂Cl₂. The organic layer was separated, dried (MgSO₄) and chromatographed (SiO₂; CH₂Cl₂:MeOH) to furnish 41 (488 mg, 90%) as a colorless oil. This oil was then dissolved in 95% (20 mL) and Raney Ni (100 mg) was added and the mixture was shaken under an atmosphere of H₂ (30 psi). After 2 hours, the mixture was filtered through Celite and solvent was evaporated and the resulting solid was recrystallized (Hex:EtOAc:MeOH) to furnish 42 (400 mg, 95%) as a colorless crystal. (¹H NMR, CDCl₃): 7.1 (t, 1H), 6.95 (d, 1H), 6.65 (m, 2H), 4.8 (bs, 2 NH), 3.7 (m, 1H), 3.5 (s, 2H), 2.7 (m, 2H), 2.1 (t, 2H), 1.9 (d, 2H), 1.5 (m, 2H).

Synthesis of RL1024, 1-(2-(4-Benzoylaminobenzenesulfonamido)benzyl)-piperidin-4-ol: To a solution of 42 (100 mg, 0.49 mmol) and triethylamine (98 mg, 2 equiv) in CH₂Cl₂ (2 mL) was added 29 (140 mg, 1 equiv). After stirring 12 hours, water was added and the organic phase was separated, dried (MgSO₄) and evaporated. The residue was chromatographed (SiO₂; CH₂Cl₂:MeOH) to furnish the title compound (113 mg, 50%). (¹H NMR, CDCl₃): 8.1 (s, 1H), 7.9 (d, 2H), 7.75 (s, 4H), 7.4 (m, 3H), 7.1 (d, 1H), 6.99 (d, 2H), 3.8 (bs, 1H), 3.2 (s, 2H), 2.7 (s, 2H), 2.2 (s, 2H), 1.9 (s, 2H), 1.6 (m, 2H).

### Example 5

Example for Compounds of Type III.3

The synthesis of RL1030 is described in Scheme 15. The isonipecotic acid first was protected with chloroformate to give 43. The carboxylic acid was then reduced and reoxidized to form the aldehyde 45. This was then coupled with phenyl hydrazine 46 or 47 and cyclized in situ to form 50 or 51. The hydrazine derivatives could then be coupled with the sulfonyl chlorides 11 or 29 to give 52, 53 or 54. Lastly, these compounds could be deprotected to give the desired compounds.

Synthesis of 43, 1-N-benzyloxycarbonyl-4-carboxy-piperidine: Isonipecotic acid (1.0 eq) and potassium carbonate (2.34 eq) were dissolved in water (0.77M). The solution was cooled to 10° C. and benzyl chloroformate (1.3 eq) was added dropwise. The solution was aged for twenty-two hours and extracted from methylene chloride (3×). The combined organics were washed with brine, dried over sodium sulfate, and concentrated to a thick colorless oil. Recrystallization from ether yielded a white solid. (95%): ¹H NMR data reported in Tetrahedron, Vol. 53, No. 32, pp. 10983-10992, 1997.

Synthesis of 44, 4-Hydroxymethyl-piperidine-l-carboxylic acid benzyl ester: To a solution of acid (43, 1.0 eq) in tetrahydrofuran, THF, (0.05M) cooled to 0° C. was added 1M borane in THF (3.0 eq) dropwise. The reaction mixture was aged for one hour and then quenched with IN sodium hydroxide (3.2 eq). The THF was evaporated under reduced pressure and the resulting oil was diluted with water and extracted with methylene chloride (3×). The combined organics were washed with brine and dried over sodium sulfate. The solvent was evaporated under reduced pressure to yield the title compound (97% Yield). ¹H NMR (400 MHz, CDCl₃): δ 1.167 (br d, 2H, J=10.4Hz), δ 1.656 (m, 1H), δ 1.736 (d, 2H, J=13.6Hz), δ 2.784 (br s, 2H), δ 3.498 (t, 2H, J=5.6Hz), δ 4.218 (br s, 2H), δ 5.124 (s, 2H), δ 7.354 (m, 5H).

Synthesis of 45, 4-Formyl-piperidine-1-carboxylic acid benzyl ester (The aldehyde readily air oxidizes to the carboxylic acid. To minimize air oxidation, the aldehyde should be stored under nitrogen at less than 0° C.) To a solution of alcohol 44 (1.0 eq) in methylene chloride (0.25M) was added pyridinium chlorochromate, PCC, (2.2 eq) in two portions. The clear solution became orange and then brown after the addition of PCC. The reaction was aged for two hours. The reaction mixture was filtered through celite and rinsed with excess ethyl acetate. The solvent was evaporated under reduced pressure and the residue was redissolved in ethyl acetate and washed with brine. The layers were separated and the organics were dried over magnesium sulfate. Purification by column chromatography using 3:2 ethyl acetate/hexanes yielded the title compound (67% Yield). ¹H NMR (400 MHz, CDCl₃): δ 1.588 (br d, 2H, J=6.8Hz), δ 1.919 (br s, 2H), δ 2.441 (m, 1H), δ 3.027 (t, 2H, J=11.2Hz), δ 4.064 (br s, 2H), δ 5.130 (s, 2H), δ 7.2-7.6 (m, 5H), δ 9.665 (s, 1H).

Synthesis of 50: To a solution of aldehyde 45 (1.0 eq) in methylene chloride (0.2M) at 0° C. was added the phenylhydrazine 46 (1.1 eq). After thirty minutes trifluoroacetic acid, TFA, (3.3 eq) was added to the clear solution dropwise. Addition of TFA caused the solution to turn purple. The reaction was aged for fifteen hours at 35° C. during which time the color changed to dark green. The solution was cooled to 0° C. and methanol (1/30 volume of solvent) was added followed by sodium borohydride (1.5 eq) which caused the color to change to olive green. The reaction mixture was allowed to react for three hours then quenched with 6% ammonium hydroxide (⅓ volume of solvent) and extracted with methylene chloride (3×). The combined organics were washed with brine and dried over magnesium sulfate. Purification by column chromatography using 2:3 ethyl acetate/hexanes yielded the title compound 50 (72% Yield). ¹H NMR (400 MHz, CDCl₃): δ 1.766 (br m, 4H), δ 3.027 (br s, 2H), δ 3.484 (s, 2H), δ 3.808 (s, 1H), δ 4.185 (br s, 2H), δ 5.212 (s, 2H), δ 6.611 (d, 1H, J=7.6Hz), δ 6.774 (t, 1H, J=7.2Hz), δ 7.064 (m, 2H), δ 7.407 (m, 5H).

Synthesis of 51: The synthesis of 51 is similar to the procedure for the synthesis of 50, but 4-bromophenylhydrazine (47) was used. The hydrazine was released from its hydrochloride with sodium methoxide in methanol. The aldehyde was added to the hydrazine followed by the dropwise addition of trifluoroacetic acid to the red solution, which causes a color to change to brown and then to purple/blue and finally to green. After the addition of sodium borohydride, the reaction turned red/orange. ¹H NMR (400 MHz, CDCl₃): δ 1.728 (br s, 4H), δ 2.958 (br s, 2H), δ 3.482 (s, 2H), δ 3.800 (br s, 1H), δ 4.130 (br s, 2H), δ 5.161 (s, 2H), δ 6.499 (d, 1H, J=8.4Hz), δ 7.084 (d, 1H, J=2), δ 7.125 (dd, 1H, J=8.4Hz J=2Hz), δ 7.375 (m, 5H).

Synthesis of 52: To a solution of spiroindoline 50 (1 eq) in methylene chloride (0.2M) cooled to 0° C. was added pyridine (1.5 eq) followed by sulfonyl chloride (29). The reaction mixture went from a yellow/beige mixture to a peach colored mixture to a pink solution in twenty minutes. The pink color indicates that the reaction is complete. The reaction mixture was quenched with water (0.2M) and extracted (3×) with methylene chloride. The combined organics were washed with brine and dried over magnesium sulfate. The solvent was evaporated under reduced pressure. Purification by column chromatography using a solvent gradient 1:2 ethyl acetate/hexanes to 2:3 ethyl acetate/hexanes to 3:2 ethyl acetate/hexanes yielded the title compound 52 (90%): ¹H NMR (400 MHz, CDCl₃) d 1.260 (br s, 2H), δ 1.662-1.885 (td, 2H, J=13.2Hz, J=4.4Hz), δ 2.785 (br s, 2H), δ 3.758 (s, 2H), δ 4.092-4.062 (d, 2H), δ 5.111 (s, 2H), δ 6.695-7.034 (m, 2H), δ 7.194-7.335 (m, 6H), δ 7.422 (t, 2H, J=7.6Hz), δ 7.526 (t, 1H, J=8Hz), δ 7.632 (d, 1H, J=8Hz), δ 7.773-7.838 (m, 6H), δ 8.597 (br d, 1H, J=18.8Hz).

Synthesis of 53: The synthesis of 53 is similar to the procedure for the synthesis of 52, with spiroindoline 50 coupling to sulfonyl chloride 11. ¹H NMR (400 MHz, CDCl₃) δ 1.4 (s, 9H), δ 1.5 (m, 2H), δ 1.75 (m, 2H), δ 2.85 (m, 2H), δ 3.7 (s, 3H), δ 4.0 (s, 2H), δ 4.1 (m, 2H), δ 5.15 (s, 2H), δ 5.95 (s, 1H), δ 7.2 (m, 11H), δ 8.05 (d, 1H).

Synthesis of 54: The synthesis of 54 is similar to the procedure for synthesis of 52, but with spiroindoline 51 and sulfonyl chloride 11. The reaction mixture went from an orange/brown solution to pink. Purification by column chromatography yielded the sulfonamide (93%): ¹H NMR (300 MHz, CDCl₃) δ 1.469 (s, 9H), δ 1.714 (br m, 2H), δ 2.811 (br m, 2H), δ 3.703 (s, 3H), δ 3.965 (s, 2H), δ 4.157 (br s, 2H), δ 5.138 (s, 2H), δ 6.030 (br s, 1H), δ 7.1-7.5 (m, 10H), δ 8.012 (d, 1H, J=7.5Hz).

Synthesis of RL1030: To a solution of 52 (1 eq) in methylene chloride (0.2M) cooled to 0° C. was added trimethylsilyl iodide, TMS-I. The reaction mixture was warmed to room temperature and two hours later a second equivalent of TMS-I was added and the reaction was aged for 12 hours. The reaction mixture was quenched with 50% methanol/water (0.5M) and the organic solvent was evaporated under reduced pressure. Sodium carbonate was added to the aqueous layer. The reaction mixture was extracted with methylene chloride (3×) and the combined organics were washed with brine and dried over magnesium sulfate. Purification by column chromatography using 95:5:0.5 methylene chloride/methanol/triethylamine yielded the title compound (35%). ¹H NMR (400 MHz, CDCl₃): δ 1.278 (d, 2H, J=13.2Hz), δ 1.858 (m, 2H), δ 2.056 (m, 2H), δ 2.844 (d, 2H, J=10.4Hz), δ 3.567 (s, 2H), δ 3.745 (s, 2H), δ 6.98-7.851 (m, 18H), δ 8.175 (s, 1H).

Scheme 16 describes the synthesis of RL1029, RL1032 and RL1057.

Synthesis of RL1029 and RL1032: To a solution of 52 or 53 (1.0 eq) in methylene chloride (0.05M) was added methyl sulfide (27.0 eq) and boron trifluoride diethyl etherate (10.0 eq). The solution was stirred at room temperature for one and a half hours and then methyl sulfide (27.0 eq) was added again. The solution was allowed to stir for six hours. The solution was quenched with saturated sodium bicarbonate and stirred for one hour. The reaction mixture was extracted (3×) with chloroform. The combined organic layers were washed with brine and dried over sodium sulfate and the solvent was evaporated under reduced pressure to yield the title compound in quantitative amount which was purified using column chromatography.

RL1029 (¹H NMR, 300 MHz, CDCl₃): δ 1.286 (d, 2H, J=12.9Hz), δ 1.703 (td, 2H, J=13.0, J=4.2), δ 2.225 (br s, 1H), δ 2.652 (td, 2H, J=12.3Hz, 2.1Hz), δ 3.005 (d, 2H, J=12Hz), δ 3.793 (s, 2H), δ 7.016-7.077 (m, 2H), δ 7.213 (td, 1H, J=7.2Hz, J=1.8Hz), δ 7.453-7.850 (m, 10H), δ 8.126 (s, 1H).

RL1032 (¹H NMR, 300 MHz, CDCl₃): δ 1.456 (s, 9H), δ 1.821 (t, 2H, J=9.7Hz), δ 2.132 (br s, 1H), δ 2.679 (t, 2H, J=9.4Hz), δ 3.075 (d, 2H, J=9Hz), δ 3.474 (d, 2H, J=2.4Hz), δ 3.683 (s, 3H), δ 3.996 (s, 2H), δ 6.020 (s, 1H), δ 6.949-7.407 (m, 5H), δ 8.044 (d, 1H, J=6Hz).

Synthesis of 55 and 56: To a solution of deprotected spiroindoline RL1029 or RL1032 (1.0 eq) in methylene chloride (0.1M) cooled to −78° C. was added triethylamine. Chloroacetyl chloride was then added dropwise. The reaction mixture was stirred for forty-five minutes and then quenched with water and extracted (3×) with methylene chloride. The combined organics were washed with saturated sodium bicarbonate and then washed with brine and dried over sodium sulfate. The solvent was evaporated under reduced pressure to yield the title compound that was purified by column chromatography using a solvent gradient 3:2 ethyl acetate/hexanes_{—}4:1 ethyl acetate/hexanes (68%):

54 (¹H NMR, 400 MHz, CDCl₃): δ 1.326 (d, 1H, J=14.8), δ 1.468 (d, 1H, J=12.8Hz), δ 1.639-1.806 (m, 2H), δ 2.678 (t, 1H, J=11.6Hz), δ 3.186 (t, 1H, J=12.8Hz), δ 3.768-3.878 (m, 3H), δ 4.074 (dd, 2H, J=24Hz, J=12.4Hz), δ 4.480 (d, 1H, J=13.2Hz), δ 6.999-7.864 (m, 13H), δ 8.182 (s, 1H).

55 (¹H NMR, 400 MHz, CDCl₃): δ 1.45 (s, 9H), 8 1.80 (m, 4H), δ 2.65 (t, 1H), δ 3.15 (t, 1H), δ 3.7 (s, 3H), δ 3.85 (br d, 1H), δ 4.0 (s, 2H), δ 4.1 (m, 2H), δ 4.55 (br d, 1H), δ 6.05 (br s, 1H), δ 7.25 (m, 4H), δ 7.45 (s, 1H), δ 8.05 (d, 1H).

Synthesis of RL1031 and RL1054: To a solution of 55 (1.0 eq) in THF (0.1M) was added morpholine or N-methyl piperazine (2.2 eq). The reaction mixture was refluxed for three hours. The mixture was quenched with water and extracted with methylene chloride (4×). The combined organics were washed with brine and dried over sodium sulfate. Purification by column chromatography using 5% methanol in ethyl acetate yielded the title compounds:

RL1031 (¹H NMR, 400 MHz, CDCl₃): δ 1.293 (br m, 1H), δ 1.274 (br d, 1H, J=13.6Hz), δ 1.633 (m, 2H), δ 2.520 (m, 4H), δ 2.600 (t, 1H, J=12.8Hz), δ 3.072 (t, 2H, J=12.4), δ 3.195 (dd, 1H, J=41.2Hz, J=13.6Hz), δ 3.696 (m, 4H), δ 3.814 (dd, 2H, J=36Hz, J=10.8Hz), δ 4.006 (d, 1H, J=13.6Hz), δ 4.479 (d, 1H, J=12.8Hz), δ 6.957 (d, 1H, J=7.2Hz), δ 7.029 (t, 1H, J=7.6Hz), δ 7.474 (t, 1H, J=8Hz), δ 7.546-7.860 (m, 1OH), δ 8.350 (s, 1H).

RL1054 (¹H NMR, 400 MHz, CDCl₃) δ 1.269 (d, 1H, J=14Hz), δ 1.455 (d, 1H, J=12Hz), δ 1.592 (td, 1H, J=12.8Hz, J=4Hz), δ 1.699 (td, 1H, J=12.8Hz, J=4Hz), δ 2.286 (s, 3H), δ 2.530 (br m, 9H), δ 3.044 (d, 1H, J=12.4Hz), δ 3.171 (dd, 1H, J=51.2Hz , J=13.2Hz), δ 3.818 (dd, 2H, J=35.6Hz, J=10.4Hz), δ 4.041 (d, 1H, J=13.2Hz), δ 4.485 (d, 1H, J=13.6Hz), δ 6.967 (m, 1H), δ 7.035 (t, 1H, J=7.0), δ 7.224-7.862 (m, 11H), δ 8.275 (s, 1H).

Synthesis of RL1055: The synthesis of RL1055 is similar to the procedure for the synthesis of RL1031, but 56 was used instead of 55 to couple with morpholine. ¹H NMR (400 MHz, CDCl₃): δ 1.45 (s, 9H), δ 1.60 (m, 8H), δ 1.70 (m, 4H), δ 2.65 (t, 1H), δ 3.10 (t, 1H), δ 3.75 (s, 3H), δ 3.9 (m, 3H), δ 4.0 (s, 2H), δ 4.6 (d, 1H), δ 6.0 (s, 1H), δ 7.2 (m, 4H), δ 7.45 (s, 1H), δ 8.05 (d, 1H).

### Example 6

Example for Compounds of Type III.4

The approach towards the synthesis of RL1007- RL1014 is shown in Scheme 17 & 18. Coupling of the appropriate benzophenone (56 or 57) with N-Boc-L-alanine, isobutylchloro-formate and N-methyl morpholine yielded the desired product. The BOC protecting group was easily cleaved with HCl in EtOAc. Adjustment of the pH to 8 by the addition of NaOH and stirring for 14 h yielded the benzodiazepines 58 and 59. The entire sequence requires only one purification. For compounds where R is hydrogen, such as benzodiazepine 62, the same basic strategy was employed starting from ethylene glycol acetal of 2-amino- benzaldehyde (Scheme 18). Coupling of the 3 benzodiazepine (58, 59 or 62) with a benzyl chlorides h, i, j, k, l or m in the presence of NaH and DMF yielded products, RL1007- RL 1013. The benzyl chlorides h, i, j, k, l and m were synthesized easily from 4-chloromethylbenzoyl chloride and an appropriate amine at −78° C. Simple hydrolysis of RL1013 with LiOH yielded RL1014.

General procedure for the synthesis of 4-chloromethyl benzamides h, i, j, k, l and m: To a solution of 4-chloromethylbenzoyl chloride (1 eq) in dichloromethane at −78° C., triethylamine (1.2 eq) was added followed by a primary or secondary amine (1 eq). The reaction mixture was allowed to warm to −40° C. over 0.5 h and quenched with water (10 mL) and extracted with dichloromethane. The combined organic layers were washed with brine, dried over Na₂SO₄ and evaporated to give a crude solid. This crude solid was either crystallized from CH₂Cl₂ and hexanes or subjected to column chromatography in 5% MeOH in CH₂Cl₂ to give the requisite product in moderate to good yield.

h (NMR, CDCl₃): 7.7 (d, 2H, J=8.4 Hz), 7.4 (d, 2H, J=8.4 Hz), 5.9 (br s, 1H), 4.6 (s, 2H), 1.5 (s, 9H).

i (NMR, CDCl₃): 7.9 (m, 3H), 7.7 (d, 2H, J=7.8 Hz), 7.5 (d, 2H, J=7.8 Hz), 7.4 (m, 2H), 7.2 (m, 1H), 5.3 (s, 2H).

j (NMR, CDCl₃): 7.8 (m, 2H), 7.5 (m, 2H), 6.7 (br s, 1H), 4.6 (s, 2H), 4.3 (d, 2H, J=5.1 Hz), 3.8 (s, 3H).

k (NMR, CDCl₃): 7.2-7.5 (m, 9H), 4.6 (s, 2H), 3.8 (br s, 2H), 3.6 (s, 2H), 3.4 (br s, 2H), 2.6 (br s, 2H), 2.4 (br s, 2H).

l (NMR, CDCl₃): 8.5 (d, 1H, 8.1 Hz), 8.0 (br s, 1H), 7.6-7.2 (m, 12H) 4.6 (s, 2H).

m (NMR, CDCl₃): 10.61 (br s, 1H), 8.47 (d, 2H, J=4.8 Hz, 7.8 (d, 2H, J=8.4 Hz), 7.7 (d, 2H, J=4.8 Hz), 7.6 (d, 2H, J=8.4 Hz), 4.8 (s, 2H).

Synthesis of 58: A solution of N-t-BOC-L-alanine (1.5 eq) in THF was cooled to −15° C. and treated with N-Methylmorpholine (1.5 eq). Isobutyl chloroformate (1.5 eq) was then slowly added to the mixture. The solution was stirred for 5 minutes and then 2-aminobenzophenone (1 eq) was added. The mixture was allowed to warm to room temperature and stirred overnight. The solution was diluted with water and extracted into methylene chloride. The organic extract was washed with water, 0.1N NaOH, brine and dried over Na₂SO₄. The solvent was evaporated to give a thick oil. This oil was dissolved in ethyl acetate and cooled to 0° C. and conc. HCl was added to the solution and stirred for 24 h. The solvent was evaporated to give a solid that was dissolved in MeOH and the pH was adjusted to 8 by the addition of 1N NaOH and stirred overnight at room temperature. Most of the methanol was evaporated, water was added and extracted into methylene chloride. The extract was washed with brine and dried with Na₂SO₄. The solvent was evaporated and the residue purified in 1:3 ethylacetate:hexanes to give the requisite benzodiazepine 52 in 60% overall yield. (¹H NMR, CDCl₃): 9.2 (br s, 1H), 7.6-7.1 (m, 9H), 3.8 (q, 1H, J=6.6 Hz), 1.8 (d, 3H, J=6.6 Hz)

Synthesis of 59 was achieved using the procedure described for compound 58 using 2-amino acetophenone, 57. (¹H NMR, CDCl₃): 9.5 (br s, 1H), 7.6 (d, 1H, J=7.8 Hz), 7.5 (m, 2H), 7.2 (m, 2H), 3.6 (q, 1H, J=6.6 Hz), 2.4 (s, 3H), 1.6 (d, 3H, J=6.6 Hz).

Synthesis of 60: A mixture of 2-nitrobenzaldehyde (1 eq), ethylene glycol (2 eq) and p-toluenesulfonic acid (catalyst) in toluene was refluxed for 15 h using a Dean-Stark trap. The toluene solution was washed twice with water, dried with Na₂SO₄ and evaporated in vacuo to give 60, a brown oil. (¹H NMR, CDCl₃): 7.9 (d, 1H, J=8.1 Hz), 7.8 (d, 1H, J=8.1 Hz), 7.6 (m, 1H), 7.5 (m, 1H), 6.5 (s, 1H), 4.0 (m, 4H).

Synthesis of 61: This crude oil (60) was dissolved in EtOH and subjected to catalytic hydrogenation at 40 psi using Raney Ni as the catalyst. The mixture was filtered through a pad of celite cautiously and washed with copious amounts of MeOH. The solvent was evaporated under reduced pressure and purified by column chromatography quickly to yield the desired product. (¹H NMR, CDCl₃): 4.3-4.0 (m, 6H), 5.92 (s, 1H), 7.4 (d, 1H, J=7.8 Hz), 7.2 (m, 1H), 6.8 (m, 1H), 6.7 (d, 1H, J=7.8 Hz).

Synthesis of 63 was achieved using the procedure described for compound 58 using compound 62. (¹H NMR, CDCl₃): 9.0 (br s, 1H), 8.6 (d, 1H, J=2.4 Hz), 7.5 (m, 2H), 7.3 (m, 1H), 7.1 (d, 1H, 8.1 Hz), 3.7 (qd, 1H, J=2,4 Hz, 6.6 Hz), 1.7 (d, 3H, J=6.6 Hz).

General procedure for the synthesis of RL1007-RL1013: To a solution of NaH (2 eq) in DMF under nitrogen, benzodiazepine 58, 59 or 62 (1 eq) was added and stirred for 30 min. Then a solution of 4-chloromethyl benzamide h, i, j, k, l or m (1.2 eq) in CH₂Cl₂ was added slowly at room temperature and the mixture stirred for 14 h. The solution was diluted with 10 mL of water and extracted into dichloromethane. The organic extract was washed with water, brine and dried over Na₂SO₄. Flash column chromatography was used to purify the products.

RL1007 (NMR, CDCl₃): 7.8-7.1 (m, 13H), 6.9 (br s, 1H), 5.4 (d, 1H, J=15.6 Hz), 4.9 (d, 1H, J=15.6 Hz), 3.7 (q, 1H, J=6.6 Hz), 2.4 (s, 3H), 1.7 (d, 3H, J=6.6 Hz).

RL1008 (NMR, CDCl₃): 7.8-7.1 (m, 18 H), 6.9 (br s, 1H), 5.8 (d, 1H, J=15.3 Hz), 4.9 (d, 1H, J=15.3 Hz), 3.9 (q, 1H, J=6.6 Hz), 1.8 (d, 3H, J=6.6 Hz).

RL1009 (NMR, CDCl₃): 7.6 (d, 2H, J=8.1 Hz), 7.5 (m, 1H), 7.4 (m, 1H), 7.2 (m, 2H), 7.1 (m, 2H), 5.9 (br s, 1H), 5.4 (d, 1H, J=15.6 Hz), 4.9 (d, 1H, J=15.6 Hz), 3.7 (q, 1H, J=6.6 Hz), 2.4 (s, 3H), 1.6 (d, 3H, J=6.6 Hz), 1.4 (s, 9H).

RL1010 (NMR, CDCl₃): 7.5-7.1 (m, 13H), 5.3 (d, 1H, J=15.9 Hz), 5.0 (d, 1H, J=15.9 Hz), 3.8 (br m, 3H), 3.7 (s, 2H), 3.4 (br s, 2H), 2.5 (br s, 2H), 2.4 (br m, 5H), 1.7 (d, 3H, J=6.6 Hz).

RL1011 (NMR, CDCl₃): 7.5-7.1 (m, 18 H), 5.7 (d, 1H, J=15.6 Hz), 4.9 (d, 1H, J=15.6 Hz), 3.9 (q, 1H, J=6.6 Hz), 3.8 (br s, 2H), 3.5 (br s, 2H), 3.3 (br s, 2H), 2.5 (br s, 2H), 2.3 (br s, 2H), 1.8 (d, 3H, J=6.6 Hz).

RL1012 (NMR, CDCl₃): 8.6 (d, 1H, J=2.7 Hz), 7.5-7.1 (m, 13H), 5.2 (m, 2H), 3.8 (br m, 5H), 3.6 (s, 2H), 3.5 (br s, 2H), 2.5 (br s, 2H), 2.4 (br s, 2H), 1.8 (d, 3H, J=6.6 Hz).

RL1013 (NMR, CDCl₃): 8.6 (d, 1H, J=2.7 Hz), 7.7 (d, 2H, J=8.4 Hz), 7.5 (m, 2H), 7.2 (m, 4H), 6.6 (br s, 1H), 5.3 (d, 1H, J=16.2 Hz), 5.2 (d, 1 H, J=16.2 Hz), 4.2 (d, 2H, J=5.1 Hz), 3.8 (m, 4H), 1.8 (d, 3H, J=6.6 Hz).

Synthesis of RL1014: To a solution of RL1013 in methanol: water:3:1, LiOH (1.5 eq) was added and stirred for 15 h. The solvent was removed under vacuum, 1N HCl was added and the pH adjusted to 3 and then extracted into ethyl acetate. The combined organic layers were washed with brine, and dried over Na₂SO₄. Recrystallization from dichloromethane/hexanes yielded RL1014. (NMR, CDCl₃): 8.7 (d, 1H, J=2.4 Hz), 7.6 (d, 2H, J=8.1 Hz), 7.5 (m, 1H), 7.4 (m, 2H), 7.3 (m, 1H), 7.1 (m, 2H), 6.9 (br m, 1H), 5.6 (d, 1H, J=15.9 Hz), 4.9 (d, 1H, J=15.9 Hz), 4.2 (m, 2H), 3.8 (m, 1H), 1.8 (d, 3H, J=6.6 Hz).

The approach towards the synthesis of RL1046- RL1050 is shown in Scheme 19. Coupling of L-prolinemethylester hydrochloride with 2-nitrobenzylbromide yielded the desired compound 64 in good yield. The reduction of nitro group was achieved using 40 psi of H₂ in the presence of catalytic Raney Ni. The formation of the seven membered ring proceeded smoothly in 3N HCl under refluxing conditions. Coupling of the benzodiazepine with benzyl chlorides h, i, l, m and n in the presence of NaH and DMF yielded the desired products RL1046-RL1050. Benzyl bromide n was synthesized from 4-aminobenzylalcohol. Thus, treatment of 4-aminobenzylalcohol with pivaloyl chloride gave the desired compound in good yield. Bromination of the alcohol was achieved using CBr₄ and PPh₃ to yield the desired product.

Synthesis of n: To a solution of 4-aminobenzyl alcohol (1 eq) in dichloromethane, triethylamine (1.1 eq) was added at −78° C. followed by pivaloyl chloride. After stirring for 0.5 h at this temperature the reaction mixture was poured into ice/water mixture and extracted with dichloromethane. The combined organic layers were washed with brine, dried over Na₂SO₄. Recrystallization from dichloromethane/hexanes yielded the amide (NMR, CDCl₃): 7.5 (d, 1H, J=8.4 Hz), 7.3 (d, 1H, J=8.4 Hz), 4.6 (s, 2H), 1.3 (s, 9H). To a mixture of the amide in dichloromethane, PPh₃ (1.5 eq) was added followed by CBr₄ (1.5 eq). After stirring the reaction at room temperature for 2 h, water was added and extracted with dichloromethane. The combined organic layers were washed with brine, dried over Na₂SO₄. Flash column chromatography with 1:4 ethyl acetate:hexanes yielded n.

n (NMR, CDCl₃): 7.5 (d, 2H, J 8.7 Hz), 7.3 (d, 2H, J=8.7 Hz), 4.5 (s, 2H), 1.3 (s, 9H).

Synthesis of 64: To a solution of prolinemethylester hydrochloride (1 eq) in dichloromethane was added triethylamine (2.5 eq) at 0° C. followed by a solution of 2-nitrobenzylbromide (1 eq) in dichloromethane slowly. The solution was stirred for 15 h at room temperature. The reaction mixture was diluted with water and extracted with methylene chloride. The combined organics were washed with brine, dried over Na₂SO₄ and the solvent was removed under reduced pressure. Chromatography using 1:4 Ethylacetate:hexanes gave the title compound in good yield. (NMR, CDCl₃): 7.9 (d, 1H, J=8 Hz), 7.8 (d, 1H, J=8 Hz), 7.6 (m, 1H), 7.4 (m, 1H), 4.15 (m, 2H), 3.65 (s, 3H), 3.4 (m, 1H), 3.1 (m, 1H), 2.5 (m, 1H), 2.25 (m, 1H), 2.2-1.8 (m, 3H).

Synthesis of 65 and 66: Compound 64 was dissolved in EtOH and hydrogenated at 40 psi using catalytic Raney Nickel. The crude amine 65 obtained after filtration through Celite was refluxed with 3N HCl for 5 h. The reaction mixture was then cooled to room temperature and the pH of the medium was adjusted to 10 with 1N NaOH and extracted with methylene chloride. The combined organics were washed with brine, dried over Na₂SO₄ and the solvent was removed under reduced pressure. Purification of the crude material using 3:2 Ethyl acetate : Hexanes yielded the desired compound 66 in moderate yield.

65 (NMR, CDCl₃): 7.6-7.4 (m, 2H), 6.65 (m, 2H), 4.8 (br s, 2H), 4.0 (d, 1H, J=11 Hz), 3.65 (s, 3H), 3.3 (d, 1H, J=11 Hz), 3.2 (m, 1H), 2.9 (m, 1H), 2.3-2.1 (m, 2H), 2.0-1.8 (m, 3H).

66 (NMR, CDCl₃) 7.8 (br s, 1H), 7.4-7.3 (m, 2H), 7.2 (m, 1H), 7.0 (m, 1H), 4.0 (d, 1H, J=11.7 Hz), 3.7 (m, 2H), 3.1 (m, 1H), 2.7-2.4 (m, 2H), 2.1-1.8 (m, 3H).

General procedure for the synthesis of RL1046-RL1050: To a suspension of NaH (2 eq) in DMF at room temperature was added the diazepine 66 (1 eq). After stirring for 30 minutes at room temperature, a solution of the benzyl halides in dichloromethane was added slowly and the reaction mixture was stirred for 18 h, after which it was poured into ice water. Extraction with dichloromethane followed by washing the combined organic layers with brine and drying with Na₂SO₄ gave the desired product in crude form. Flash column chromatography using 3:2 Ethyl acetate: Hexanes yielded the title compounds in moderate yield.

RL1046 (NMR, CDCl₃): 7.6 (d, 2H, J=8.4Hz), 7.4-7.2 (m, 6H), 5.9 (br s, 1H), 5.3 (d, 1H, 15 Hz), 4.9 (d, 1H, J=15Hz), 3.7 (m, 2H), 3.2 (d, 1H, J=11.1 Hz), 3.1 (m, 1H), 2.5 (m, 2H), 2.1-1.8 (m, 3H), 1.5 (s, 9H).

RL1047 (NMR, CDCl₃): 8.0 (br s, 1H), 7.8 (d, 2H, J=8.1 Hz), 7.6 (d, 2H, J=7.5 Hz), 7.4 (m, 7H), 7.2 (d, 1H, J=7.5Hz), 7.1 (m, 1H), 5.3 (d, 1H, J=15 Hz), 5.0 (d, 1H, J=15 Hz), 3.8 (d, 1H, J=11.4 Hz), 3.7 (m, 1H), 3.2 (d, 1H, J=11.4 Hz), 3.1 (m, 1H), 2.5 (m, 2H), 2.1-1.8 (m, 3H).

RL1048 (NMR, CDCl₃): 8.5 (d, 1H, J=8.7Hz), 8.0 (br s, 1H), 7.6-7.2(m, 16H), 5.2 (d, 1H, J=15 Hz), 5.0 (d, 1H, J=15 Hz), 3.8 (d, 1H, J=11.1 Hz), 3.1 (m, 1H), 2.5 (m, 2H), 2.1-1.8 (m, 3H).

RL1049 (NMR, CDCl₃): 8.5 (m, 2H), 8.0(br s, 1H), 7.8 (d, 2H, J=8.1 Hz), 7.6 (m, 2H), 7.4 (m, 4H), 7.2 (m, 2H), 5.3 (d, 1H, J=15 Hz), 5.0 (d, 1H, J=15 Hz), 3.8 (d, 1H, J=10.8 Hz), 3.7 (m, 1H), 3.2 (d, 1H, J=10.8 Hz), 3.1 (m, 1H), 2.5 (m, 2H), 2.1-1.8 (m, 3H).

RL1050 (NMR, CDCl₃): 7.4 (d, 2H, J=8.4 Hz), 7.3-7.1 (m, 6H), 5.3 (d, 1H, J=14.4 Hz), 4.8 (d, 1H, J=14.4 Hz), 3.7 (d, 1H, J=11.1 Hz), 3.6 (m, 1H), 3.2 (d, 1H, J=11.1 Hz), 3.1 (m, 1H), 2.5 (m, 2H), 2.1-1.8 (m, 3H), 1.3 (s, 9H).

The synthesis of RL1021 and RL1022 are shown in Scheme 20. In much the same way as the cyclization to form the fused 7,5 ring system, the fused 7,6 rings were generated. Addition of racemic methyl pipecolinate to 2-nitrobenzyl bromide (22) in the presence of base formed the tertiary amine 67. Reduction of the nitro group with hydrogen and Raney Ni afforded the aniline 68. Then, intramolecular cyclization was effected by warm aqueous HCl to form the racemic diazepine 69. Deprotonation with hydride then reaction with benzyl chlorides i or m furnished the RL1021 and RL1022. The synthesis of RL1023 arrived from the reaction of deprotonated diazepine 69 with benzyl bromide o to afforded nitrobenzene compound 70. Reduction with hydrogen and Raney Ni followed by reaction with benzenesulfonyl chloride provided the target compound.

Synthesis of 67 and 68: To a solution of 2-nitrobenzyl bromide 22 (3.86 g, 1 eq) and triethylamine (3.6 g, 2 eq) in CH₂Cl₂ (50 mL) was added methyl pipecolinate hydrochloride (2.95 g, 17.9 mmol) and the mixture was stirred overnight. Water was added and the organic phase was separated, evaporated and chromatographed (SiO₂; CH₂Cl₂:MeOH) to obtain 67 (4.1 g, 82%) as a yellow oil. This oil was then dissolved in MeOH (50 mL) and Raney Ni (500 mg) was added and the mixture was shaken under an atmosphere of H₂ (30 psi) for 4 hours. The mixture was filtered through Celite and evaporated. The resulting oil was chromatographed (SiO₂; CH₂Cl₂:MeOH) to provide 68 (3.42 g, 77%) as a yellow oil. HMR (CDCl₃) 7.1 (t, 1H), 6.9 (d, 1H), 6.6 (d, 2H), 4.7 (bs, NH₂), 3.8 (d, 1H), 3.7 (s, 3H), 3.1 (d, 1H), 3.0-2.9 (m, 2H), 2.1-1.3 (m, 7H). Synthesis of 69: The oily 68 was suspended in 3M HCl (43 mL) and heated with stirring for 1 hour. The mixture was cooled, neutralized with sodium carbonate (13.6 g) and extracted with CH₂Cl₂. The extracts were collected, dried (MgSO₄) and evaporated to yield 69 (2.61 g, 87%) as a white solid. HMR (CDCl₃) 7.7 (bs, NH), 7.3-7.0 (m, 4H), 4.1 (d, 1H), 3.6 (d, 1H), 2.9 (bs, 1H), 2.8 (m, 1H), 2.6 (t, 1H), 1.9-1.6 (m, 5H), 1.3 (m, 1H).

Synthesis of RL1021, racemic 1-(4-Phenylcarboxamidobenzyl)-5,7,8,9,10,12-hexahydro-6aH-benzo[e]pyrido-[1,2-α][1,4]diazapin-6-one: To a solution of 69 (100 mg, 0.46 mmol) in DMF (2 mL) was added sodium hydride (13 mg, 1.2 equiv) and the mixture was stirred for 0.5 hour. Then i was added and the mixture was stirred overnight. Water was added and the mixture was extracted with CH₂Cl₂. The extracts were collected, dried (MgSO₄) and evaporated to an oil that was chromatographed (SiO₂; CH₂Cl₂:MeOH) to yield the title compound (65 mg, 33%) as a tan solid. HMR (CDCl₃): 7.85 (s, NH), 7.75 (d, 2H), 7.65 (d, 2H), 7.4-7.1 (m, 9H), 5.35 (d, 1H), 4.9 (d, 1H), 3.8 (d, 1H), 3.4 (bs, 1H), 2.8 (m, 2H), 2.5 (bs, 1H), 2.0-1.5 (m, 4H), 1.2 (m, 2H).

Synthesis of RL1022, racemic 1-(4-(o-Biphenyl)carboxamidobenzyl)-5,7,8,9,10,12-hexahydro-6aH-benzo[e]pyrido[1,2-α][1,4]diazapin-6-one: Was synthesized in similar fashion to RL1021 using m. HMR (CDCl₃): 8.5 (d, 1H), 7.9 (s, 1H), 7.6-7.2 (m, 17H), 5.3 (d, 1H), 4.9 (d, 1H), 3.8 (d, 1H), 3.4 (d, 1H), 2.7 (m, 2H), 2.5 (m, 1H), 1.9 (m, 2H), 1.6 (m, 4H).

Synthesis of 70: To a mixture of sodium hydride (44 mg, 1.3 equiv) in DMF (4 mL) was added 6-7-6 (300 mg, 1.39 mmol) in one portion. After stirring 30 minutes, 2-methoxy-5-nitrobenzyl bromide was added and the mixture was stirred overnight. The DMF was evaporated under reduced pressure and the remaining residue was partitioned between H₂O and CH₂Cl₂. The organic phase was separated, dried (MgSO₄) and evaporated to provide 70 (470 mg, 90%) as a solid, nearly pure by TLC (CH₂Cl₂:MeOH). HMR (CDCl₃): 8.15 (d, 1H), 8.1 (s, 1H), 7.4-7.15 (m, 4H), 6.9 (d, 1H), 5.1 (s, 2H), 4.0 (bs, 1H), 3.8 (s, 3H), 3.5 (bs, 1H), 2.8 (s, 2H), 2.5 (s, 1H), 2.0-1.2 (m, 6H).

Synthesis of 71: To a solution of 70 (470 mg, 1.23 mmol) in 95% EtOH (5 mL) was added Raney Ni (100 mg) and the mixture was shaken under an atmosphere of H₂ (30 psi) for 3 hours. The mixture was then filtered through Celite and evaporated. The solid was chromatographed (SiO₂; CH₂Cl₂:MeOH) to yield 71 (450 mg, 95%) as a white solid.

Synthesis of RL1023, racemic 1-(2-Methoxy-5-phenylsulfonylaminobenzyl)-5,7,8,9,10,12-hexahydro-6aH-benzo[e]pyrido[1,2-α] [1,4]diazapin-6-one: To a solution of 71 (50 mg, 0.13 mmol) and triethylamine (16 mg, 1.2 equiv) in CH₂Cl₂ (3 mL) was added in one portion benzenesulfonyl chloride (23 mg, 1 equiv). After 12 hours, water was added and the organic phase was separated, dried (MgSO₄) and evaporated. The resulting residue was chromatographed (SiO₂; CH₂Cl₂:MeOH) to obtain the title compound (40 mg, 59%) as a white solid. HMR (CDCl₃): 7.6 (d, 2H), 7.5 (m, 1H), 7.39-7.21 (m, 2H), 7.1 (d, 1H), 6.9 (d, 2H), 6.7 (bs, 1H), 6.6 (d, 2H), 5.0 (q, 2H), 3.9 (d, 1H), 3.6 (s, 3H), 3.4 (d, 1H), 2.85 (m, 2H), 2.7 (m, 2H), 2.6 (m, 2H), 2.5 (bt, 1H), 1.2 (bs 1H).

All publications, patents and patent documents are incorporated by reference herein, as though individually incorporated by reference. The invention has been described with reference to various specific and preferred embodiments and techniques. However, it should be understood that many variations and modifications will be obvious to those skilled in the art from the foregoing detailed description of the invention and may be made while remaining within the spirit and scope of the invention.

We claim:

1. A compound of the formula (I): 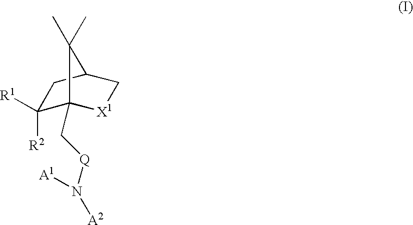
or its pharmaceutically acceptable salt thereof, wherein
X¹ is C(═Z¹) or CH₂;
Q is CH₂, C(═Z²), S, S(═Z³), (Z³═)S(═Z⁴), PA³, PA³(═O), P(═O)₂;
Z¹ and Z² are independently O, S or NA⁴;
Z³ and Z⁴ are independently O or NA⁵ wherein Z³ and Z⁴ both cannot be NA⁵;
A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,
A¹ and A² individually can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate;
R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹ and R² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

2. A compound of the formula (II): 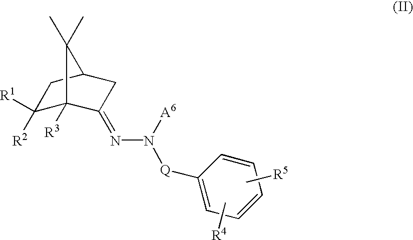
or its pharmaceutically acceptable salt thereof, wherein Q, R¹ and R² are defined above;
A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R⁴ and R¹ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

3. A compound of the formula (III.1): 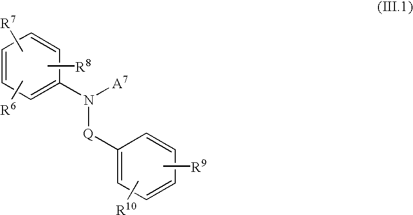
or its pharmaceutically acceptable salt thereof, wherein Q is defined above; and
A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
wherein if A⁷ and R^{6/8} independently come together to form a seven-membered bridged compound, then Q cannot be C(═O).

4. A compound of the formula (III.2): 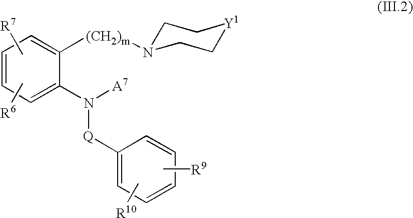
or its pharmaceutically acceptable salt thereof, wherein Q, A⁷, R⁶, R⁷, R⁹ and R¹⁰ are defined above;
m is 0 or 1;
Y⁸ is O, S, NA⁸ or CR¹¹R¹²; and
A⁸ is hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹¹ and R¹² independently can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

5. A compound of the formula (III.3): 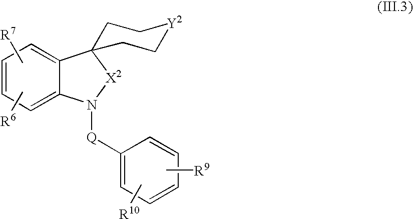
or its pharmaceutically acceptable salt thereof, wherein Q, R⁶, R⁷, R⁹ and R¹⁰ are defined above;
Y² is O, S, NA⁹ or CR¹⁵R¹⁶;
X² is C(═Z⁵) or CR¹⁷R¹⁸;
Z⁵ is O, S or NA¹⁰;
A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
R¹⁵ or R¹⁶ independently cannot be the following moiety:

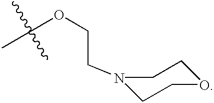

6. A compound of the formula (III.4): 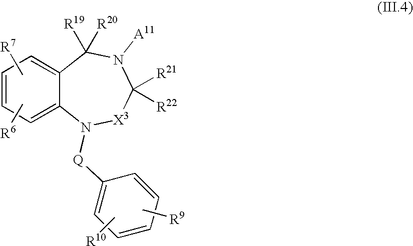
or its pharmaceutically acceptable salt thereof, wherein Q, R⁶, R⁷, R⁹ and R¹⁰ are defined above;
Y³ is O, S or NA¹¹;
X³ is C(═Z⁶);
Z⁶ is O, S or NA¹²;
A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate.

7. A pharmaceutical composition for the treatment or prophylaxis of a disorder mediated by a vasopressin receptor comprising an agonistic or antagonistic effective amount of a compound of the formula (I): 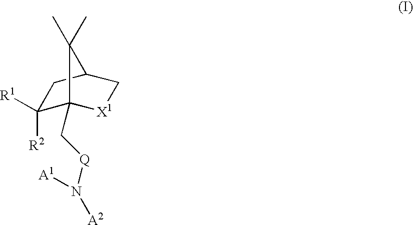
or its pharmaceutically acceptable salt there of, wherein
X¹ is C(═Z¹) or CH₂;
Q is CH₂, C(═Z²), S, S(═Z³), (Z³═)S(═Z⁴), PA³, PA³(═O) or p(═O)₂;
Z¹ and Z² are independently O, S or NA⁴;
Z³ and Z⁴ are independently O or NA⁵ wherein Z³ and Z⁴ both cannot be NA⁵;
A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,
A¹ and A² individually can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate;
R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R¹ and R² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in a pharmaceutically acceptable carrier or diluent.

8. A pharmaceutical composition for the treatment or prophylaxis of a disorder mediated by a vasopressin receptor comprising an agonistic or antagonistic effective amount of a compound of the formula (II): 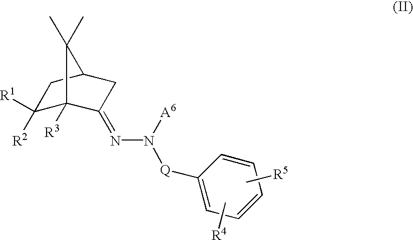
or its pharmaceutically acceptable salt thereof, wherein Q, R¹ and R² are defined above;
A¹ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R⁴and R⁵ as well as R^{4/5}and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in a pharmaceutically acceptable carrier or diluent.

9. A pharmaceutical composition for the treatment or prophylaxis of a disorder mediated by a vasopressin receptor comprising an agonistic or antagonistic effective amount of a compound of the formula (III.1): 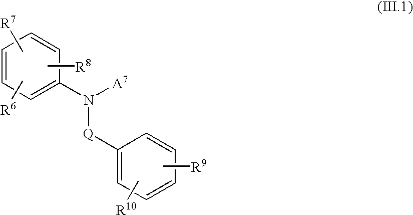
or its pharmaceutically acceptable salt thereof, wherein Q is defined above; and
A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
wherein if A⁷ and R^{6/8} independently come together to form a seven-membered bridged compound, then Q cannot be C(═O);
in a pharmaceutically acceptable carrier or diluent.

10. A pharmaceutical composition for the treatment or prophylaxis of a disorder mediated by a vasopressin receptor comprising an agonistic or antagonistic effective amount of a compound of the formula (III.2): 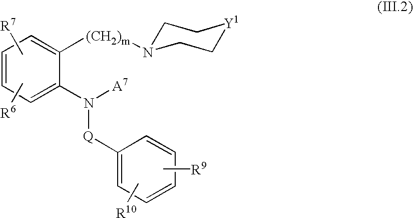
or its pharmaceutically acceptable salt thereof, wherein Q, A⁷, R⁶, R⁷, R⁹ and R¹⁰ are defined above;
m is 0 or 1;
Y¹ is O, S, NA⁸ or CR¹¹R¹² and
A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹¹ and R¹² independently can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in a pharmaceutically acceptable carrier or diluent.

11. A pharmaceutical composition for the treatment or prophylaxis of a disorder mediated by a vasopressin receptor comprising an agonistic or antagonistic effective amount of a compound of the formula (III.3): 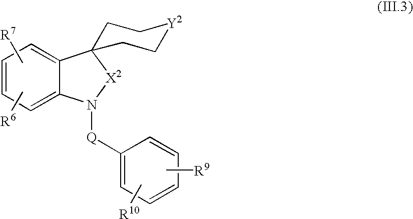
or its pharmaceutically acceptable salt thereof, wherein Q, R⁶, R⁷, R⁹ and R¹⁰ are defined above;
Y² is O, S, NA⁹ or CR¹⁵R¹⁶;
X² is C(═Z⁵) or CR¹⁷R¹⁸;
Z⁵ is O, S or NA¹⁰;
A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic or alkcarbonyl; and
R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
R¹⁵ or R¹⁶ independently cannot be the following moiety: 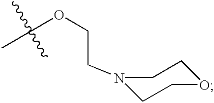
in a pharmaceutically acceptable carrier or diluent.

12. A pharmaceutical composition for the treatment or prophylaxis of a disorder mediated by a vasopressin receptor comprising an agonistic or antagonistic effective amount of a compound of the formula (III.4): 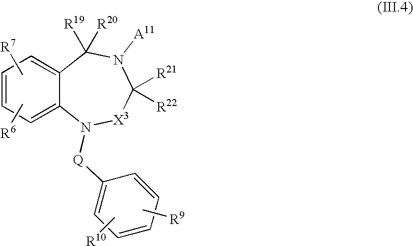
or its pharmaceutically acceptable salt thereof, wherein Q, R⁶, R⁷, R⁹ and R¹⁰ are defined above;
Y³ is O, S or NA¹¹;
X³ is C(═Z⁶);
Z⁶ is O, S or NA¹²;
A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in a pharmaceutically acceptable carrier or diluent.

13. A pharmaceutical composition for the treatment or prophylaxis of a disorder mediated by a vasopressin receptor comprising an agonistic or antagonistic effective amount of a compound of the formula (I): 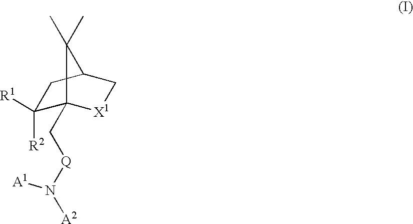
or its pharmaceutically acceptable salt there of, wherein
X¹ is C(═Z¹)or CH₂;
Q is CH₂, C(═Z²), S, S(═Z³), (Z³═)S(═Z⁴), PA³, PA³(═O) or p(═O)₂;
Z¹ and Z² are independently O, S or NA⁴;
Z³ and Z⁴ are independently O or NA⁵ wherein Z³ and Z⁴ both cannot be NA⁵;
A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,
A¹ and A² individually can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate;
R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R¹ and R² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination with one or more other effective vasopressin receptor agonists or antagonists, optionally in a pharmaceutically acceptable carrier or diluent.

14. A pharmaceutical composition for the treatment or prophylaxis of a disorder mediated by a vasopressin receptor comprising an agonistic or antagonistic effective amount of a compound of the formula (II): 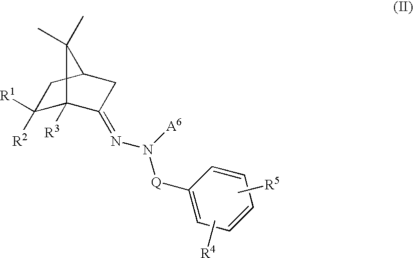
or its pharmaceutically acceptable salt thereof, wherein Q, R¹ and R² are defined above;
A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R⁴ and R⁵ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination with one or more other effective vasopressin receptor agonists or antagonists, optionally in a pharmaceutically acceptable carrier or diluent.

15. A pharmaceutical composition for the treatment or prophylaxis of a disorder mediated by a vasopressin receptor comprising an agonistic or antagonistic effective amount of a compound of the formula (III.1): 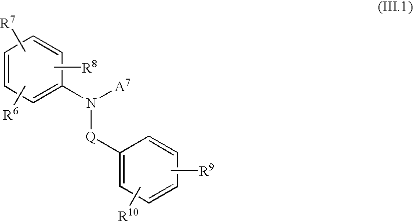
or its pharmaceutically acceptable salt thereof, wherein Q is defined above; and
A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
wherein if A¹ and R^{6/8} independently come together to form a seven-membered bridged compound, then Q cannot be C(═O);
in combination with one or more other effective vasopressin receptor agonists or antagonists, optionally in a pharmaceutically acceptable carrier or diluent.

16. A pharmaceutical composition for the treatment or prophylaxis of a disorder mediated by a vasopressin receptor comprising an agonistic or antagonistic effective amount of a compound of the formula (III.2): 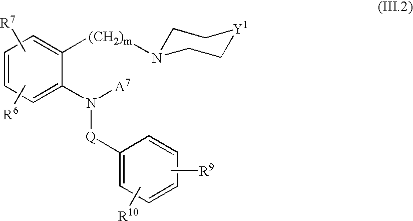
or its pharmaceutically acceptable salt thereof, wherein Q, A⁷, R⁶, R⁷ R⁹ and R¹⁰ are defined above;
m is 0 or 1;
Y¹ is O, S, NA⁸ or CR¹¹R¹²; and
A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹¹ and R¹² independently can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination with one or more other effective vasopressin receptor agonists or antagonists, optionally in a pharmaceutically acceptable carrier or diluent.

17. A pharmaceutical composition for the treatment or prophylaxis of a disorder mediated by a vasopressin receptor comprising an agonistic or antagonistic effective amount of a compound of the formula (III.3): 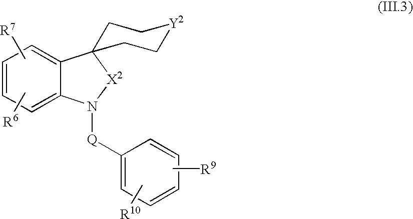
or its pharmaceutically acceptable salt thereof, wherein Q, R⁶, R⁷, R⁹ and R¹⁰ are defined above;
Y²is O, S, NA⁹or CR¹⁵R¹⁶;
X² is C(═Z⁵) or CR¹⁷R¹⁸;
Z⁵ is O, S or NA¹⁰;
A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic or alkcarbonyl; and
R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
R¹⁵ or R¹⁶ independently cannot be the following moiety: 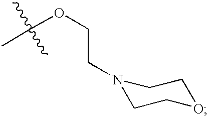
in combination with one or more other effective vasopressin receptor agonists or antagonists, optionally in a pharmaceutically acceptable carrier or diluent.

18. A pharmaceutical composition for the treatment or prophylaxis of a disorder mediated by a vasopressin receptor comprising an agonistic or antagonistic effective amount of a compound of the formula (III.4): 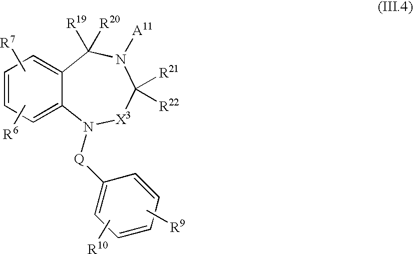
or its pharmaceutically acceptable salt thereof, wherein Q, R⁶, R⁷, R⁹ and R¹⁰ are defined above;
Y³ is O, S or NA¹¹;
X³ is C(═Z⁶);
Z⁶ is O, S or NA¹²;
A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination with one or more other effective vasopressin receptor agonists or antagonists, optionally in a pharmaceutically acceptable carrier or diluent.

19. A method for the treatment or prophylaxis of a disorder mediated by the vasopressin receptor comprising administering an agonistic or antagonistic effective amount of a compound of the formula (I): 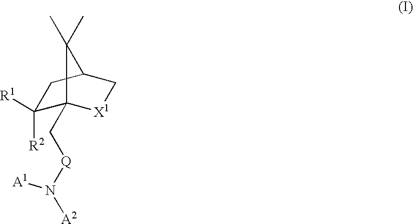
or its pharmaceutically acceptable salt thereof, wherein
X¹ is C(═Z¹) or CH₂;
Q is CH₂, C(═Z²), S, S(═Z³), (Z³═)S(═Z⁴), PA³, PA³(═O) or P(═O)₂;
Z¹ and Z² are independently O, S or NA⁴;
Z³ and Z⁴ are independently O or NA⁵ wherein Z³ and Z⁴ both cannot be NA⁵;
A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either A¹ or A² is an aromatic ring, preferably substituted with at least one carbonyl moiety; alternatively,
A¹ and A² individually can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate;
R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R¹ and R² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
optionally in a pharmaceutically acceptable carrier or diluent.

20. A method for the treatment or prophylaxis of a disorder mediated by the vasopressin receptor comprising administering an agonistic or antagonistic effective amount of a compound of the formula (II): 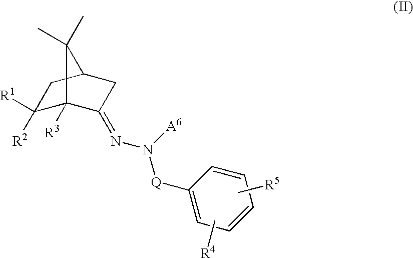
or its pharmaceutically acceptable salt thereof, wherein Q, R¹ and R² are defined above;
A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R⁴ and R⁵ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
optionally in a pharmaceutically acceptable carrier or diluent.

21. A method for the treatment or prophylaxis of a disorder mediated by the vasopressin receptor comprising administering an agonistic or antagonistic effective amount of a compound of the formula (III.1): 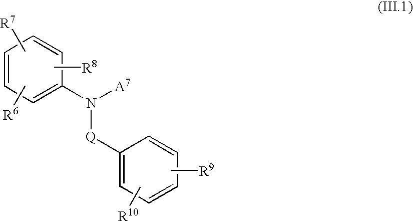
or its pharmaceutically acceptable salt thereof, wherein Q is defined above; and
A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
wherein if A⁷ and R^{6/8} independently come together to form a seven-membered bridged compound, then Q cannot be C(═O);
optionally in a pharmaceutically acceptable carrier or diluent.

22. A method for the treatment or prophylaxis of a disorder mediated by the vasopressin receptor comprising administering an agonistic or antagonistic effective amount of a compound of the formula (III.2): 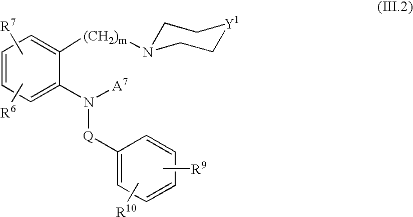
or its pharmaceutically acceptable salt thereof, wherein Q, A⁷, R⁶, R⁷, R⁹ and R¹⁰ are defined above;
m is 0 or 1;
Y¹ is O, S, NA⁸ or CR¹¹R¹²; and
A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹¹ and R¹² independently can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
optionally in a pharmaceutically acceptable carrier or diluent.

23. A method for the treatment or prophylaxis of a disorder mediated by the vasopressin receptor comprising administering an agonistic or antagonistic effective amount of a compound of the formula (III.3): 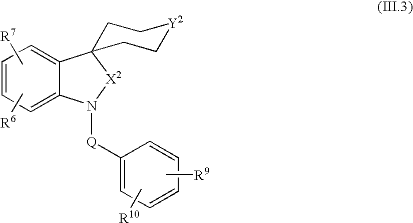
or its pharmaceutically acceptable salt thereof, wherein Q, R⁶, R⁷, R⁹ and R¹⁰ are defined above;
Y² is O, S, NA⁹ or CR¹⁵R¹⁶;
X² is C(═Z⁵) or CR¹⁷R¹⁸;
Z⁵ is O, S or NA¹⁰;
A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic or alkcarbonyl; and
R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
R¹⁵ or R¹⁶ independently cannot be the following moiety: 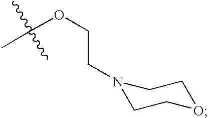
optionally in a pharmaceutically acceptable carrier or diluent.

24. A method for the treatment or prophylaxis of a disorder mediated by the vasopressin receptor comprising administering an agonistic or antagonistic effective amount of a compound of the formula (III.4): 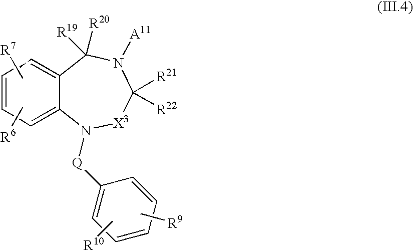
or its pharmaceutically acceptable salt thereof, wherein Q, R⁶, R⁷, R⁹ and R¹⁰ are defined above;
Y³ is O, S or NA¹¹;
X³ is C(═Z⁶);
Z⁶ is O, S or NA¹²;
A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
optionally in a pharmaceutically acceptable carrier or diluent.

25. A method for the treatment or prophylaxis of a disorder mediated by the vasopressin receptor comprising administering an agonistic or antagonistic effective amount of a compound of the formula (I): 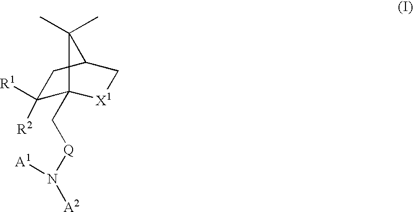
or its pharmaceutically acceptable salt thereof, wherein
X¹ is C(═Z¹) or CH₂;
Q is CH₂, C(═Z²), S, S(═Z³), (Z³═)S(═Z⁴), PA³, PA³(═O) or P(═O)₂;
Z¹ and Z² are independently O, S or NA⁴;
Z³ and Z⁴ are independently O or NA⁵ wherein Z³ and Z⁴ both cannot be NA⁵;
A¹, A², A³, A⁴ and A⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkcarbonyl wherein either Al or A² is an aromatic ring, preferably substituted with at least one carbonyl moiety; alternatively,
A¹ and A² individually can come together to form a bridged compound comprising of alkyl, alkenyl, alkynyl, cycloalkyl, cycloalkenyl, cycloalkynyl, aryl, alkaryl, arylalkyl, heterocyclic, heteroaromatic, alkcarbonyl, carbonyl, acyl, alkoxy, thiol, imine, sulfonyl, sulfanyl, sulfinyl, sulfamonyl, hydroxyl, ester, amide, phosphonyl, phosphinyl, phosphoryl, phosphine, imine, thioester, anhydride, oxime, hydrazine, carbamide, carbamate, thioether, residue of a natural or synthetic amino acid or a carbohydrate;
R¹ and R² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R¹ and R² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination or alternation with one or more other effective vasopressin receptor agonists or antagonists, optionally in a pharmaceutically acceptable carrier or diluent.

26. A method for the treatment or prophylaxis of a disorder mediated by the vasopressin receptor comprising administering an agonistic or antagonistic effective amount of a compound of the formula (II): 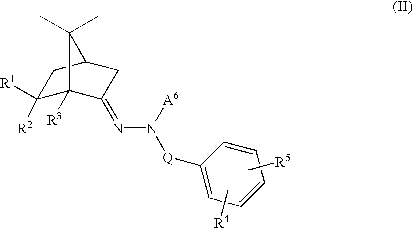
or its pharmaceutically acceptable salt thereof, wherein Q, R¹ and R² are defined above;
A⁶ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R³, R⁴ and R⁵ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrozinc, carbamate, thioether anhydride, residue of a natural or synthetic amino acid, or carbohydrate; alternatively
R⁴ and R⁵ as well as R^{4/5} and A⁶ independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination or alternation with one or more other effective vasopressin receptor agonists or antagonists, optionally in a pharmaceutically acceptable carrier or diluent.

27. A method for the treatment or prophylaxis of a disorder mediated by the vasopressin receptor comprising administering an agonistic or antagonistic effective amount of a compound of the formula (III.1): 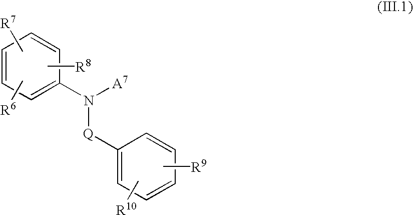
or its pharmaceutically acceptable salt thereof, wherein Q is defined above; and
A⁷ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R⁶, R⁷, R⁸, R⁹ and R¹⁰ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R⁶ and R⁷, R⁷ and R⁸, R⁹ and R¹⁰, A⁷ and R^{9/10}, and A⁷ and R^{6/8} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
wherein if A⁷ and R^{6/8} independently come together to form a seven-membered bridged compound, then Q cannot be C(═O);
in combination or alternation with one or more other effective vasopressin receptor agonists or antagonists, optionally in a pharmaceutically acceptable carrier or diluent.

28. A method for the treatment or prophylaxis of a disorder mediated by the vasopressin receptor comprising administering an agonistic or antagonistic effective amount of a compound of the formula (III.2): 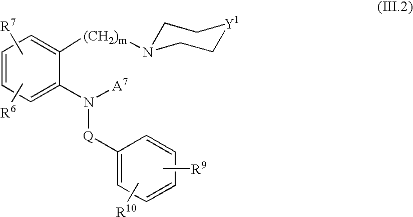
or its pharmaceutically acceptable salt thereof, wherein Q, A⁷, R⁶, R⁷, R⁹ and R¹⁰ are defined above;
m is 0 or 1;
Y¹ is O, S, NA⁸ or CR¹¹R¹²; and
A⁸ is hydrogen, alkyl, alkenyl, alkynyl, cycloalkyl, cycloalkenyl, cycloalkynyl, aryl, alkaryl, arylalkyl, heterocyclic, heteroaromatic or alkcarbonyl;
R¹¹ and R¹² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; alternatively
R¹¹ and R¹² independently can come together to form a spiro or bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination or alternation with one or more other effective vasopressin receptor agonists or antagonists, optionally in a pharmaceutically acceptable carrier or diluent.

29. A method for the treatment or prophylaxis of a disorder mediated by the vasopressin receptor comprising administering an agonistic or antagonistic effective amount of a compound of the formula (III.3): 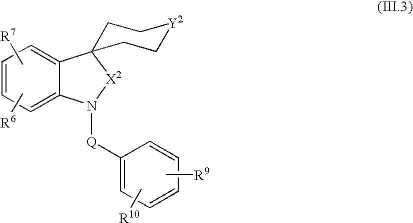
or its pharmaceutically acceptable salt thereof, wherein Q, R⁶, R⁷, R⁹ and R¹⁰ are defined above;
Y² is O, S, NA⁹ or CR¹⁵R¹⁶;
X² is C(═Z⁵) or CR¹⁷R¹⁸;
Z⁵ is O, S or NA¹⁰;
A⁹ and A¹⁰ are independently hydrogen, alkyl, alkenyl, alkynyl, cycloalkyl, cycloalkenyl, cycloalkynyl, aryl, alkaryl, arylalkyl, heterocyclic, heteroaromatic or alkcarbonyl; and
R¹⁵, R¹⁶, R¹⁷ and R¹⁸ are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁵ and R¹⁶ as well as R¹⁷ and R¹⁸ independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate; and
R¹⁵ or R¹⁶ independently cannot be the following moiety: 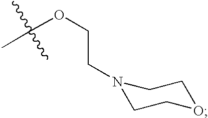
in combination or alternation with one or more other effective vasopressin receptor agonists or antagonists, optionally in a pharmaceutically acceptable carrier or diluent.

30. A method for the treatment or prophylaxis of a disorder mediated by the vasopressin receptor comprising administering an agonistic or antagonistic effective amount of a compound of the formula (III.4): 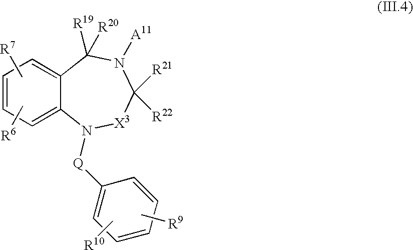
or its pharmaceutically acceptable salt thereof, wherein Q, R⁶, R⁷, R⁹ and R¹⁰ are defined above;
Y³ is O, S or NA¹¹;
X³ is C(═Z⁶);
Z⁶ is O, S or NA¹²;
A¹¹ and A¹² are independently hydrogen, alkyl, alkenyl, alkynyl, cycloalkyl, cycloalkenyl, cycloalkynyl, aryl, alkaryl, arylalkyl, heterocyclic, heteroaromatic, or alkcarbonyl;
R¹⁹, R²⁰, R²¹ and R²² are independently hydrogen, alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
R¹⁹ and R²⁰ as well as R²¹ and R²² independently can come together to form a spiro compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
A¹¹ and R^{19/20} or R^{21/22} independently can come together to form a bridged compound comprising alkyl, alkenyl, alkynyl, cycloalkyl, cycloalkenyl, cycloalkynyl, aryl, alkaryl, arylalkyl, heterocyclic, heteroaromatic, alkoxy, amino, halogen, silyl, thiol, sulfonyl, sulfanyl, sulfinyl, sulfamonyl, hydroxyl, ester, alkcarbonyl, carbonyl, acyl, thioester, acid halide, carboxylic acid, amide, imine, nitro, cyano, phosphonyl, phosphinyl, phosphoryl, phosphine, thioester, acid halide, anhydride, oxime, hydrazine, carbamate, thioether anhydride, residue of a natural or synthetic amino acid, or carbohydrate;
in combination or alternation with one or more other effective vasopressin receptor agonists or antagonists, optionally in a pharmaceutically acceptable carrier or diluent.

31. The method of any one of claims 19-30, wherein the disorder mediated by the vasopressin receptor is renal dysfunction.

32. The method of any one of claims 19-30, wherein the disorder mediated by the vasopressin receptor is hypertension.

33. The method of any one of claims 19-32, wherein the host is a human.
